(19) United States
(12) Patent Application Publication
(10) Pub. No.: US 2024/0199639 A1
(43) Pub. Date: Jun. 20, 2024

(54) PRETOMANID AMORPHOUS FORM

(71) Applicant: THE GLOBAL ALLIANCE FOR TB DRUG DEVELOPMENT, INC., New York, NY (US)

(72) Inventors: Rajneesh TANEJA, New York, NY (US); Poonam G. PANDE, Chapel Hill, NC (US)

(21) Appl. No.: 18/274,483

(22) PCT Filed: Feb. 1, 2022
(86) PCT No.: PCT/US2022/014750

§ 371 Date: Jul. 27, 2023

Related U.S. Application Data
(60) Provisional application No. 63/144,059, filed on Feb. 1, 2021.

(51) Int. Cl. C07D 498/04; A61K 9/00; A61K 9/10; A61K 9/48; A61K 31/5365

(52) U.S. Cl. CPC C07D 498/04; A61K 9/0053; A61K 9/10; A61K 9/4866; A61K 31/5365

(57) ABSTRACT

The present invention relates to pretomanid in amorphous form. The invention also relates to method of using the same, such as in a method of treating a mycobacterial infection.

### CROSS-REFERENCE TO RELATED APPLICATIONS

This application claims priority of U.S. Provisional Application No. 63/144,059, filed Feb. 1, 2021, the contents of which is hereby incorporated by reference in its entirety.

### FIELD OF THE INVENTION

The invention relates to an amorphous form of pretomanid. The amorphous form is useful in pharmaceutical preparations for the treatment of tuberculosis.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

Mycobacterium tuberculosis is the causative agent of tuberculosis (“TB”), a devastating infectious disease. It is estimated that about 2 million TB patients die each year globally. Failure to properly treat tuberculosis has caused global drug resistance in mycobacterium tuberculosis and thus rendering some medications ineffective.

Pretomanid (also known as “Pa” or “PA-824”) is a nitroimidazole anti-bacterial agent. As a TB therapy, it has many attractive characteristics—most notably its novel mechanism of action, its activity in vitro against all tested drug-resistant clinical isolates, and its activity as both a potent bactericidal and a sterilizing agent. In addition, the compound shows no evidence of mutagenicity in a standard battery of genotoxicity studies, no significant cytochrome P450 interactions, and no significant activity against a broad range of Gram-positive and Gram-negative bacteria. The IUPAC designation for pretomanid is (6S)-2-nitro-6-{[4-(trifluoromethoxy)benzyl]oxy}-6,7-dihydro-5H-imidazo[2,1-b][1,3]oxazine. Pretomanid is manufactured in crystalline form and has the following structure:

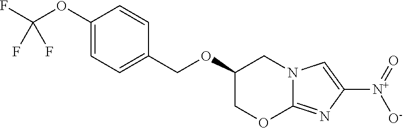 [SMILES: O=[N+]([O-])c1cn2c(n1)OC[C@@H](OCc1ccc(OC(F)(F)F)cc1)C2]

A need exists in the art, however, for a pretomanid form that exhibits better solubility.

### SUMMARY OF THE INVENTION

The present invention is directed to an amorphous form of pretomanid, pharmaceutical compositions thereof and methods of treatment.

### BRIEF DESCRIPTION OF THE DRAWINGS

The drawings described below are for illustrative purposes only and are not intended to limit the scope of the invention.

FIG. 1 depicts a PLM image of PA-824 raw material.

FIG. 2A depicts a XRPD pattern of PA-824 raw material. FIG. 2B depicts a TGA/DSC overlay of PA-824 raw material. FIG. 2C depicts a HPLC profile of PA-824 raw material.

FIG. 3 depicts a histogram of kinetic solubility results of ASDs in SGF and FaSSIF at 37° C.

FIG. 4A depicts a XRPD of Soluplus ASD prepared by Nanospray Drying. FIG. 4B depicts a XRPD of HPMC-ASLF ASD prepared by Nanospray Drying. FIG. 4C depicts a XRPD of Soluplus ASD after 1 week at ambient temperature. FIG. 4D depicts a DSC of Soluplus ASD after 1 week at ambient temperature. FIG. 4E depicts a XRPD of HPMC-ASLF ASD after 1 week at ambient temperature. FIG. 4F depicts a DSC of HPMC-ASLF ASD after 1 week at ambient temperature. FIG. 4G depicts profiles of kinetic solubility results of Soluplus and HPMC-ASLF ASDs in FaSSIF at 37° C.

FIG. 5A depicts a PLM of Soluplus ASD with 30% drug loading prepared by nanospray drying. FIG. 5B depicts a XRPD of Soluplus ASD with 30% drug loading prepared by nanospray drying. FIG. 5C depicts a PLM of Soluplus ASD with 40% drug loading prepared by nanospray drying. FIG. 5D depicts a XRPD of Soluplus ASD with 40% drug loading prepared by nanospray drying. FIG. 5E depicts a PLM of Soluplus ASD with 50% drug loading prepared by nanospray drying. FIG. 5F depicts a XRPD of Soluplus ASD with 50% drug loading prepared by nanospray drying.

FIG. 6A depicts a PLM of Soluplus ASD Scale up with 30% drug loading prepared by nanospray drying. FIG. 6B depicts a XRPD of Soluplus ASD Scale up with 30% drug loading prepared by nanospray drying.

FIG. 7 depicts profiles of kinetic solubility results of Soluplus ASDs in FaSSIF at 37° C.

FIG. 8A depicts the appearance of Soluplus ASD prepared by Hot Melt Extrusion. FIG. 8B depicts the appearance of HPMC-ASLF ASD prepared by Hot Melt Extrusion. FIG. 8C depicts kinetic solubility of HME ASDs without sieving. FIG. 8D depicts kinetic solubility of HME ASDs after sieving.

FIG. 9 depicts a HPLC profile of PA-824 ASD prepared by HME.

FIG. 10 depicts a PLM result of Soluplus ASD prepared by HME.

FIG. 11 depicts a PLM result of HPMCAS ASD prepared by HME.

FIG. 12 depicts a XRPD overlay of HME ASD with Soluplus and HPMC ASLF.

FIG. 13 depicts a mDSC result of Soluplus ASD prepared by HME.

FIG. 14 depicts a mDSC result of HPMC ASLF ASD prepared by HME.

FIG. 15 depicts a TGA result of Soluplus ASD prepared by HME.

FIG. 16 depicts a TGA result of HPMC ASLF ASD prepared by HME.

FIG. 17 depicts a XRPD result of nano suspension before Lyophilization.

FIG. 18 depicts a XRPD overlay of lyophilized nanosuspension powders after 10 days under various stress conditions.

FIG. 19 depicts a characterization of PA-824 API (A) XRD, (B) DSC Thermogram.

FIG. 20 depicts a wavelength scan for PA-824 (A) and a PA-824 Standard Curve (B).

FIG. 21 depicts a micro-evaporation Analysis of PA824 in Phosphate Buffer pH 6.8 Polymer control (no surfactant).

FIG. 22 depicts a micro-evaporation Analysis of PA824 in Phosphate Buffer pH 6.8 with Soluplus and Various Surfactants.

FIG. 23 depicts a micro-evaporation Analysis of PA824 in Phosphate Buffer pH 6.8 with Surfactant Controls (no Polymer).

FIG. 24 depicts a micro-evaporation Analysis of PA824 in Phosphate Buffer pH 6.8 with Two-Polymer Matrices.

FIG. 25 depicts a DSC Thermogram for HPMCAS-L. Kollidon VA64, and Soluplus: (A) DSC Thermogram for HPMCAS-L Alone, (B) DSC Thermogram for Kollidon VA64 Alone, (C) DSC Thermogram for Soluplus Alone.

FIG. 26 depicts a DSC Thermogram for SLS, TPGS, Poloxamer 407; (A) DSC Thermogram for SLS Alone; (B) DSC Thermogram for TPGS Alone; (C) DSC Thermogram for Poloxamer 407 Alone.

FIG. 27 depicts a DSC Thermogram for Soluplus+TPGS (60:10), Soluplus+Poloxamer 407 (60:10) and Soluplus+SLS (60:10): (A) DSC Thermogram for Soluplus+TPGS (60:10), (B) DSC Thermogram for Soluplus+Poloxamer 407 (60:10), (C) DSC Thermogram for Soluplus+SLS (60:10).

FIG. 28 depicts a DSC Thermograms for Physical Mixtures of API with Matrices 1, 2 and 3: (A) DSC Thermogram for API+Soluplus (30:70); (B) DSC Thermogram for API+HPMCAS-L (30:70); (C) DSC Thermogram for API+Kollidon VA64 (30:70).

FIG. 29 depicts a DSC Thermograms for Physical Mixtures of API with Matrices 4, 5 and 6: (A) DSC Thermogram for API+Soluplus+TPGS (30:60:10); (B) DSC Thermogram for API+Soluplus+Poloxamer 407 (30:60:10); (C) DSC Thermogram for API+Soluplus+SLS (30:60:10).

FIG. 30 depicts a XRD Overlay of SDD Trial 1 through 7.

FIG. 31 depicts a XRD and DSC for SDD1: (A) XRD and (B) DSC.

FIG. 32 depicts a XRD and DSC for SDD 2: (A) XRD and (B) DSC.

FIG. 33 depicts a XRD and DSC for SDD 3: (A) XRD and (B) DSC.

FIG. 34 depicts a XRD and DSC for SDD 2: (A) XRD and (B) DSC.

FIG. 35 depicts a XRD and DSC for SDD 3: (A) XRD and (B) DSC.

FIG. 36 depicts a mean (±SD) Plasma Concentrations of PA-824 Following Oral Administration of Four Different Capsule Formulations at 30 mg/monkey to Male Monkeys (n=4) in linear-linear scale (A) and in log-linear scale (B).

FIG. 37 depicts an individual Plasma Concentration Time Profiles of PA-824 Following Oral Administration of the Crushed Tablet in Capsule (C1) at 30 mg/monkey to Male Monkeys (A: linear-linear plot and B: log-linear plot).

FIG. 38 depicts an individual Plasma Concentration Time Profiles of PA-824 Following Oral Administration of the SDD1 in Capsule (C2) at 30 mg/monkey to Male Monkeys (A: linear-linear plot and B: log-linear plot).

FIG. 39 depicts an individual Plasma Concentration Time Profiles of PA-824 Following Oral Administration of the SDD2 in Capsule (C3) at 30 mg/monkey to Male Monkeys (A: linear-linear plot and B: log-linear plot).

FIG. 40 depicts an individual Plasma Concentration Time Profiles of PA-824 Following Oral Administration of SDD3 in Capsule.

FIG. 41 depicts induction time of PA-824 at 80 μg/mL in PBS pH 6.5 in presence of different grades of HPMCAS at 100 μg/mL.

FIG. 42 depicts PLM images of ASDs after incubation in HCl solution pH 1.6 for 1 hour (left) and shift to pH 6.5 for another 1 hour (right).

FIG. 43 depicts PXRD of ASDs before and after 1 hour incubation in HCl solution pH 1.6.

FIG. 44 depicts dissolution profiles of PTM ASDs at 10% drug loading with different HPMCAS grades in (A) PBS pH 6.5 and (B) pH shift from 1.6 to 6.5.

FIG. 45 depicts dissolution profiles of PTM ASDs at 10% drug loading with different HPMCAS grades in biorelevant media (A) FaSSIF V1 and (B) FaSSGF to FaSSIF V1.

FIG. 46 depicts dissolution profiles of PTM ASDs at (A) 10% drug loading and (B) 20% drug loading with different HPMCAS grades in FaSSIF V1.

FIG. 47 depicts dissolution profiles of PTM ASDs at (A) 10% drug loading and (B) 20% drug loading with different HPMCAS grades in FaSSGF to FaSSIF V1.

FIG. 48 depicts dissolution profiles of PTM ASDs at 10% drug loading in (A) PBS pH 6.5 and (B) pH shift from 1.6 to 6.5.

FIG. 49 depicts dissolution profiles of PTM ASDs at 10% drug loading in biorelevant media (A) FaSSIF V1 and (B) FaSSGF to FaSSIF V1.

FIG. 50 depicts dissolution profiles of PTM ASDs at drug loading of (A) 10%, (B) 15% and (C) 20% with HPMCAS-HF in FaSSIF V1.

FIG. 51 depicts ddissolution profiles of PTM ASDs at drug loading of (A) 10%, (B) 15% and (C) 20% with HPMCAS-HF in FaSSGF to FaSSIF V1.

FIG. 52 depicts dissolution profiles of PTM ASDs at 20% drug loading with HPMCAS-HF salts in biorelevant media (A) FaSSIF VI and (B) FaSSGF to FaSSIF V1.

FIG. 53 depicts dissolution profiles of PTM ASDs at (1) 20% drug loading, (2) 25% drug loading with promising HPMCAS-HF as salts and with TPGS in biorelevant media (A) FaSSIF V1 and (B) FaSSGF to FaSSIF V1.

FIG. 54 depicts a comparison of dissolution profiles of PTM ASDs at (A) 20% drug loading, (B) 25% drug loading with promising HPMCAS-HF salts in FaSSIF V1.

FIG. 55 depicts a comparison of dissolution profiles of PTM ASDs at 20% and 25% drug loading with promising HPMCAS-HF salts in FeSSIF V2 pH 5.8.

FIG. 56 depicts dissolution profiles of PA-824 200 mg reference tablets in biorelevant media.

FIG. 57 depicts dissolution profiles in FaSSIF of ASDs prepared by rotovap and spray drying in powder, capsule and tablet formulations.

FIG. 58 depicts dissolution profiles in FaSSIF of (A) ASDs prepared by rotovap and spray drying.

FIG. 59 depicts dissolution of PTM-HF-Tris ASD (rotovap) in capsule size 0 with/without lubricants.

FIG. 60 depicts dissolution of ASDs in capsules size 0 (A) with 2% Aerosil and (B) without Aerosil.

FIG. 61 depicts dissolution profiles in FaSSIF, FeSSIF and FaSSGF to FaSSIF of (A) reference PA-824 200 mg tablets, formulated spray dried ASD tablets at 20% drug loading with (B) HPMCAS-HF and (C) HPMCAS-HF-Tris salt.

FIG. 62 depicts dissolution of formulated spray dried ASD tablets with HPMCAS-HF and HPMCAS-HF-Tris salt in PBS pH 6.5 with surfactant, 0.5% CTAB.

FIG. 63 depicts dissolution of tablet of PA-824 spray dried ASDs in (A) FaSSIF VI and (B) FaSSGF to FaSSIF V1.

FIG. 64 depicts NMR data on stored spray dried ASD powders.

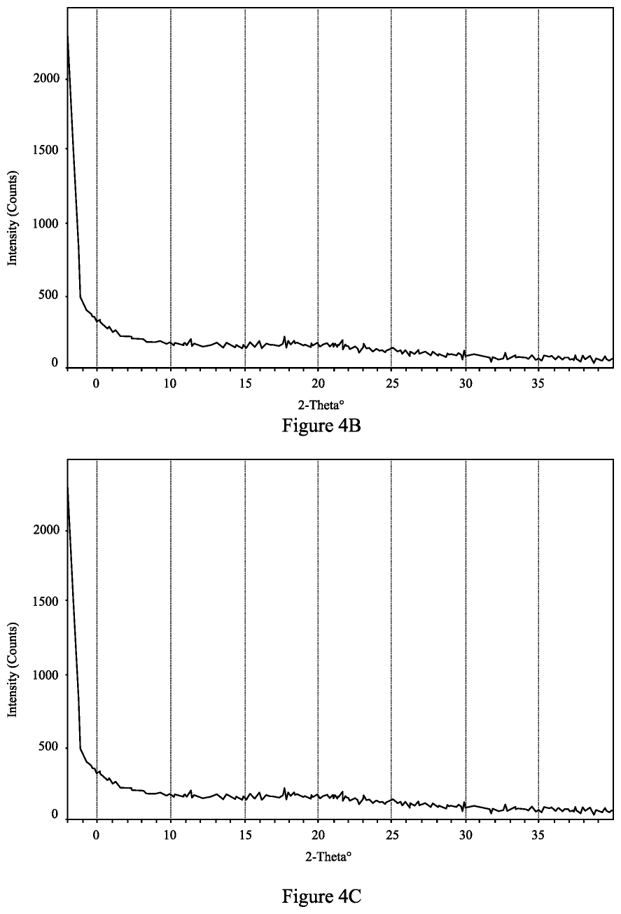

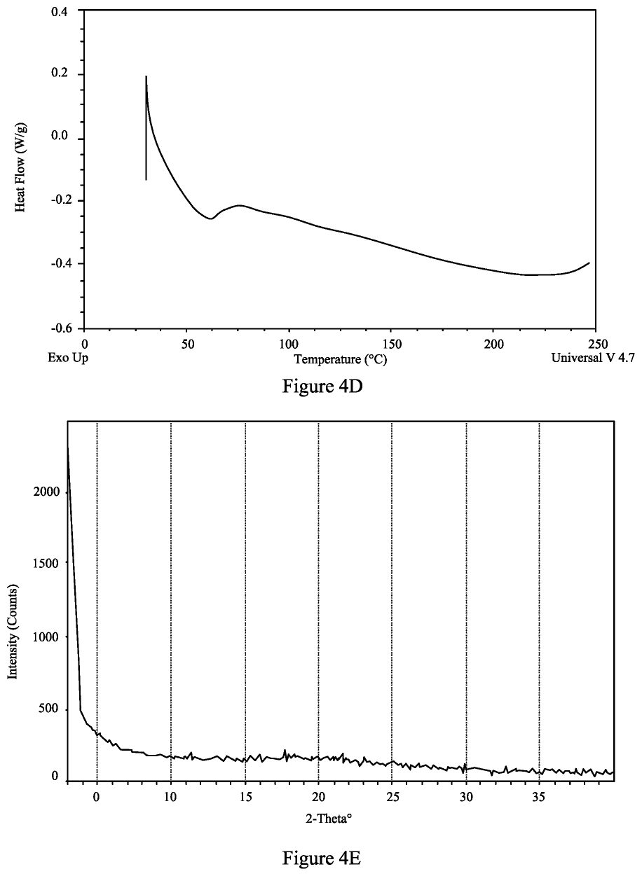

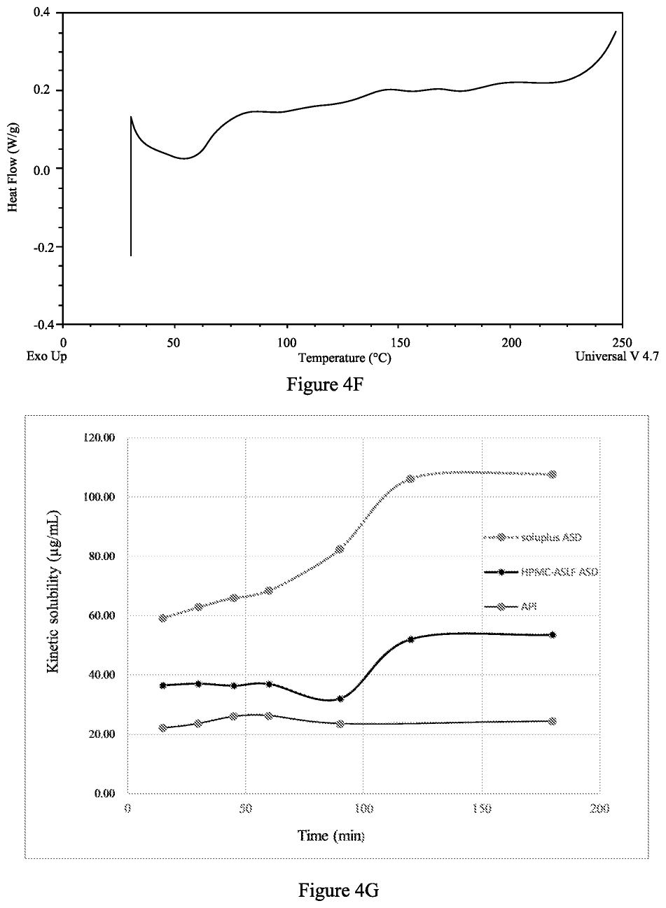

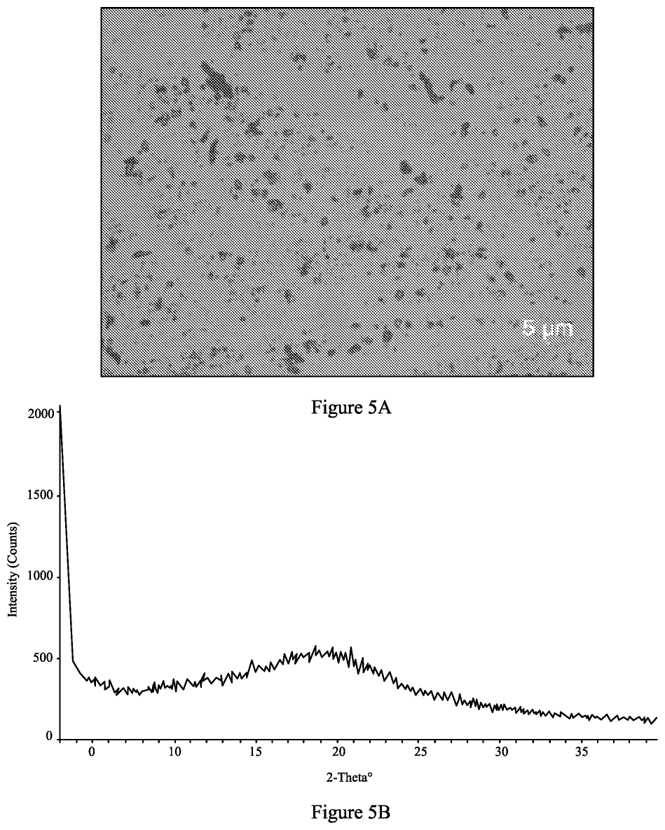

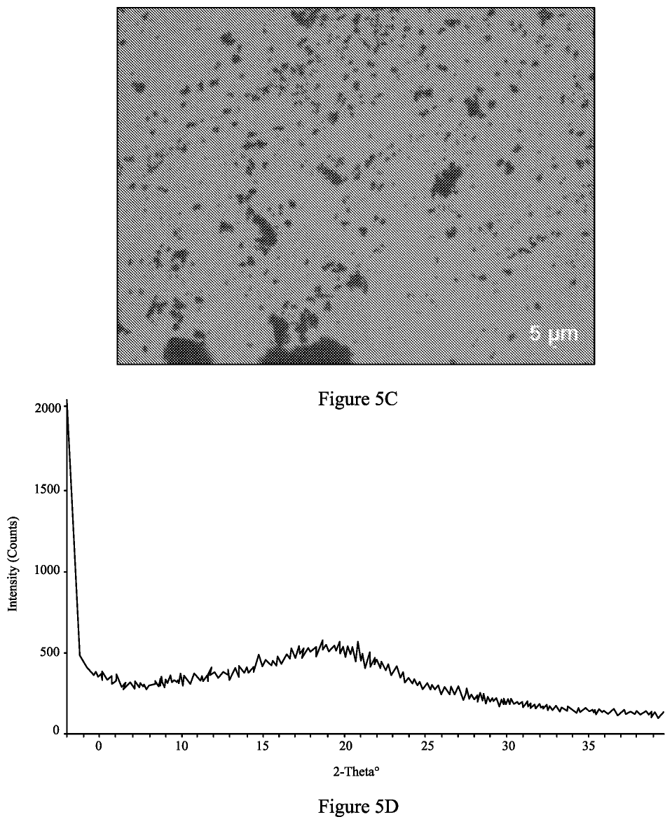

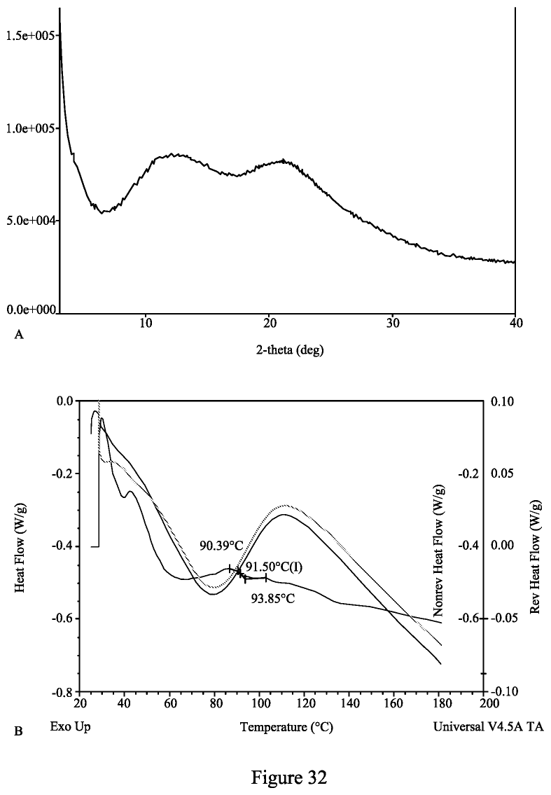

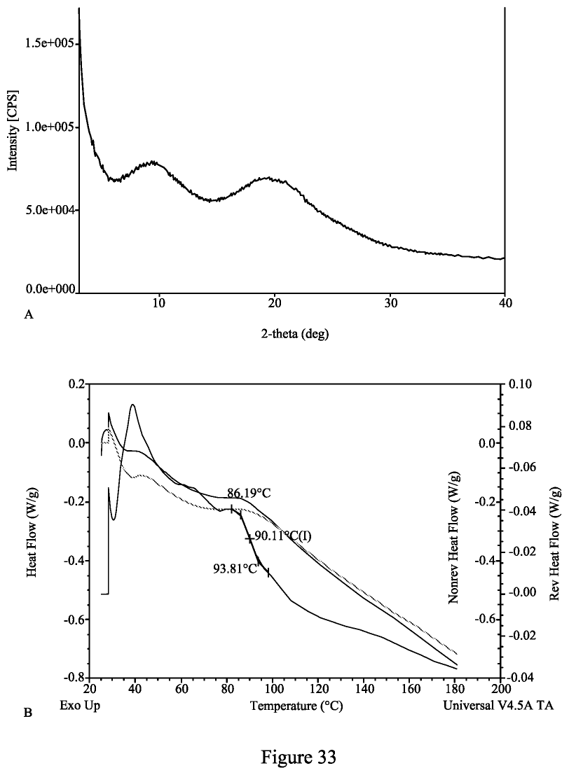

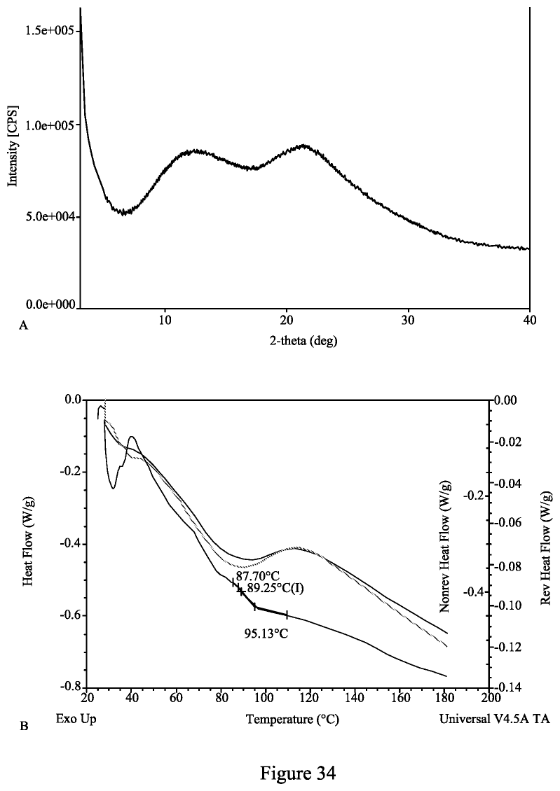

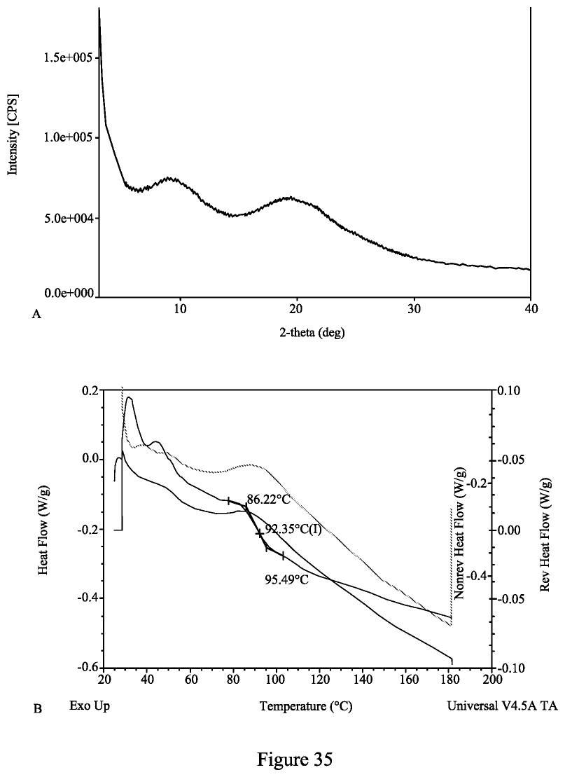

In FIGS. 4A, 4B, 4C, 4E, 5B, 5D, 5F, 6B, 12, 17, 18, 31A, 32A, 33A, 34A, and 35A, the vertical axis depicts the intensity, most often in counts, and the horizontal axis depicts 2-Theta(°).

### DETAILED DESCRIPTION OF THE INVENTION

It is to be understood that the descriptions of the present invention have been simplified to illustrate elements that are relevant for a clear understanding of the present invention, while eliminating, for the purpose of clarity, many other elements found in typical pharmaceutical compositions. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

### Certain Embodiments of the Invention

The present invention generally relates to pretomanid in amorphous form. Pretomanid (PA-824) raw material was obtained and characterized by PLM, XRPD, TGA/DSC, HPLC. Results showed that PA-824 was crystalline. DSC pattern showed single endothermic peak with an onset temperature of 149.77° C. (56.90 J/g). TGA results showed one stage of weight loss, which are 0.027% weight loss from 30° C. to 120° C. The purity of PA-824 raw material was 99.92%.

In the supersaturated solubility experiment, the equilibrium solubility of PA-824 in Fasted State Simulated Intestinal Fluid (FaSSIF) containing 2% DMSO was 51.40 μg/mL and the supersaturated solubility of PA-824 was 53.27 μg/mL.

Eight amorphous solid dispersions (“ASDs”) were made by solvent evaporation method and characterized by kinetic solubility. From the kinetic solubility data, it was noted that the solubility was improved for almost all ASDs at 1h in Simulated Gastric Fluid (SGF) at 1 h and in FaSSIF at 3 h compared to API. ASD with Soluplus showed the highest concentration in both SGF (41.60 μg/mL) and FaSSIF (52.05 μg/mL).

Soluplus and HPMC-ASLF were further selected for making ASDs by nano spray dry. Soluplus ASD and HPMC-ASLF ASD were checked by XRPD and HPLC. Soluplus ASD was amorphous and the purity was 99.82%. HPMC-ASLF ASD was also amorphous and the purity was 99.79%. From the data, it was noted that the solubility was improved for both ASDs in FaSSIF, compared to that of API in FaSSIF at 1 h (23.95 μg/mL) and 3 h (24.89 μg/mL). ASD with Soluplus showed higher concentration in FaSSIF.

Soluplus ASDs with different drug-loadings were prepared by nano spray drying and evaluated. Results of kinetic solubility suggested that Soluplus-ASDs with 30% API showed higher concentration in FaSSIF, compared to that of Soluplus-ASDs with 40% API or 50% API.

ASD with 30% drug-loading was scaled up successfully by nano spray drying. Soluplus ASD with 30% drug-loading was amorphous and the purity was 99.92%.

ASDs were prepared by HME by using Soluplus and HPMC-ASLF at ratio of 3:7 (w/w). Results of kinetic solubility showed that the solubility was improved for both ASDs in FaSSIF.

Nano suspension was prepared by Roller Mill and Planetary Ball Mill (PM400) in Vehicle 1 (2% PVP K12 and 0.05% Tween 80 in water (w/v)), Vehicle 2 (2% Poloxamer 188 and 0.05% Tween 80 in water (w/v)), Vehicle 3 (0.5% HPMC E5 and 0.05% Tween 80 in water (w/v)) and Vehicle 4 (2% Soluplus and 0.05% Tween 80 in water (w/v)).

Provided is an amorphous solid dispersion comprising pretomanid or a pharmaceutically acceptable salt thereof. This amorphous solid dispersion may comprise a pharmaceutically acceptable excipient or a polymer.

In embodiments, the pharmaceutically acceptable excipient or the polymer, is a polyvinyl caprolactam-polyvinyl acetate-polyethylene glycol graft copolymer, Hypromellose Acetate Succinate, Vinylpyrrolidone-vinyl acetate copolymer, copovidone, Polyvinylpyrrolidone or Povidone, poloxamer, or a basic methacrylate copolymer.

The amorphous form of the invention can be used in a pharmaceutical formulation for the treatment of TB. Pharmaceutical formulations according to the present invention comprise a combination according to the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared (Remington's Pharmaceutical Sciences (Mack Publishing Co., Easton, Pa.) Compositions intended for oral use may be prepared according to any method known to the art for the manufacture of pharmaceutical compositions and such compositions may contain one or more agents including antioxidants, sweetening agents, flavoring agents, coloring agents and preserving agents, in order to provide a palatable preparation. Tablets containing the active ingredient in admixture with non-toxic pharmaceutically acceptable excipient or auxiliary agents which are suitable for manufacture of tablets are acceptable. Suitable excipients or auxiliary agents include but are not limited to, for example, inert diluents, solubilizers, suspending agents, adjuvants, wetting agents, sweeteners, perfuming or flavoring substances, isotonic substances, colloidal dispersants and surfactants, including, but not limited to, charged phospholipids such as dimyristoylphosphatidylglycerin, algininic acid, alginates, acacia resin, gum arabic, 1,3-butylene glycol, benzalkonium chloride, colloidal silicon dioxide, cetosteryl alcohol, cetomacrogol emulsifying wax, casein, calcium stearate, cetylpyridine chloride, cetyl alcohol, cholesterol, calcium carbonate, CRODESTAS F-110, which is a mixture of sucrose stearate and sucrose distearate (Croda Inc.), clays, kaolin and bentonite, derivatives of cellulose and salts thereof, such as hydroxypropyl methylcellulose (HPMC), sodium carboxymethyl cellulose, carboxymethyl cellulose and salts thereof, methyl cellulose, hydroxyethyl cellulose, hydroxpropyl cellulose, hydroxypropyl methylcellulose phtalate, non-crystalline cellulose, dicalcium phosphate, dodecyltrimethylammonium bromide, dextrane, dialkylester of sodium sulfosuccinate (e.g. AEROSEL OT, American Cyanamid), gelatin, glycerol, glycerol monostearate, glucose, p-isononylphenoxypoly (glycidol), also known as Olin 10-G or 10-GR surfactant (Olin Chemicals, Stamford, Conn.); glucamides such as octanoyl-N-methylglucamide, decanoyl-N-methylglucamide and heptanoyl-N-methylglucamide, lactose, lecithin (phosphatides), maltosides such as n-dodecyl-beta-D-maltoside, mannitol, magnesium sterarate, magnesium aluminum silicates, oils such as cotton oil, seed oil, olive oil, castor oil and sesame oil; paraffin, potato starch, polyethylene glycol (e.g. CARBOWAX 3350, CARBOWAX 1450 and CARBOPOL 9340 (Union Carbide), polyoxyethylene alkyl ester (e.g. macrogolethers such as CETOMACROGOL 1000), polyoxyethylene sorbitol fatty acid esters (e.g. TWEENS, ICI Specialty Chemicals), polyoxyethylene castor oil derivatives, polyoxyethylene stearates, polyvinyl alcohol (PVA), polyvinylpyrrolidone (PVP), phosphates, 4-(1,1,3,3-tetramethylbutyl)phenol polymer with ethylene oxide and formaldehyde (also known as TYLOXAPOL, SUPERIONE and TRITON), poloxamers and polaxamines (e.g. PLURONICS F68LF, F87, F108 and TETRONIC 908, available from BASF Corporation, Mount Olive, N.J.), pyranosides such as n-hexyl-beta-D-glucopyranoside, n-decyl-beta-D-glucopyranoside, n-octyl-beta-D-glucopyranoside, quaternary ammonium compounds, silica, sodium citrate, starches, sorbitol esters, sodium carbonate, solid polyethylene glycols, sodium dodecyl sulfate, sodium lauryl sulfate (e.g. DUPONAL P, DuPont), stearic acid, sucrose, tapioca starch, talc, thioglucosides such as n-heptyl -.beta.-D-thioglucoside, tragacanth, triethanolamine, TRITON X-200 (Rohm and Haas); and the like.

Formulations for oral use may be also presented as hard gelatin capsules where the active ingredient is mixed with an inert solid diluent, for example pregelatinized starch, calcium phosphate or kaolin, or as soft gelatin capsules wherein the active ingredient is mixed with water or an oil medium, such as peanut oil, liquid paraffin or olive oil.

In one embodiment, provided is a method of treating a mycobacterial infection, comprising the step of administering a therapeutically effective amount of an amorphous form of pretomanid or an amorphous solid dispersion, to a patient in need thereof. In embodiments, the mycobacterial infection is caused by Mycobacterium tuberculosis, Mycobacterium avium, Mycobacterium kansasii, Mycobacterium abscessus or Mycobacterium chelonae. In embodiments, the patient is afflicted with tuberculosis (TB), multi-drug-resistant tuberculosis (MDR-TB), pre-extensively drug resistant (Pre-XDR-TB) or extensively drug-resistant tuberculosis (XDR-TB). In embodiments, the patient is thereby treated.

### Definitions

The articles “a” and “an” are used in this disclosure to refer to one or more than one (i.e., to at least one) of the grammatical object of the article.

A “subject” is a human, and the terms “subject” and “patient” are used interchangeably herein.

The term “treating,” with regard to a subject, encompasses, e.g., inducing inhibition, regression, or stasis of a disease or disorder; or curing, improving, or at least partially ameliorating the disorder; or alleviating, lessening, suppressing, inhibiting, reducing the severity of, eliminating or substantially eliminating, or ameliorating a symptom of the disease or disorder. “Inhibition” of disease progression or disease complication in a subject means preventing or reducing the disease progression and/or disease complication in the subject.

A “symptom” associated with a disease or disorder includes any clinical or laboratory manifestation associated with the disease or disorder and is not limited to what the subject can feel or observe.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration.

As used herein, a “unit dose”, “unit doses” and “unit dosage form(s)” mean a single drug administration entity/entities.

As used herein, “effective” or “therapeutically effective” when referring to an amount of a substance, for example an drug, refers to the quantity of the substance that is sufficient to yield a desired therapeutic response. In certain embodiments, an effective amount refers to an amount effective, at dosages and for periods of time necessary, to achieve the desired therapeutic or prophylactic result. A therapeutically effective amount of a compound or composition of the invention (e.g., an amorphous form) may vary according to factors such as the disease state, age, sex, and weight of the individual, and the ability of the compound or composition to elicit a desired response in the individual. A therapeutically effective amount encompasses an amount in which any toxic or detrimental effects of the compound or composition are outweighed by the therapeutically beneficial effects.

As used herein, a solid that is in the “amorphous” form means that it is in a non-crystalline state. Amorphous solids generally possess crystal-like short-range molecular arrangement, but no long-range order of molecular packing as are found in crystalline solids. The solid-state form of a solid, such as the drug substance in the amorphous dispersion, may be determined by Polarized Light Microscopy, X-Ray Powder Diffraction (XPRD), Differential Scanning calorimetry (DSC), or other standard techniques known to those of skill in the art.

The amorphous solid contains drug substance in a substantially amorphous solid-state form, e.g., at least about 50% of the drug substance in the dispersion is in an amorphous form, at least about 60% of the drug substance in the dispersion is in an amorphous form, at least about 70% of the drug substance in the dispersion is in an amorphous form, at least about 80% of the drug substance in the dispersion is in an amorphous form, at least about 90% of the drug substance in the dispersion is in an amorphous form, and at least about 95% of the drug substance in the dispersion is in amorphous form.

In some embodiments, at least about 90% (e.g., at least 95%, 96%, 97%, 98%, 99%, 99.5%, or even 99.9%, such as from 90% to 99.9%, from 90% to 99.5%, from 90% to 99%, from 90% to 98%, from 90% to 97%, from 90% to 96%, from 90% to 95%, from 95% to 99.9%, from 95% to 99.5%, from 95% to 99%, from 95% to 98%, from 95% to 97%, and from 95% to 96%) of the pretomanid is in amorphous form.

Poloxamer 407 is a Polyethylene-Polypropylene Glycol. Poloxamer 407 is a hydrophilic non-ionic surfactant of a general class of copolymers known as poloxamers. A poloxamer is a synthetic block copolymer of ethylene oxide and propylene oxide. Poloxamer 407 is a triblock copolymer consisting of a central hydrophobic block of polypropylene glycol flanked by two hydrophilic blocks of polyethylene glycol (PEG). The approximate length of the two PEG blocks is most typically 101 repeat units, while the approximate length of the propylene glycol block is most typically 56 repeat units.

Eudragit® E PO (EUD EPO) is basic methacrylate copolymer manufactured by Evonik Röhm GmbH. This amino methacrylate copolymer is a polymerized copolymer of dimethylaminoethyl methacrylate, butyl methacrylate, and methyl methacrylate with a ratio of 2:1:1.

Soluplus® is a polyvinyl caprolactam-polyvinyl acetate-polyethylene glycol graft copolymer.

HPMCAS LF or HPMC ASLF are Hypromellose Acetate Succinates. Hypromellose Acetate Succinate is a mixture of acetic acid and monosuccinic acid esters of hydroxypropyl methylcellulose. Herein, “hydroxypropyl methylcellulose acetate succinate” polymer is also referred to as HPMCAS, and is commonly known in the field of polymers as CAS registry number 71138-97-1. HPMCAS has many chemical common synonyms, such as: Hypromellose Acetate Succinate; HPMC-AS; Cellulose, 2-hydroxypropylmethylether, acetate, hydrogen butanedioate. Examples of the product include HPMCAS also known as Shin-Etsu AQOAT. The polymer is available in micronized grade (LF, MF, HF) with mean particle size of 5 microns (rim) or granular grade (LG, MG, HG) with mean particle size of 1 mm. In certain embodiments the polymer is in the form of finely divided solid particles having an average diameter ranging from about 0.1 to about 10 microns. This example of HPMCAS is a product defined as containing not less than 4% and not more than 18% of succinoyl groups, which are only free carboxylic groups in the compound and not less than 5% and not more than 14% acetyl groups present in the compound. The degree of succinoyl and acetyl substitutions defines the grade (L, M or H), the higher the acetyl content, the lower the succinoyl content. For example, HPMCAS may include the following components:

| | Acetyl | Succinoyl | Methoxyl | Hydroxypropoxy |
|---|---|---|---|---|
| HPMCAS | 5-14% | 4-18% | | |
| HPMCAS LF | 5-9% | 14-18% | 20-24% | 5-9% |
| HPMCAS MF | 7-11% | 10-14% | 21-25% | 5-9% |
| HPMCAS HF | 10-14% | 4-8% | 22-26% | 6-10% |

Kollidon VA64 is a vinylpyrrolidone-vinyl acetate copolymer or Copovidone. Copovidone is a copolymer of 1-vinyl-2-pyrrolidone and vinyl acetate in the mass proportion of 3:2. Kollidon K30 is a Polyvinylpyrrolidone or a Povidone. Povidone is also classified as a synthetic polymer consisting essentially of linear 1-vinyl-2-pyrrolidinone groups.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose, sucralose or saccharin.

Oil suspensions may be formulated by suspending the active ingredient in a vegetable oil, such as arachis oil, olive oil, sesame oil or coconut oil, or in a mineral oil such as liquid paraffin The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid, BHT, etc.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

According to another embodiment of the invention, provided is a pharmaceutical composition in the form of a dispersible tablet. Dispersible tablets are intended to be dispersed in water before administration, providing a homogeneous dispersion. Dispersible tablets disintegrate within, for example, 3 minutes using water at 15-25° C.

The pharmaceutical compositions of the invention may also be in the form of oil-in-water emulsions or liposome formulations. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. As noted above, formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion.

Compositions of the present invention are administered to a human or other mammal in a safe and therapeutically effective amount as described herein. These safe and therapeutically effective amounts will vary according to the type and size of mammal being treated and the desired results of the treatment. A “therapeutically effective amount” is, e.g., an amount effective for treating tuberculosis. The term “treating”, with regard to a subject, refers to improving at least one symptom of the subject's disorder. Treating can be curing, improving, or at least partially ameliorating the disorder.

Any of the various methods known by persons skilled in the art for packaging tablets, caplets, or other solid dosage forms suitable for oral administration, that will not degrade the components of the present invention, are suitable for use in packaging. The combinations may be packaged in glass and plastic bottles. Tablets, caplets, or other solid dosage forms suitable for oral administration may be packaged and contained in various packaging materials optionally including a desiccant e.g. silica gel. Packaging may be in the form of unit dose blister packaging. For example, a package may contain one blister tray of tenofovir DF and another blister tray of emtricitabine pills, tablets, caplets, or capsule. A patient would take one dose, e.g. a pill, from one tray and one from the other. Alternatively, the package may contain a blister tray of the co-formulated combination of tenofovir DF and emtricitabine in a single pill, tablet, caplet or capsule. As in other combinations and packaging thereof, the combinations of the invention include physiological functional derivatives of tenofovir DF and FTC.

The packaging material may also have labeling and information related to the pharmaceutical composition printed thereon. Additionally, an article of manufacture may contain a brochure, report, notice, pamphlet, or leaflet containing product information. This form of pharmaceutical information is referred to in the pharmaceutical industry as a “package insert.” A package insert may be attached to or included with a pharmaceutical article of manufacture. The package insert and any article of manufacture labeling provides information relating to the pharmaceutical composition. The information and labeling provide various forms of information utilized by health-care professionals and patients, describing the composition, its dosage and various other parameters required by regulatory agencies such as the United States Food and Drug Agency.

### EXAMPLES

The following examples further describe and demonstrate particular embodiments within the scope of the present invention. Techniques and formulations generally are found in Remington's Pharmaceutical Sciences (Mack Publishing Co., Easton, Pa.). The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Raw Material Characterization

Starting material: PA-824 (Pretomanid) as a white powder. Other reagents and excipients are as follows: (Name, Grade, Manufacturer) Water, Purified, WuXi; DMSO, HPLC, SIGMA, SGF (pH 1.8), N/A, WuXi; FaSSIF/FeSSIF/FaSSGF biorelevant powder, N/A, Biorelevant; FaSSIF (pH 6.51), N/A, WuXi; Acetonitrile, HPLC, Merck; TFA, HPLC, Merck; Kollidon 30 (PVP K30), N/A, BASF; Kollidon VA64 (PVP VA64), N/A, BASF; Soluplus, N/A, BASF; Methocel E5 Premium LV Hydroxypropyl Methylcellulose (HPMC E5), N/A, BASF; HPMCAS (HPMC-ASLF), N/A, Shin-Etsu or HPMCAS (LF), N/A, Ashland; Eudragit E PO, N/A, Evonik; Eudragit L100, N/A, Evonik; Polyethylene glycol 8000, N/A, The Dow Chemical Company, Kollidon 12 (PVP K12), N/A, BASF; Tween 80, N/A, Fluka; Poloxamer 188, N/A, BASF.

### Testing Media Preparation

Standard buffer solutions may be prepared by appropriate combinations. The details are shown in the Table 1.

TABLE 1
Details of buffer solutions preparation

| Buffer | Details |
|---|---|
| SGF | Transfer about 950 mL of deionized water to a 1 L volumetric flask. Add 1.4 mL |
| (pH 1.8) | concentrated HCl (12N) and 2 g of NaCl and stir to ensure homogeneity. Dilute to 1 L |
| | with deionized water. Check pH, it should be 1.8. |
| FaSSIF | Weigh 0.042 g of Sodium hydroxide pellets, 0.3438 g of Monobasic sodium phosphate |
| (pH 6.5) | (Anhydrous), 0.6186 g of Sodium chloride in about 0.09 L of purified water. Adjust the |
| | pH to 6.5 with Sodium hydroxide 1N or Hydrochloric acid 1N. Make up to volume (0.1 |
| | L) with purified water at room temperature. |
| | Add 0.224 g of FaSSIF/FeSSIF/FaSSGF biorelevant powder to about 0.05 L of buffer. |
| | Stir until powder is completely dissolved. Make up to volume (0.1 L) with buffer at |
| | room temperature. |

### Physical and Chemical Characterization

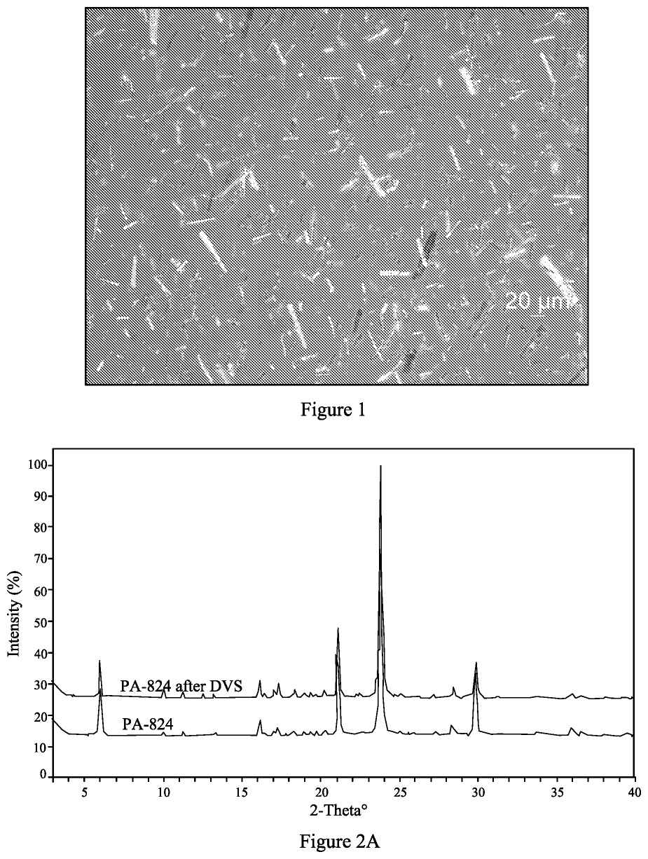

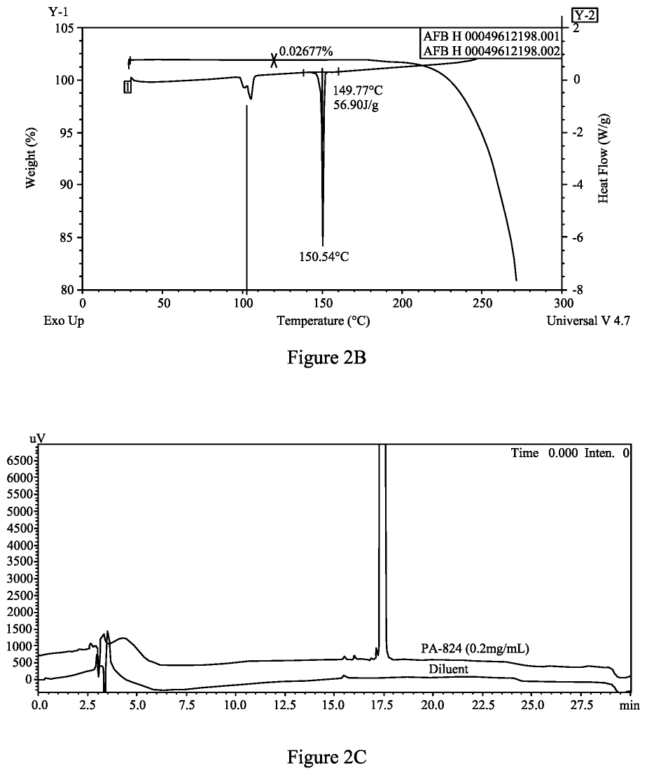

The raw material of the compound was characterized by PLM, XRPD, TGA/DSC. Results are provided in FIGS. 1, 2A and 2B, respectively. PA-824 is a white powder, and it displays birefringence under polarized light microscope (FIG. 1) and is crystalline by XRPD (FIG. 2A). DSC pattern (FIG. 2B) showed endothermic peak with an onset temperature of 149.77° C. (56.90 J/g). TGA results (FIG. 2B) showed one stage of weight loss of 0.027% weight loss from 30° C. to 120° C.

### Purity Test

Appropriately 2 mg of the compound was accurately weighed into a glass vial, then added diluent (AsCN/water, 50/50) and sonicated for 2 minutes to dilute the target concentration of 0.2 mg/mL. The solution was equilibrated to room temperature and then analyzed by HPLC. Based on the result, the purity of PA-824 raw material as received was 99.92%. The typical HPLC profile is shown in FIG. 2C.

### Supersaturated Solubility Measurement

Approximately 5 mg of PA-824 was weighed out into a glass vial, followed by the addition of a certain volume of FaSSIF containing 2% DMSO to get a target concentration of 5 mg/mL, then stirred at 700 rpm, 25° C. for 18 hrs, then centrifuged at 14,000 rpm for 10 min, the supernatant was analyzed by HPLC. The equilibrium solubility of PA-824 in FaSSIF containing 2% DMSO was 51.40 μg/mL.

Approximately 100 mg of PA-824 was weighed out into a glass vial, followed by the addition of DMSO to get a target concentration of 50 mg/mL, sonicated to get clear solution, then diluted the solution with DMSO at 8 different concentrations as stock solutions. Then 20 μL of each DMSO stock solution was added to FaSSIF at 1:49 to make the final DMSO concentration at 2% v/v. The samples were stirred at room temperature for 16 min, centrifuged and supernatants analyzed by HPLC. Table 2 shows the supersaturated solubility of PA-824 is 53.27 μg/mL.

TABLE 2
Supersaturated solubility results
of PA-824 in FaSSIF with 2% DMSO

| No. | Target conc.(μg/mL) | Measured conc.(μg/mL) |
|---|---|---|
| 1 | 51.40 | 53.08 |
| 2 | 102.80 | 53.27 |
| 3 | 205.60 | 30.82 |
| 4 | 411.20 | 32.98 |
| 5 | 514.00 | 29.48 |
| 6 | 616.80 | 28.71 |
| 7 | 771.00 | 30.31 |
| 8 | 1028.00 | 30.32 |

### Example 1, Part A

### Preparation and Evaluation of Amorphous Solid Dispersions by Vacuum Drying

### Preparation of ASDs by Vacuum Drying

Eight polymers, namely, PVP K30, PVP VA64, Soluplus, HPMCE5, HPMC-ASLF, EUD EPO, EUD L100 and PEG8000 were selected for making ASDs. API to polymer ratios with the ratio of 1:4 (w/w) was used for ASD preparation. To make ASDs, about 20 mg of the compound and 80 mg of corresponding polymers were weighed into 40 ml vials, 2 mL of solvent (DCM/MeOH=1/1 v/v) was added and dissolved, the solution was evaporated under vacuum at 35° C. overnight, and the resultant solids were ASDs.

Eudragit® E PO (EUD EPO) is a basic methacrylate copolymer manufactured by Evonik Röhm GmbH. This amino methacrylate copolymer is a polymerized copolymer of dimethylaminoethyl methacrylate, butyl methacrylate, and methyl methacrylate with a ratio of 2:1:1.

### Kinetic Solubility Test of ASDs in Biorelevant Media

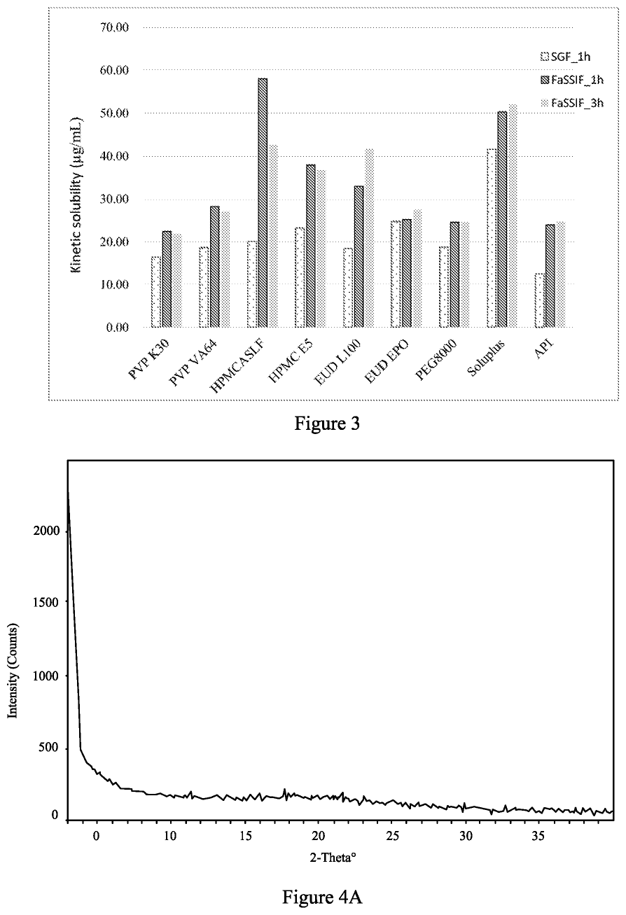

10 mL of SGF was added into 8 vials containing each ASD prepared by vacuum drying as described above, then the suspensions were shaken using a thermomixer at 37° C. under 100 rpm, 300 μL of suspensions were withdrawn at 1 h and the pH values were recorded. After that, 10 mL of FaSSIF was added into the suspensions containing SGF, 300 μL of suspensions were withdrawn at 1 h and 3 h, the pH values in 3 h were also recorded. The samples of suspensions were centrifuged in 96-well plates at 3000 rpm for 2 min. The supernatants were diluted with the diluent (ACN/water, 50/50) and analyzed by HPLC. Table 3 shows the kinetic solubility results of eight ASDs in SGF and FaSSIF. FIG. 3 shows the histograms of kinetic solubility data. From the data, it was noted that the solubility was improved for almost all ASDs at 1 h in SGF at 1 h and in FaSSIF at 3 h compared to API. ASD with Soluplus showed the highest concentration in both SGF (41.60 μg/mL) and FaSSIF (52.05 μg/mL).

TABLE 3
Kinetic solubility results of ASDs in SGF and FaSSIFat 37° C.

| | | | SGF | | FaSSIF | | |
|---|---|---|---|---|---|---|---|
| | | | Conc. | | Conc. | Conc. | |
| | | | (μg/ | pH | (μg/ | (μg/ | pH |
| | | Appear- | mL) | at | mL) | mL) | at |
| No. | ASD formulation | ance | (1 hr) | 1 h | (1 hr) | (3 hr) | 3 h |
| 1 | API + PVP K30 | white | 16.48 | 2.28 | 22.46 | 21.87 | 6.39 |
| | | powder | | | | | |
| 2 | API + PVP VA64 | white | 18.72 | 2.27 | 28.25 | 27.17 | 6.15 |
| | | powder | | | | | |
| 3 | API + EUD EPO | white | 24.86 | 2.51 | 25.28 | 27.60 | 6.47 |
| | | powder | | | | | |
| 4 | API + EUD L100 | Gel film | 18.53 | 2.27 | 33.01 | 41.76 | 5.85 |
| 5 | API + HPMC E5 | white | 23.26 | 2.26 | 37.94 | 36.76 | 6.14 |
| | | powder | | | | | |
| 6 | API + HPMC ASLF | Gel film | 20.17 | 2.28 | 58.05 | 42.56 | 5.93 |
| 7 | API + PEG 8000 | white | 18.62 | 2.27 | 24.62 | 24.59 | 6.18 |
| | | powder | | | | | |
| 8 | API + Soluplus | white | 41.60 | 2.27 | 50.28 | 52.05 | 6.18 |
| | | powder | | | | | |
| 9 | API | white | 12.46 | 2.27 | 23.95 | 24.89 | 6.18 |
| | | powder | | | | | |

### Example 1, Part B

### Preparation and Evaluation of Amorphous Solid Dispersions by Nanospray Drying

Soluplus and HPMC-ASLF were further selected for making ASDs. API to polymer ratio of 1:4 (w/w) was used for ASD preparation. To make ASDs, about 200 mg of the compound and 800 mg of corresponding polymers were weighed and dissolved in acetone (for Soluplus) or methanol (for HPMC-ASLF) with the concentration of total solid at 10 mg/mL and 5 mg/mL, respectively. After that, the solvents were removed by nanospray drying. The products were collected and stored in the vacuum drying oven at 35° C. overnight. The ASDs were characterized by XRPD, DSC and HPLC (Tables 4 and 5).

TABLE 4
Details of ASDs prepared by nanospray drying

| | | | Drug- | | |
|---|---|---|---|---|---|
| Sample | | Yield | loading | Purity | |
| Name | Appearance | (%) | (%) | (%) | XRPD |
| Soluplus | White | 48.92 | 20.79 | 99.82 | FIG. 4A |
| ASD | powder | | | | |
| HPMC- | White | 70.94 | 22.28 | 99.79 | FIG. 4B |
| ASLF ASD | powder | | | | |

TABLE 5
Details of ASDs prepared by nanospray
drying at ambient temperature at 1 week

| Sample | | Purity | | |
|---|---|---|---|---|
| Name | Appearance | (%) | XRPD | DSC |
| Soluplus | White | 99.71 | FIG. 4C | FIG. 4D |
| ASD | powder | | | |
| HPMC- | White | 99.63 | FIG. 4E | FIG. 4F |
| ASLF | powder | | | |
| ASD | | | | |

### Kinetic Solubility Test of ASDs in Biorelevant Media

10 mL of FASSIF was added into 8 vials containing each ASD prepared before by nanospray drying, respectively, then the suspensions were shaken using a thermomixer at 37° C. under 100 rpm, 300 μL of suspensions were withdrawn at 15, 30, 45, 60, 90, 120 and 180 mins. The samples of suspensions were centrifuged in 96-well plates at 3000 rpm for 5 min. The supernatants were diluted with the diluent (ACN/water, 50/50) and analyzed by HPLC. Table 6 is the kinetic solubility results of ASDs in FaSSIF.

FIG. 4G shows the profiles of kinetic solubility data. From the data, it was noted that the solubility was improved for both ASDs in FaSSIF, compared to that of API in FaSSIF. ASD with Soluplus showed higher concentration in FaSSIF.

TABLE 6
Kinetic solubility results of ASDs in FaSSIF at 37° C.

| | Solubility (μg/mL) | | | | | | |
|---|---|---|---|---|---|---|---|
| | 15 | 30 | 45 | 60 | 90 | 120 | 180 |
| Samples | min | min | min | min | min | min | min |
| API | 22.15 | 23.66 | 26.05 | 26.33 | 23.64 | — | 24.44 |
| ASD: 20% | 59.21 | 62.79 | 65.91 | 68.46 | 82.46 | 106.15 | 107.69 |
| API + 80% | | | | | | | |
| Soluplus | | | | | | | |
| ASD: 20% | 36.53 | 37.06 | 36.37 | 36.93 | 32.00 | 52.05 | 53.55 |
| API + 80% | | | | | | | |
| HPMC-ASLF | | | | | | | |

### Example 1, Part C

### Preparation and Evaluation of Soluplus ASDs with Different Drug-Loadings by Nanospray Drying

API to Soluplus ratios given in Table 7 were used for ASD preparation. To make ASDs, a certain amount of the compound and Soluplus were weighed and dissolved in acetone with the concentration of total solid at 10 mg/mL. After that, the solvents were removed by nanospray drying using parameters provided in Table 4. The products were collected and stored in the vacuum drying oven at 30° C. overnight. The ASDs were characterized by PLM, XRPD and HPLC.

TABLE 7
Details of Soluplus-ASDs with different
drug loading prepared by nanospray drying

| | API to | | | Drug- | | | |
|---|---|---|---|---|---|---|---|
| | Soluplus | Appear- | Yield | loading | Purity | | |
| No. | ratio | ance | (%) | (%) | (%) | PLM | XRPD |
| 1 | 30% API + | White | 52.05 | 30.24 | 99.9 | FIG. | FIG. |
| | 70% | powder | | | | 5A | 5B |
| | Soluplus | | | | | | |
| 2 | 40% API + | White | 59.73 | 40.05 | 99.9 | FIG. | FIG. |
| | 60% | powder | | | | 5C | 5D |
| | Soluplus | | | | | | |
| 3 | 50% API + | White | 17.26 | 44.07 | 99.9 | FIG. | FIG. |
| | 50% | powder | | | | 5E | 5F |
| | Soluplus | | | | | | |

| | | | |
|---|---|---|---|
| STD | N/A | 99.9 | N/A |

### Kinetic Solubility Test of Soluplus ASDs in Biorelevant Media

10 mL of FaSSIF was added into 3 vials containing each ASD (equivalent to 5 mg API) prepared by nanospray drying, respectively, then the suspensions were shaken using a thermomixer at 37° C. under 100 rpm, 300 μL of suspensions were withdrawn at 15, 30, 45, 60, 90, 120 and 180 mins. The samples of suspensions were centrifuged at 3000 rpm for 5 min. The supernatants were diluted with the diluent (ACN/water, 75/25) and analyzed by HPLC. Table 8 provides the kinetic solubility results of ASDs in FaSSIF.

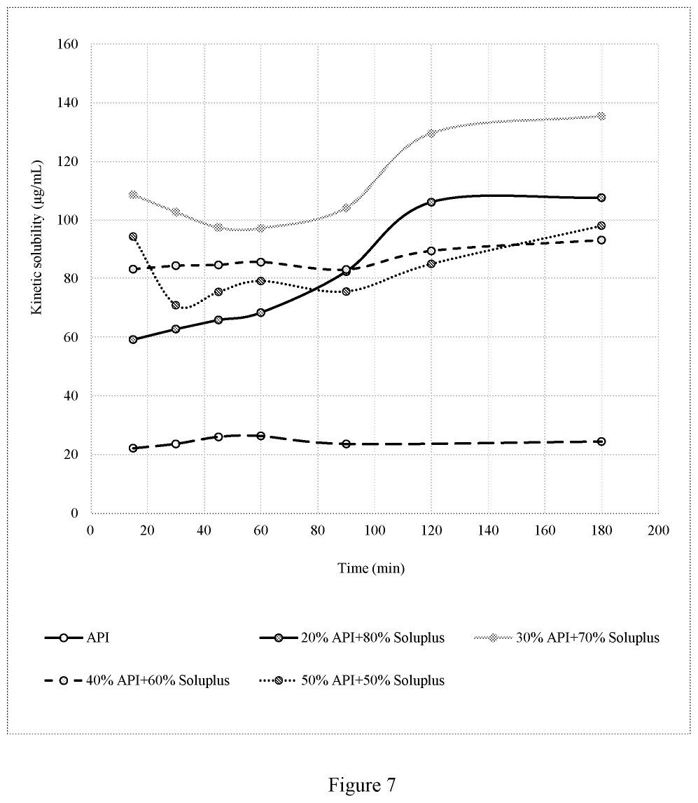

FIG. 7 shows the profiles of kinetic solubility data. From the data, it was noted that ASD with 30% API and 70% Soluplus showed highest concentration in FaSSIF.

TABLE 8
Kinetic solubility results of Soluplus ASDs in FaSSIF at 37° C.

| | Solubility (μg/mL) | | | | | | |
|---|---|---|---|---|---|---|---|
| | 15 | 30 | 45 | 60 | 90 | 120 | 180 |
| ASD | min | min | min | min | min | min | min |
| API | 22.15 | 23.66 | 26.05 | 26.33 | 23.64 | — | 24.44 |
| 20% | 59.21 | 62.79 | 65.91 | 68.46 | 82.46 | 106.15 | 107.69 |
| API + | | | | | | | |
| 80% | | | | | | | |
| Soluplus | | | | | | | |
| 30% | 108.64 | 102.77 | 97.45 | 97.28 | 104.19 | 129.68 | 135.52 |
| API + | | | | | | | |
| 70% | | | | | | | |
| Soluplus | | | | | | | |
| 40% | 83.27 | 84.43 | 84.73 | 85.72 | 83.13 | 89.51 | 93.18 |
| API + | | | | | | | |
| 60% | | | | | | | |
| Soluplus | | | | | | | |
| 50% | 94.40 | 70.94 | 75.51 | 79.22 | 75.66 | 85.04 | 98.05 |
| API + | | | | | | | |
| 50% | | | | | | | |
| Soluplus | | | | | | | |

### Example 1, Part D

### ASD Scale Up By Nanospray Drying

ASD with 30% API and 70% Soluplus was further selected for scale up. To make ASDs, 3 g of compound and 7 g of Soluplus were weighed and dissolved in acetone with the concentration of total solid at 10 mg/mL. After that, the solvents were removed by nanospray drying. The products were collected and stored in the vacuum drying oven at 30° C. overnight. The ASDs were characterized by PLM, XRPD and HPLC.

From the data (Table 9), characterizations of ASD with 30% drug loading which was scaled up were similar to the ASD with 30% drug loading prepared before.

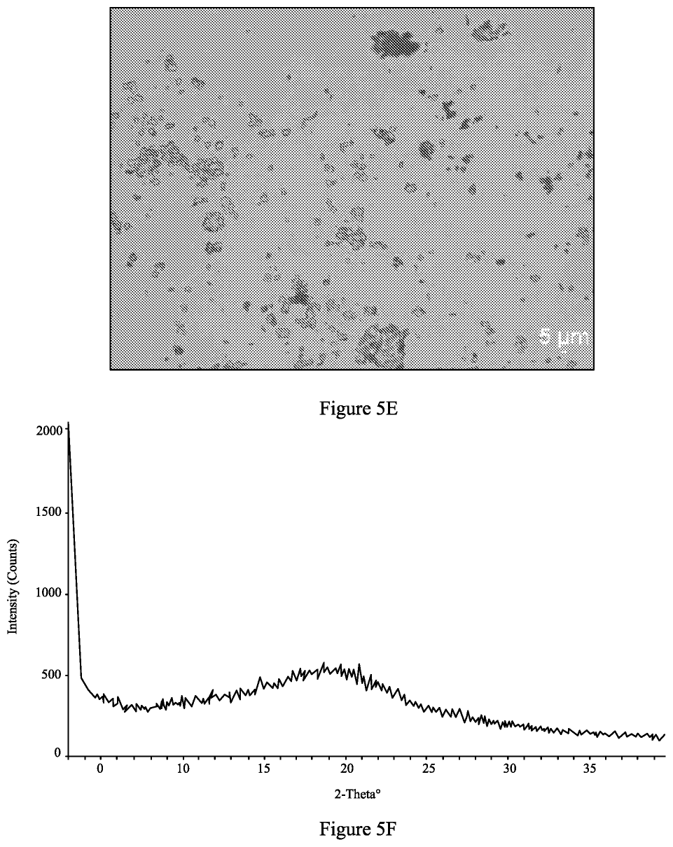

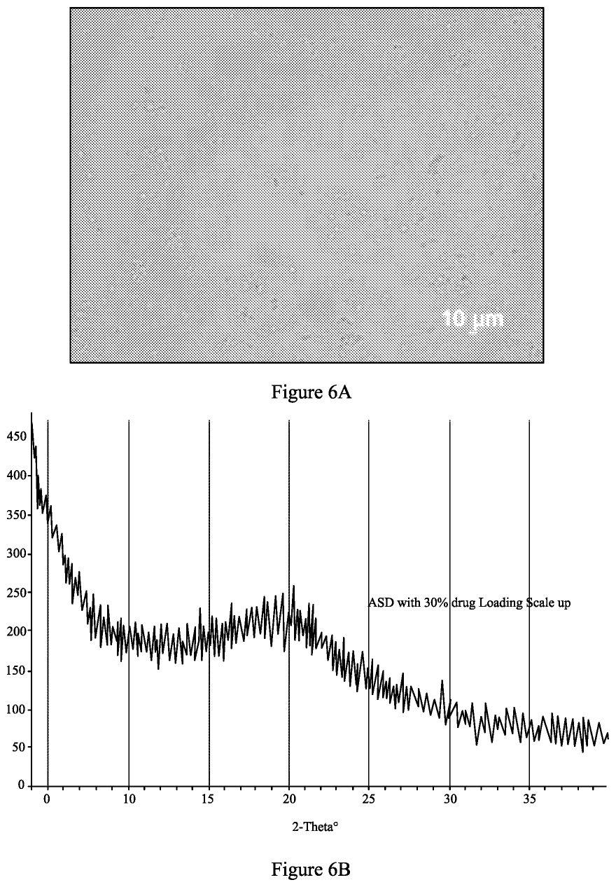

TABLE 9
Details of Soluplus ASDs scale up prepared by nanospray drying

| | | | Drug- | | | |
|---|---|---|---|---|---|---|
| API to Soluplus | | Yield | loading | Purity | | |
| ratio | Appearance | (%) | (%) | (%) | PLM | XRPD |
| 30% API + 70% | White | 59.21 | 31.17 | 99.92 | FIG. 6A | FIG. 6B |
| Soluplus | powder | | | | | |

Capsules Filling with PA-824 ASD

Thirty-five capsules (Size ‘0’, Swedish orange) were filled with each of about 100 mg (±7%) of PA-824 ASD. Three capsules were chosen for DT test. In addition, three capsules (Size ‘0’, White) were each filled with about 100 mg (±7%) of PA-824 ASD as control for DT test. Three capsules (Size ‘0’, White) each were filled with about 100 mg (±7%) of PA-824 ASD and about 30 mg MCC as control for DT test.

Capsules Filling with PA-824 Crushed Tablets

PA-824 Tablets were firstly milled and then passed through 18 mesh to obtain white powder. Thirty-five capsules (Size ‘2’) were filled with each of about 120 mg (±7%) of the white powder. Three capsules were chosen for DT test. Afterwards, the above capsules (Size ‘2’) were replaced by capsules (Size ‘0’) with the same powder. Three capsules were chosen for disintegration (DT) test.

The results of DT test by shown in the Table 10.

TABLE 10
DT test results of two kinds of capsules

| | Capsule | Capsule | Fill weight | |
|---|---|---|---|---|
| Capsule Fill* | size | color | (mg) | DT |
| PA-824 ASD | 0 | Swedish | 100 | 4′30″ |
| | | orange | | |
| PA-824 ASD | 0 | White | 100 | 5′00″ |
| PA-824 ASD and 30 | 0 | White | 130* | 3′30″ |
| mg MCC | | | | |
| PA-824 crushed | 2 | Blue | 120 | 2′30″ |
| tablets | | | | |
| PA-824 crushed | 0 | White | 120 | 2′30″ |
| tablets | | | | |

*Equivalent to 30 mg PA-824

The size 0 white capsules filled with ASD and MCC or with crushed tablets were chosen as final products. Thirty units of each capsules were filled into HDPE bottles.

### Example 1, Part E

### Preparation of ASDs by Hot Melt Extrusion (HME)

Soluplus and HPMC-ASLF were selected for making ASDs. API to polymer ratio of 3:7 (w/w) was used for ASD preparation. To make ASDs, about 1.2 g of the compound and 2.8 g of corresponding polymers were weighed and mixed by vortex mixer. Then the mixture was added into Pharma Mini HME. The instrument was pre-heated to 170° C., the screw speed was set at 100 rpm. Finally, 2.4 g (PA-824:Soluplus 3:7) HME ASD and 2.6 g (PA-824:HPMC-ASLF 3:7) HME ASD was obtained. The ASD products were milled with grinder. The ASDs were characterized by appearance, PLM, mDSC, TGA, XRPD and HPLC (Tables 11 and 12).

TABLE 11
Purity and drug loading results of HME ASDs

| Sample | Purity | Drug load | Amount |
|---|---|---|---|
| PA-824 + Soluplus (3/7, w/w) | 100% | 31.50% | 2.4 g |
| PA-824 + HPMC-ASLF(3/7, w/w) | 100% | 30.23% | 2.6 g |

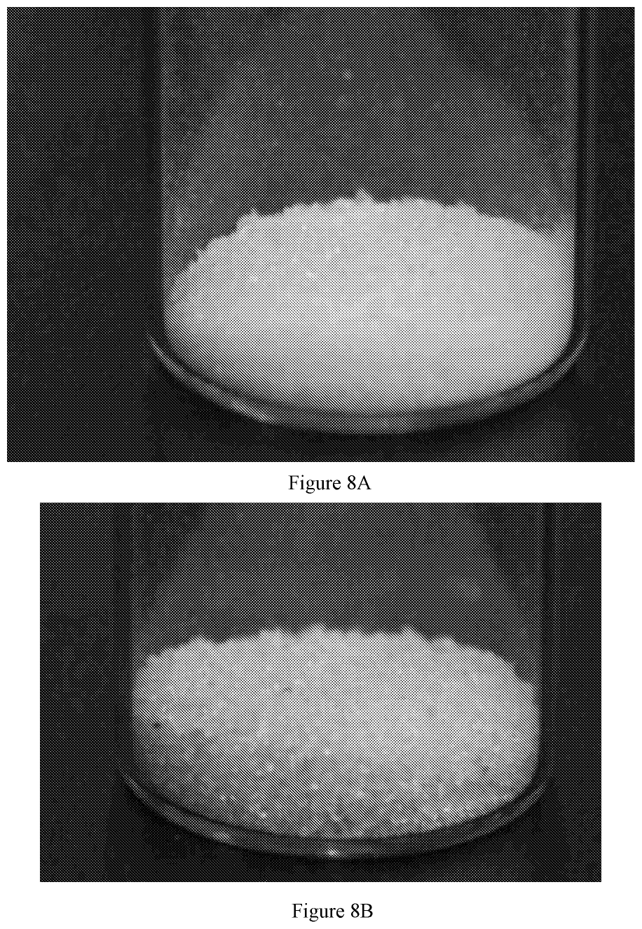

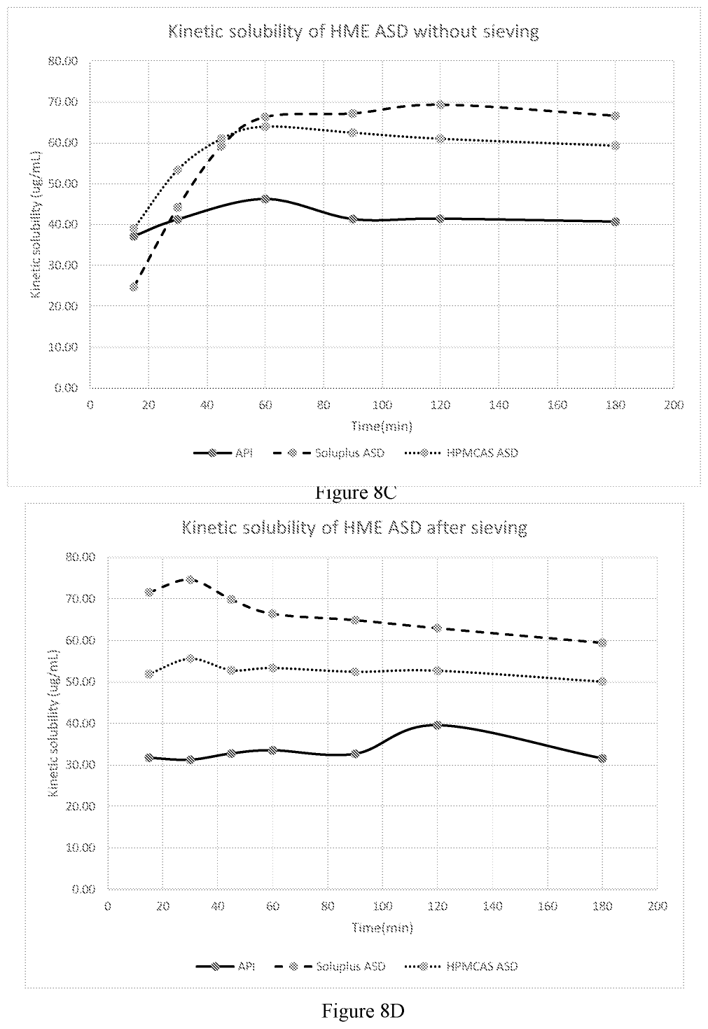

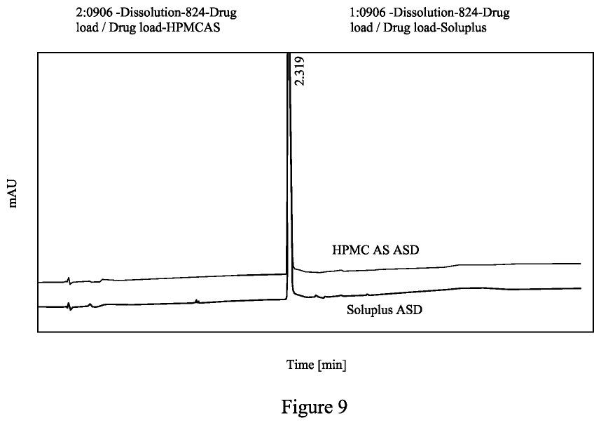

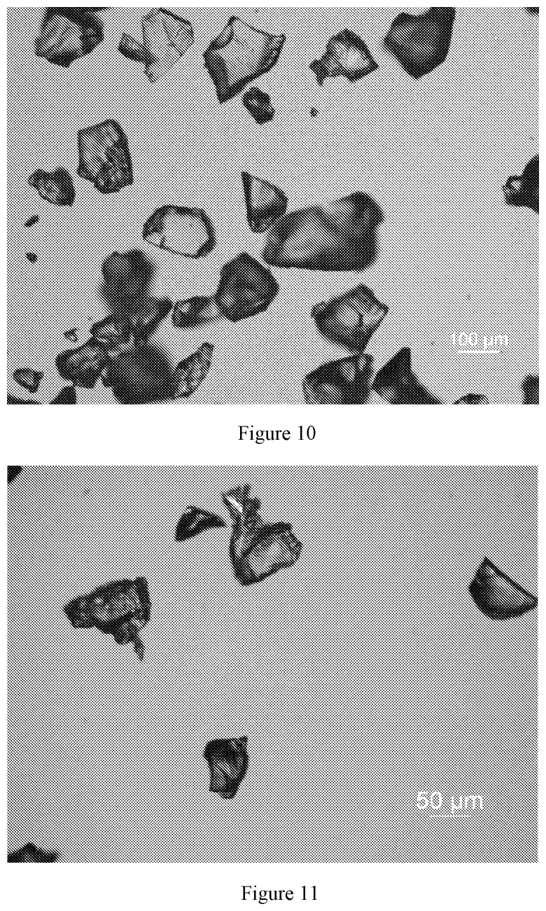

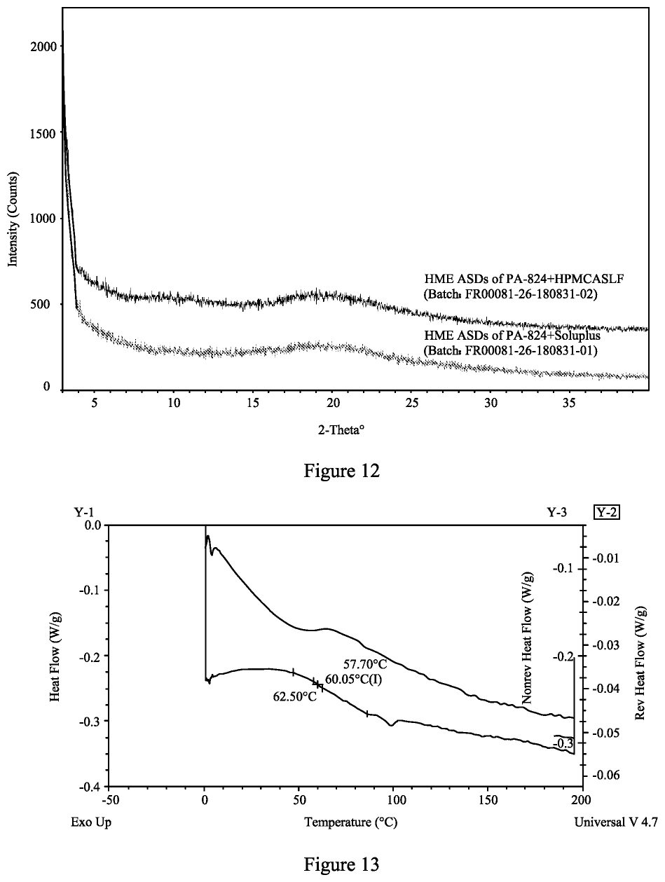

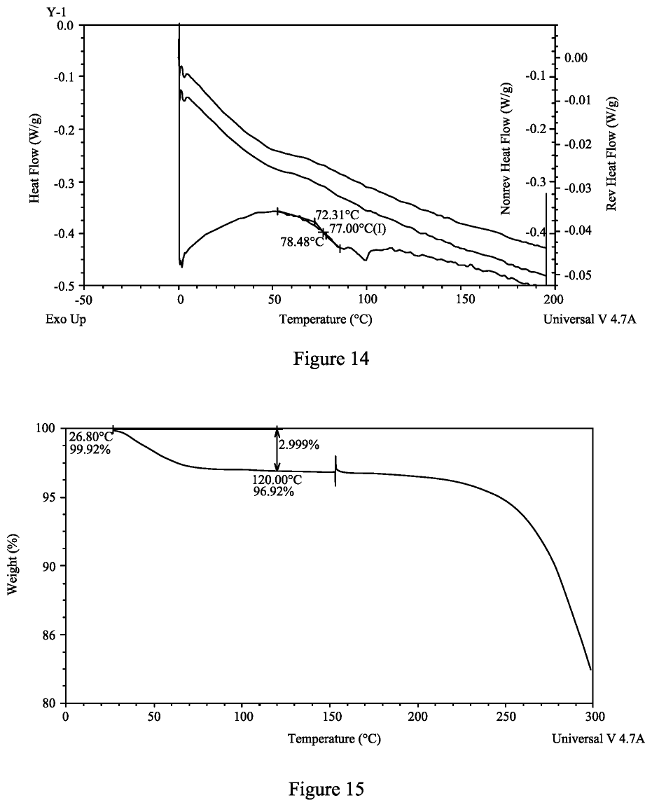

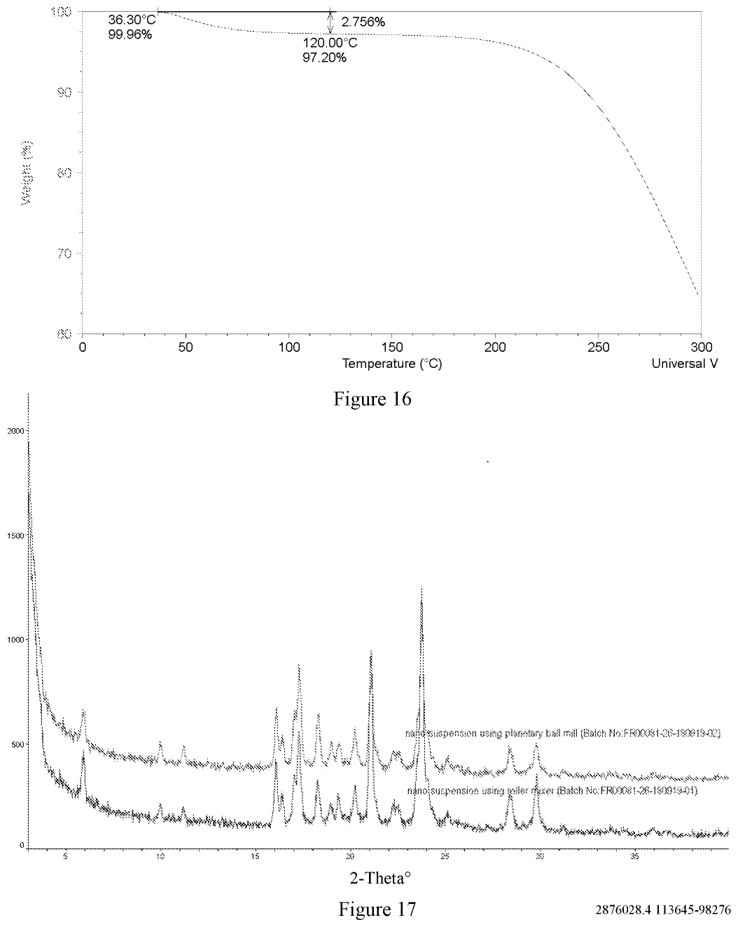

TABLE 12
Characterization of HME ASDs

| HME ASD | Appearance | PLM | XRPD | (m)DSC | TGA | HPLC |
|---|---|---|---|---|---|---|
| Soluplus ASD | FIG. 8A | FIG. 10 | FIG. 12 | FIG. 13 | FIG. 15 | FIG. 9 |
| HPMC ASLF | FIG. 8B | FIG. 11 | FIG. 12 | FIG. 14 | FIG. 16 | FIG. 9 |
| ASD | | | | | | |

### Kinetic Solubility Test of HME ASDs in FaSSIF

10 mL of FaSSIF was added into 4 vials containing each ASD (equivalent to 5mg API) prepared by Hot melt extrusion, respectively, then the suspensions were shaken using a thermomixer at 37° C. under 100 rpm, 300 μL of suspensions were withdrawn at 15, 30, 45, 60, 90, 120 and 180 mins. The samples of suspensions were centrifuged at 14000 rpm for 10 min. The supernatants were diluted with the diluent (ACN/water, 50/50) and analyzed by UPLC. Tables 13 shows the kinetic solubility results of ASDs in FaSSIF. From the data, it was noted that HME was able to improve solubility of PA-824 in FaSSIF.

TABLE 13
Kinetic solubility results of HME ASDs in FaSSIF at 37° C.

| | Solubility (μg/mL) | | | | | | |
|---|---|---|---|---|---|---|---|
| ASD | 15 min | 30 min | 45 min | 60 min | 90 min | 120 min | 180 min |
| API | 37.16 | 41.26 | — | 46.25 | 41.33 | 41.43 | 40.71 |
| 30% API + 70% Soluplus | 24.79 | 44.18 | 59.22 | 66.30 | 67.17 | 69.30 | 66.60 |
| 30% API + 70% HPMC- | 39.05 | 53.39 | 60.96 | 63.94 | 62.44 | 60.97 | 59.27 |
| AS LF | | | | | | | |

The kinetic solubility of HME ASD without sieving is shown in FIG. 8C. The milled ASD products were sieved with 60 mesh sieve, and kinetic solubility test was performed with sieved ASD products as well (Table 14). The kinetic solubility of HME ASD after sieving is shown in FIG. 8D.

TABLE 14
Kinetic solubility results of HME ASDs after sieving in FaSSIF at 37° C.

| | Solubility (μg/mL) | | | | | | |
|---|---|---|---|---|---|---|---|
| ASD | 15 min | 30 min | 45 min | 60 min | 90 min | 120 min | 180 min |
| API | 31.72 | 31.23 | 32.76 | 33.53 | 32.65 | 39.58 | 31.55 |
| 30% API + 70% Soluplus | 71.55 | 74.61 | 69.86 | 66.38 | 64.86 | 62.92 | 59.38 |
| 30% API + 70% HPMC- | 51.89 | 55.58 | 52.81 | 53.34 | 52.43 | 52.69 | 50.04 |
| AS LF | | | | | | | |

### Stability Test for ASDs by HME

About 10 mg ASDs was weighed into 3 vials and placed at 4° C. (close), 25° C./60% RH (close), 40° C./75% RH (close) oven, after 1 week, sample was tested by UPLC to check purity (Table 15).

TABLE 15
Stability data of ASDs at 3 conditions after 1 week (Purity)

| Condition | Time point | Purity (%) | Observation |
|---|---|---|---|
| 4° C. (close) | 1 week | 100 | White powder |
| 25° C./60% RH (close) | | 100 | White powder |
| 40° C.75% RH (close) | | 100 | White powder |
| 4° C. (close) | | 100 | White powder |
| 25° C./60% RH (close) | | 100 | White powder |
| 40° C.75% RH (close) | | 100 | White powder |

### Example 1, Part F

### Preparation of ASDs by Nano Suspension

Vehicles 1 to 4 used for preparation of PA-824 ASD by nano suspension are listed in Table 16.

TABLE 16
Vehicles Used for Preparation of PA-824 ASD by Nano suspension

| Vehicle | Composition |
|---|---|
| Vehicle 1 | 2% PVP K12 and 0.05% Tween 80 in water (w/v) |
| Vehicle 2 | 2% Poloxamer 188 and 0.05% Tween 80 in water (w/v) |
| Vehicle 3 | 0.5% HPMC E5 and 0.05% Tween 80 in water (w/v) |
| Vehicle 4 | 2% Soluplus and 0.05% Tween 80 in water (w/v) |

### Preparation of Vehicle 1:

About 0.05 g of Tween 80 and 2 g of PVP K12 were weighed into a 100 mL volumetric flask, and about 90 mL purified water was added to dissolve. The powder was stirred overnight until completely dissolved. The volume was made up to 100 mL with purified water at room temperature.

### Preparation of Vehicle 2:

About 0.05 g of Tween 80 and 2 g of Poloxamer 188 were weighed into a 100 mL volumetric flask, and about 90 mL purified water was added to dissolve. The powder was stirred overnight until completely dissolved. The volume was made up to 100 mL with purified water at room temperature.

### Preparation of Vehicle 3:

About 0.05 g of Tween 80 and 0.5 g of HPMC E5 were weighed into a 100 mL volumetric flask, and about 90 mL purified water was added to dissolve. The powder was stirred overnight until completely dissolved. The volume was made up to 100 mL with purified water at room temperature.

### Preparation of Vehicle 4:

About 0.05 g of Tween 80 and 2 g of Soluplus were weighed into a 100 mL volumetric flask, and about 90 mL purified water was added to dissolve. The powder was stirred overnight until completely dissolved. The volume was made up to 100 mL with purified water at room temperature.

Note: the weight of the reagents illustrated in the procedure above indicates the standard method from the USP or other literatures. The actual weighing data may vary in a reasonable range.

### Preparation of ASD by Nano Suspension Using Planetary Ball Mill

A certain amount of the compound was weighed into four 12-mL stainless steel jars, and then mill beads and each vehicle was added (1:4). Each suspension was milled for 7 hours in a planetary ball mill. The particle size of suspensions were recorded using a Zeta Potential and Particle Sizer (ZPPS) (Table 17).

TABLE 17
Particle size for PA-824 ASD Nano suspension after
milling for 7 hours in a Planetary Ball Mill

| | | Target | | |
|---|---|---|---|---|
| | | concen- | Particle size | |
| No. | Sample No. | tration | D50 (nm) | D90 (nm) |
| 1 | 2% PVP K12 and 0.05% | 30 mg/ml | 1519.6 | 4035.7 |
| | Tween 80 in water (w/v) | | | |
| 2 | 2% Poloxamer 188 and | | 2678.8 | 7376.8 |
| | 0.05% Tween 80 in water | | | |
| | (w/v) | | | |
| 3 | 0.5% HPMC E5 and | | 1741.3 | 4430.2 |
| | 0.05% Tween 80 in water | | | |
| | (w/v) | | | |
| 4 | 2% Soluplus and 0.05% | | 2437.3 | 7593.4 |
| | Tween 80 in water (w/v) | | | |

### Example 1, Part G

### Preparation of ASD by Nano Suspension Using Roller Mill

A certain amount of the compound was weighed into four 20-mL small plastic bottles, and then mill beads (0.5 mm) and one vehicle was added (1:4) to each bottle. Each suspension was milled for 6 days in a roller mill. The particle size distributions of the suspensions were recorded by ZPPS (Table 18).

Since the nanosuspensions in vehicle 3 (0.5% HPMC E5 and 0.05% Tween 80 in water) with both planetary mill and roller mill had the smallest particle size distribution, the nanosuspensions prepared in this vehicle were characterized by XRPD prior to lyophilization (FIG. 17).

TABLE 18
Particle size for PA-824 ASD Nano suspension
after milling for 6 days in a Roller Mill

| | Target | | | |
|---|---|---|---|---|
| | conc. | | Particle size | |
| No. | mg/mL | Vehicle | D50 (nm) | D90 (nm) |
| 1 | 40 mg/ml | 2% PVP K12 and 0.05% | 4181.6 | 10930.6 |
| | | Tween 80 in water (w/v) | | |
| 2 | | 2% Poloxamer 188 and 0.05% | 11052.8 | 56951.4 |
| | | Tween 80 in water (w/v) | | |
| 3 | | 0.5% HPMC E5 and 0.05% | 1649.5 | 4482.7 |
| | | Tween 80 in water (w/v) | | |
| 4 | | 2% Soluplus and 0.05% | 2139.1 | 5667.3 |
| | | Tween 80 in water (w/v) | | |

### Example 1, Part H

### Lyophilization of Nano Suspension and Evaluation

About 2 mL of each nano suspension prepared in Vehicle 3 (HPMC E5) using roller mill and planetary ball mill was added into two lyophilization vials. The 2 vials with 2 mL of each were put into the freezing dryer, and the temperature probe was inserted into one of the vials with each nanosuspension. The lyophilized samples were analyzed for drug load by HPLC (Table 19). About 3 mg of each lyophilized sample was weighed into vials, then, 1 mL water was added, the dispersibility was observed and tested by particle size distribution analyzer (Table 19). The lyophilized products show good redispersibility.

TABLE 19
Particle size and drug load of Lyophilized PA-824
Nano suspension Prepared in Vehicle 3 (HPMC E5)

| | | Particle size | |
|---|---|---|---|
| Sample No. | Drug load | D50 (nm) | D90 (nm) |
| Redispersibility of lyophilized | 79.21% | 1597.1 | 4541.2 |
| powder (roller mill) | | | |
| Redispersibility of lyophilized | 87.28% | 1788.4 | 6035.5 |
| powder (planetary ball mill) | | | |

### Kinetic Solubility Test of Nano Suspension After Lyophilization in FaSSIF

10 mL of FaSSIF was added into 2 vials containing 2 batches of nano suspension after Lyophilization (equivalent to 2 mg API), then the suspensions were shaken using a thermomixer at 37° C. and 100 rpm, 300 μL of suspensions were withdrawn at 15, 30, 45, 60, 90, 120 and 180 mins. The samples of suspensions were centrifuged at 14000 rpm for 10 min. The supernatants were diluted with the diluent (ACN/water, 50/50) and analyzed by UPLC. Table 20 shows the kinetic solubility results of nano suspension after lyophilization in FaSSIF. From the data, it was noted that Nano suspension cannot improve solubility of PA-824 in FASSIF.

TABLE 20
Kinetic solubility results of nano suspension
after Lyophilization in FaSSIF at 37° C.

| | Solubility (μg/mL) | | | | | | |
|---|---|---|---|---|---|---|---|
| Nano suspension | 15 min | 30 min | 45 min | 60 min | 90 min | 120 min | 180 min |
| API (raw material) | 31.72 | 31.23 | 32.76 | 33.53 | 32.65 | 39.58 | 31.55 |
| Lyophilized powder | 20.49 | 20.91 | 21.73 | 22.85 | 24.08 | 26.85 | 27.66 |
| (roller mixer) | | | | | | | |
| Lyophilized powder | 18.15 | 18.21 | 18.53 | 19.48 | 20.87 | 21.90 | 23.98 |
| (planetary ball mill) | | | | | | | |

### Stability Test for Nano Suspension After Lyophilization

1. About 10 mg 2 batches of nano suspension after Lyophilization were weighed into 2 vials and placed at 4° C. refrigerator (open), after 10 days, the samples were tested by XRPD, purity and particle size.
2. About 10 mg 2 batches of nano suspension after Lyophilization were weighed into 2 vials and placed at 25° C./60% RH (open), after 10 days, the sample were tested by XRPD, purity and particle size.
3. About 10 mg 2 batches of nano suspension after Lyophilization were weighed into 2 vials and placed at 40° C./75% RH (open), after 10 days, the sample were tested by XRPD, purity and particle size.

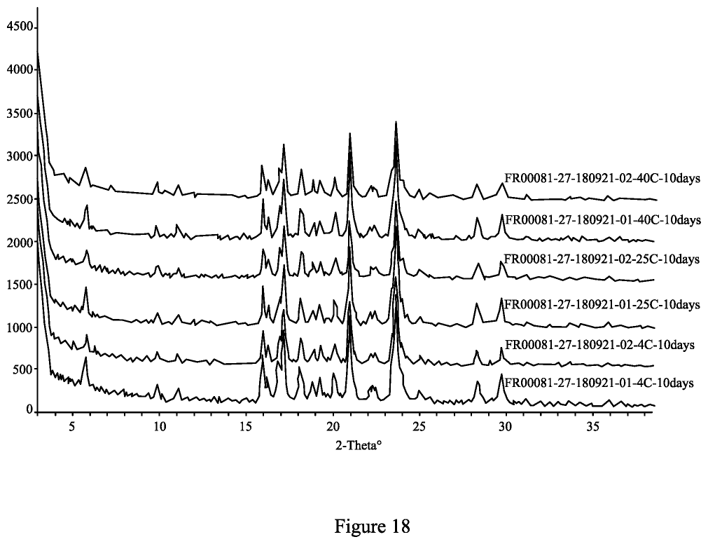

The appearance and purity data for the stability samples are presented in Table 21 and the particle size data are presented in Table 22. The XRPD results for the stability samples are presented in FIG. 18.

TABLE 21
Stability results of purity of lyophilized
powder under various stress conditions

| Samples | Condition | Time point | | Purity (%) | Observation |
|---|---|---|---|---|---|
| 1 | 4° C. (open) | 1 | day | 100 | White powder |
| (roller | | 10 | days | 100 | White powder |
| mixer) | 25° C./60% RH | 1 | day | 100 | White powder |
| | (open) | 10 | days | 100 | White powder |
| | 40° C. 75% RH | 1 | day | 100 | White powder |
| | (open) | 10 | days | 100 | White powder |
| 2 | 4° C. (open) | 1 | day | 100 | White powder |
| (planetary | | 10 | days | 100 | White powder |
| ball mill) | 25° C./60% RH | 1 | day | 100 | White powder |
| | (open) | 10 | days | 100 | White powder |
| | 40° C. 75% RH | 1 | day | 100 | White powder |
| | (open) | 10 | days | 100 | White powder |

TABLE 22
Particle size for nano suspension after Lyophilization
after 10 days under various stress conditions

| | | Particle size | |
|---|---|---|---|
| Sample | Condition | D50 (nm) | D90 (nm) |
| 1 | 4° C. (open) | 1745.8 | 4842.1 |
| (roller mixer) | 25° C./60% RH (open) | 790.0 | 1802.1 |
| | 40° C./75% RH (open) | 1795.6 | 4844.6 |
| 2 | 4° C. (open) | 1537.4 | 3930.6 |
| (planetary ball mill) | 25° C./60% RH (open) | 1863.6 | 5199.7 |
| | 40° C./75% RH (open) | 1010.8 | 3107.3 |

### Example 2

### Amorphous Solid Dispersion Matrix Studies

### Preparation of Standard Solutions for PA-824

For the estimation of the re-dissolved PA-824 from micro-evaporative dispersion samples, a UV-spectrophotometric assay was developed. Standard solutions of PA-824 were prepared for the purposes of running standard curves. A 250 μg/mL solution of drug was prepared in Methanol. From this stock solution, 5 standards were prepared using 1:2 serial dilutions having concentrations of 3.90625-62.5 μg/mL.

### Micro-Evaporation Studies

Various dispersion formulations were studied. Combinations of API and candidate polymer solutions are prepared in micro-centrifuge tubes. The solvent is dried from the samples in a vacuum concentrator. Dry samples are reconstituted in phosphate buffer pH 6.8 and mixed for 4, 10, 30, and 60-minute time intervals. Re-dissolution behavior of API is measured with UV spectrometry to determine performance of polymer matrix. The polymer matrices and composition screened are shown in Table 23.

TABLE 23
ASD Formulations Screened with Micro-Evaporation Studies

| Formulation | Formulation Composition |
|---|---|
| Matrix 1 | API - HPMCAS-L (30-70) |
| Matrix 2 | API - Soluplus (30-70) |
| Matrix 3 | API - Kollidon VA 64 (30-70) |
| Matrix 4 | API - Soluplus - TPGS (30-60- |
| | 10) |
| Matrix 5 | API - Soluplus - Poloxamer 407 |
| | (30-60-10) |
| Matrix 6 | API - Soluplus - SLS (30-60- |
| | 10) |
| Matrix 7 | API - Kollidon VA 64 - |
| | Soluplus (30-56-14) |
| Matrix 8 | API - Kollidon VA 64 - |
| | Soluplus (30-14-56) |
| Matrix 9 | API - Kollidon VA 64 - |
| | Soluplus (30-35-35) |

The following solutions were prepared for sample preparation of binary or tertiary combinations of API, polymer and surfactants:
1. API Solution at 1 mg/mL by weighing 100 mg of API into 100 mL volumetric flask. Added 100 mL of MeOH. Sonicated to dissolve.
2. HPMCAS-L Solution at 20 mg/mL by weighing 200 mg into a scintillation vial. Added 10 mL of MeOH. Sonicated to dissolve.
3. Kollidon VA64 Solution at 20 mg/mL by weighing 200 mg into a scintillation vial. Added 10 mL of MeOH. Sonicated to dissolve.
4. Soluplus Solution at 20 mg/mL by weighing 200 mg into a scintillation vial. Added 10 mL of MeOH. Sonicated to dissolve.
5. D-α-tocopheryl polyethylene glycol succinate (TPGS) Solution at 10 mg/mL by weighing 100 mg into a scintillation vial. Added 10 mL of MeOH. Sonicated to dissolve.

6. Poloxamer 407 Solution at 10 mg/mL by weighing 100 mg into a scintillation vial. Added 10 mL of MeOH. Sonicated to dissolve.
7. Sodium dodecyl sulfate (SLS) Solution at 10 mg/mL by weighing 100 mg into a scintillation vial. Added 10 mL of MeOH. Sonicated to dissolve.
8. SLS Solution—10 mg/mL (in EtOH) by weighing 99.6 mg SLS into a scintillation vial. Added 10 mL of EtOH. Sonicated to dissolve.
9. SLS Solution—5 mg/mL (in EtOH) by weighing 99.6 mg SLS into a scintillation vial. Added 20 mL of EtOH. Sonicated to dissolve.

### Sample Preparation for Micro-Evaporation Screening Studies

In 1.5 mL centrifuge tubes, individual samples were prepared for each of the following time points: 4 min, 10 min, 30 min, 60 min. API alone samples were prepared as a control with a nominal concentration of 1000 μg/mL. Matrix samples without API were prepared as blanks. API+Matrix samples were prepared in duplicate for each timepoint and according to the formulation composition in Table 23. The micro-evaporation screening studies were run in four different parts; the respective sample preparation and volume of each matrix component added to each tube for each part of the study is listed below.

### Part I: Experiments for Comparison

PA-824 API alone control: 1000 μL of API solution
Matrix 1 blank: 166.6 μL of HPMCAS-L solution
Matrix 2 blank: 166.6 μL of Soluplus solution
Matrix 3 blank: 166.6 μL of Kollidon VA64 solution
API+Matrix 1: 1000 μL of API solution+166.6 μL of HPMCAS-L solution
API+Matrix 2: 1000 μL of API solution+166.6 μL of Soluplus solution
API+Matrix 3: 1000 μL of API solution+166.6 μL of Kollidon VA64 solution
   Part II: Polymer Matrix with Surfactant
PA-824 API alone control: 1000 μL of API solution
Matrix 4 blank: 100 μL of Soluplus solution+33 μL of TPGS solution
Matrix 5 blank: 100 μL of Soluplus solution+33 μL of Poloxamer 407 solution
Matrix 6 blank: 100 μL of Soluplus solution+33 μL of SLS solution
API+Matrix 4: 1000 L of API solution+100 μL of Soluplus solution+33 μL of TPGS solution
API+Matrix 5: 1000 μL of API solution+100 μL of Soluplus solution+33 μL of Poloxamer 407 solution
API+Matrix 6: 1000 μL of API solution+100 μL of Soluplus solution+33 μL of SLS solution

### Part III: Surfactant Controls

PA-824 API alone control: 1000 μL of API solution
Surfactant in Matrix 4 blank: 33 μL of TPGS
Surfactant in Matrix 5 blank: 33 μL of Poloxamer 407 solution
Surfactant in Matrix 6 blank: 66 μL of SLS solution
API+Surfactant in Matrix 4: 1000 μL of API solution+33 μL of TPGS solution
API+Surfactant in Matrix 5: 1000 μL of API solution+33 μL of Poloxamer 407 solution
API+Surfactant in Matrix 6: 1000 μL of API solution+66 μL of SLS solution

### Part IV: Two Polymer Matrix

PA-824 API alone control: 1000 μL of API solution
Matrix 7 blank: 93 μL of Kollidon VA 64 solution+23 μL Soluplus solution
Matrix 8 blank: 23 μL of Kollidon VA 64 solution+93 μL Soluplus solution
Matrix 9 blank: 58 μL of Kollidon VA 64 solution+58 μL Soluplus solution
API+Matrix 7: 1000 μL of API solution+93 μL of Kollidon VA 64 solution+23 μL Soluplus solution
API+Matrix 8: 1000 μL of API solution+23 μL of Kollidon VA 64 solution+93 μL Soluplus solution
API+Matrix 9: 1000 μL of API solution+58 μL of Kollidon VA 64 solution+58 μL Soluplus solution.

### Reconstitution and UV Analysis for Micro-Evaporation Screening Studies

All centrifuge tubes were placed in vacuum concentrator at 37° C. on Manual mode, RC on for 45 minutes to an hour. Once all the solvent was evaporated, 1 mL of Phosphate buffer pH 6.8 was added to each tube. All tubes were placed on the disruptor for 4 min, 10 min, 30 min and 60 min. After each timepoint on disruptor is complete, tubes were placed in microcentrifuge for 4 minutes at 15000 rpm. The supernatant from each tube was removed. An aliquot of the supernatant was diluted 1:10 in Phosphate buffer pH 6.8 and added to individual wells of 96-well plate. The 96-well plate was analyzed on the UV plate reader at API lambda max: 320 nm.

### DSC Testing of Physical Mixtures of API and Polymer Matrices

A physical mixture of API with polymers and polymer-surfactant combinations were loaded onto a DSC and evaluated for miscibility with the matrix. The physical mixtures were loaded onto a DSC pan with 3-10 mg of sample. A heat-cool-heat cycle was used for physical mixtures of API, polymer and surfactant.

### Sample Preparation for DSC Screening of Physical Mixtures of API and Polymer Matrices

Physical mixtures of the API with some of the matrices listed in Table 23 were weighed into in 1.5 mL microcentrifuge tubes. Blank controls of each matrix component were also weighed into 1.5 mL centrifuge tubes. Each tube was vortexed for 10 seconds. For samples containing TPGS, the TPGS was first mixed with Soluplus in a mortar and pestle due to its waxy consistency. Then the sample vortexed for 10 seconds on a vortexer. The amount of API and matrix component weighed into each tube is listed below.

### Control Matrices:

Matrix 1 API+Soluplus (30:70): 30.084 mg of API+70.094 mg of Soluplus
Matrix 2: API+HPMCAS-L (30:70): 30.106 mg of API+70.074 mg of HPMCAS-LG
Matrix 3: API+Kollidon VA64 (30:70): 30.097 mg of API+70.028 mg of Kollidon VA64
   Polymer Matrices with Surfactant:
Matrix 4 API+Soluplus+TPGS (30:60:10): 30.043 mg of API+60.085 mg of Soluplus+10.559 mg of TPGS.
Matrix 5 API+Soluplus+Poloxamer 407 (30:60:10): 30.054 mg of API+60.090 mg of Soluplus+10.057 mg of Poloxamer 407
Matrix 6 API+Soluplus+SLS (30:60:10): 30.067 mg of API+60.065 mg of Soluplus+10.065 mg of SLS

Blank Controls: HPMCAS-L alone; Kollidon VA64 alone; Soluplus alone; SLS alone; TPGS alone; Poloxamer 407 alone; Soluplus+TPGS (60:10): 59.957 mg of Soluplus+10.023 mg of TPGS; Soluplus+Poloxamer 407 (60:10): 60.124 mg of Soluplus+10.089 mg of Poloxamer 407; Soluplus+SLS (60:10): 60.071 mg of Soluplus+10.061 mg of SLS.

### Results/Discussion

### Miscibility Modeling and Characterization of PA-824

### Theoretical Assessment API and Polymer Miscibility

Polymer matrix choice is driven by maximum miscibility of the API and polymer. It is proposed that polymer selection can be based on the value of difference of the HSP (Hansen solubility parameters) of the API and the polymer, where a value less than 2.0 MPa^{0.5} is preferred. Eudragit L100, L100-55, Kollidon VA64, PVP K30, HPMCAS-M and Soluplus in combination with Kollidon VA64 are investigated for compatibility with API based on this criteria.

### Characterization of API

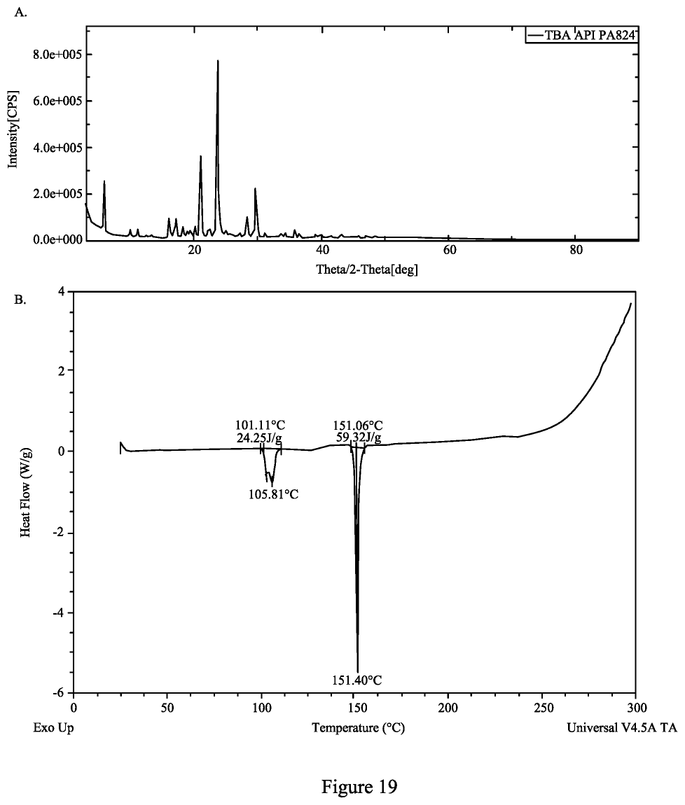

The results for DSC and XRD of PA-824 API are shown in FIG. 19 (A and B). The DSC thermogram for PA-824 API shows two endothermic events; the first endothermic event around 105° C. and the second around 151° C. The first endothermic event around 105° C. corresponds to solid-solid transition of Form I to Form II of the API and is reversible. The second endothermic event around 151° C. corresponds to the melting of Form II and is irreversible.

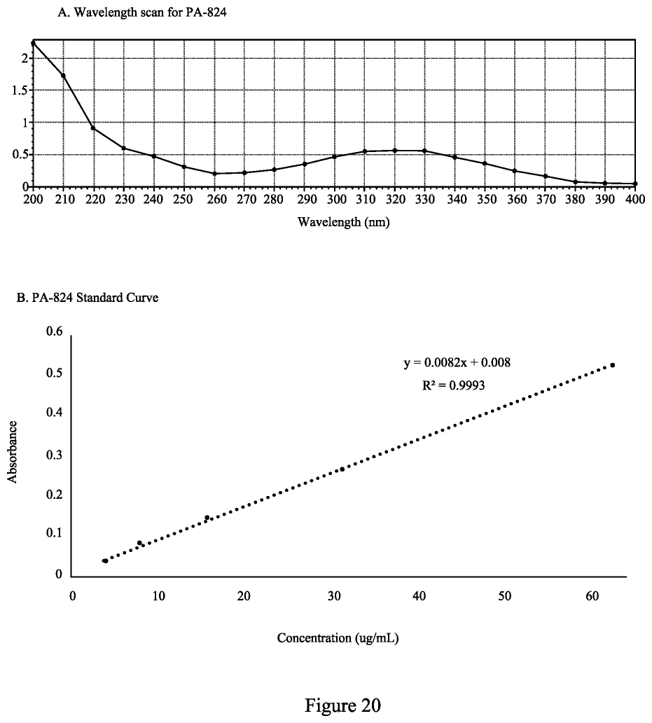

Four different solid-state forms of the API have been identified and characterized. Form I of the API is crystalline, non-solvated and the most thermodynamically stable under ambient temperature and pressure conditions. Form II of the API is crystalline, non-solvated and exists at elevated temperatures only (above 100° C.). Determination of wavelength of maximum absorbance (λmax) for PA-824. The wavelength of maximum absorbance (λmax) of PA-824 was found to be at 320 nm as seen in FIG. 20A.

Table 24 shows absorbance reading for PA-824 standard solutions. FIG. 20B shows the API standard curve with regression equation.

TABLE 24
Absorbance readings for PA-824 standard solutions

| Conc (ug/mL) | Absorbance | Normalized Absorbance |
|---|---|---|
| 0 | 0.049 | 0 |
| 62.5 | 0.57 | 0.521 |
| 31.25 | 0.311 | 0.262 |
| 15.625 | 0.189 | 0.14 |
| 7.8125 | 0.127 | 0.078 |
| 3.90625 | 0.082 | 0.033 |

### Amorphous Solid Dispersion Matrix Screening Studies

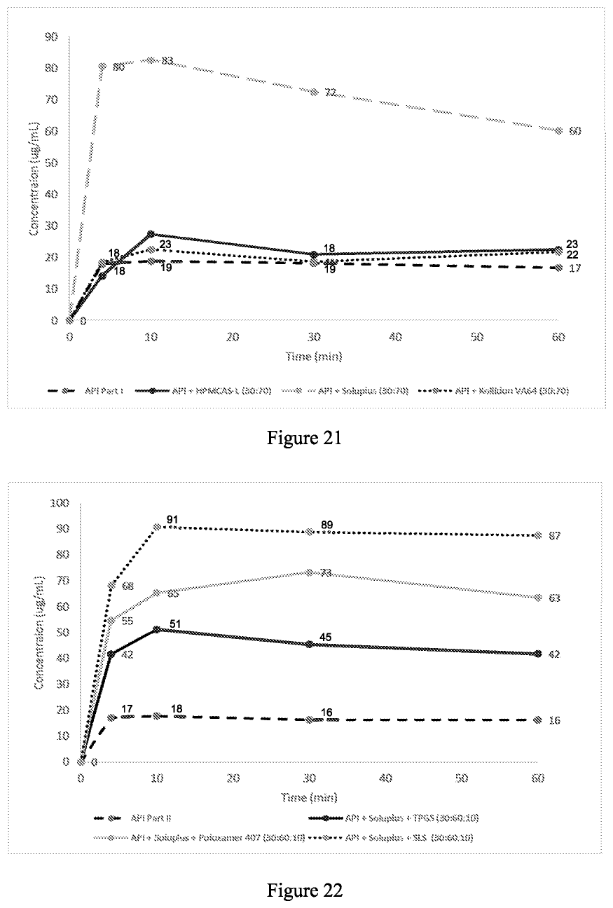

FIGS. 21-24 show the micro evaporation results for Part I-IV of the study, respectively. The API concentration was determined using the API standard curve equation. API+matrix samples were prepared and measured in duplicate at each timepoint.

In Part I, Soluplus and HPMCAS-L containing matrices were included in this study as positive controls, and Kollidon VA64 containing matrix is included as a negative. Part II of this study was run with Soluplus polymer+surfactant test matrices. The surfactants added to the test matrices act as a solubilizer+phase separation inhibitor; thus, were evaluated to see if polymer and surfactant matrices show higher API concentrations and/or persistently higher API concentrations over time. Part III of this study was run with API+surfactant controls (without polymer).

Compared to API alone evaluated in Part I, II, III, and IV, all the API+polymer as well as API+polymer+surfactant matrices showed higher API concentration and thus improved solubilization of API over time. Additionally, the results from this study demonstrate advantages of Soluplus and HMPCAS-L. Indeed, Soluplus showed the highest API solubilization. Kollidon VA 64 had the lowest solubilization of API.

In Part III of this study, the API+TPGS control showed higher API concentration than API+Soluplus+TPGS from Part II. However, API+surfactant (without polymer) is not the most preferred amorphous solid dispersion formulation and thus are not included in the rank ordering of matrices. It should be noted that Part III samples were prepared on a different day than Part I and Part II samples; with fresh API and surfactant stock solutions, which may contribute to some variability in the results for Part II and Part III. The API concentration was calculated from the UV standard curve equation.

Notably from Part II of this study, the addition of surfactants to the API+Soluplus matrix did show a persistent solubilization of the API over a period of 60 minutes. Specifically, the addition of SLS surfactant to the API+Soluplus matrix showed higher redissolution of the API compared to just API+Soluplus matrix. Also, the higher redissolution of the API persisted over of 60 minutes; while the redissolution of the API reduced over time for just API+Soluplus matrix. Thus, it can be concluded the Soluplus+SLS matrix is more stable than the control and other test matrices.

From animal PK studies, the amorphous dispersion API+Soluplus matrix indicated no difference in bioavailability compared to the crystalline tablet formulation. Without being bound by theory, it is contemplated that the inability of amorphous dispersion to improve bioavailability is because the amorphous API administered as spray dried dispersion in animal PK studies recrystallized in the gastrointestinal tract upon oral administration, potentially by phase separation between the API and polymer phases of the dispersion. However, with the results from this example showing the API+Soluplus+SLS matrix has the highest API redissolution that persisted over a period of 60 minutes, it can be concluded that SLS inhibits phase separation between API and polymer phases of the dispersion. The API+Soluplus+SLS amorphous dispersion formulation improves bioavailability which would not have been expected from published literature.

Part IV of this study was conducted after SDD trial 1 and SDD trial 2 showed residual crystallinity (as described below) and the stabilization of the polymer matrix was required in order to prevent recrystallization of the API. A two-polymer system was proposed to stabilize the polymer matrix in which Soluplus would act as a solubilizer. Kollidon VA64 was selected as the primary polymer as this showed good miscibility with the API in DSC screening (as shown above) and from miscibility modeling. Although the two-polymer matrices in this part of the study had lower API concentration than matrices in Part II (polymer+surfactant), the two polymer matrices showed higher redissolution than API+HPMCAS-L and API+Kollidon VA 64 matrices in Part I. This indicates that a two-polymer system may help stabilize the polymer matrix and thus is a formulation for improving bioavailability.

### Micro-Esvaporation Screening Study

FIG. 21 shows the micro-evaporation results for Part I samples. API concentration was improved (higher than API alone) for the three SDD matrices tested. The API+Soluplus matrix showed the highest API concentration; 80 μL/mL and 83 μg/mL at 4 and 10-minute timepoints respectively (a ˜4-fold increase compared to API alone); slight decrease to 60 μg/mL at the 60-minute timepoint. The API+HPMCAS-L and API+Kollidon VA 64 matrices showed a slight increase in concentration compared to API alone.

FIG. 22 shows the micro-evaporation results for Part II samples. API concentration improved (higher than API alone) for the three SDD polymer matrices+surfactant combinations tested. Compared to API+Soluplus matrix from Part I, the addition of SLS to the API+Soluplus matrix shows the API concentration did not decrease overtime, rather was sustained at 87 μg/mL at 60-minutes timepoint. A similar trend is seen with the addition of Poloxamer 407 surfactant to the Soluplus matrix, however, the API concentration was lower compared to the Soluplus+SLS. The addition of TPGS surfactant did not appear to sustain the API concentration out to the 60-minute timepoint.

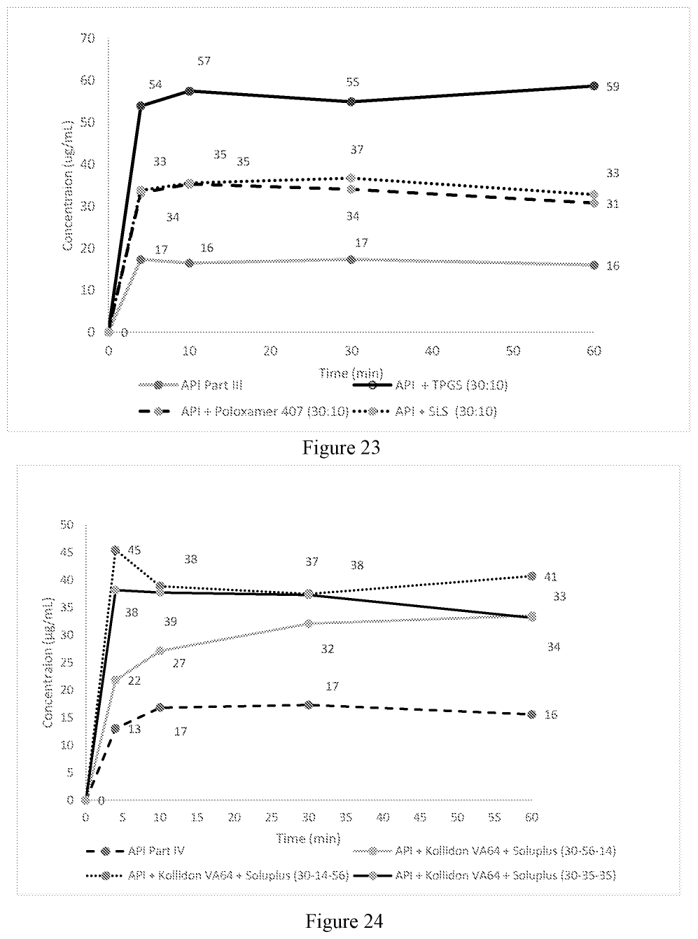

FIG. 23 shows the micro-evaporation results for Part III samples. For API+TPGS surfactant control, API concentration improved compared to API alone; ˜3-fold higher concentration. For Poloxamer 407 and SLS, API concentration also improved compared to API alone, but was ˜2-fold higher.

FIG. 24 shows the micro-evaporation results for Part IV samples (with two-polymer matrices). API+Kollidon VA64+: Soluplus (30:56:14) and (30:35:35) show about 3 fold higher API concentration than API alone.

### DSC Screening of Physical Mixtures of API and Polymer Matrices

The following sections show the resulting DSC thermograms for the samples described earlier in this Example. It should be noted that for the heat-cool-heat runs for all samples other than API alone, an artifact can be seen during the cool run around 65° C. This artifact is from the DSC sensor and is not an actual thermal event.

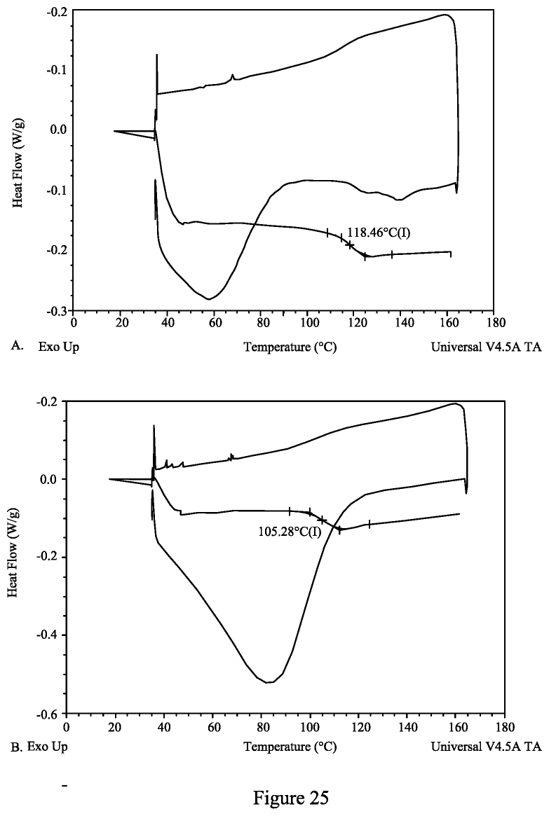

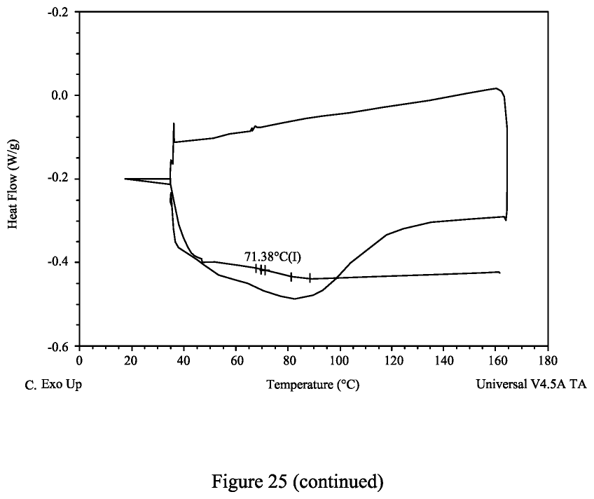

FIG. 25 shows the DSC thermogram for polymer alone blank controls: HPMCAS-L, Kollidon VA 64, and Soluplus. The DSC thermogram for HPMCAS-L shows the glass transition (Tg) for HPMCAS-L at 118.46° C. during the second heat run. The DSC thermogram for Kollidon VA64 shows the glass transition (Tg) for Kollidon VA64 at 105.28° C. during the second heat run. The DSC thermogram for Soluplus shows the glass transition (Tg) for Soluplus at 71.38° C. during the second heat run.

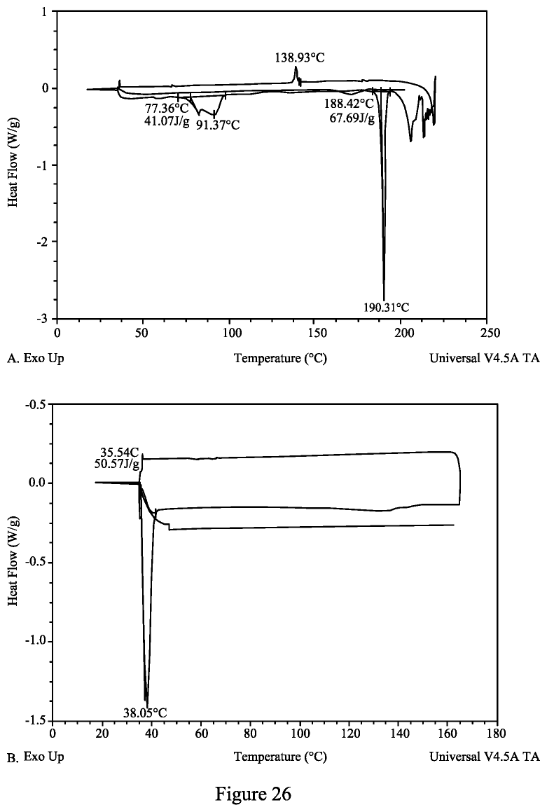

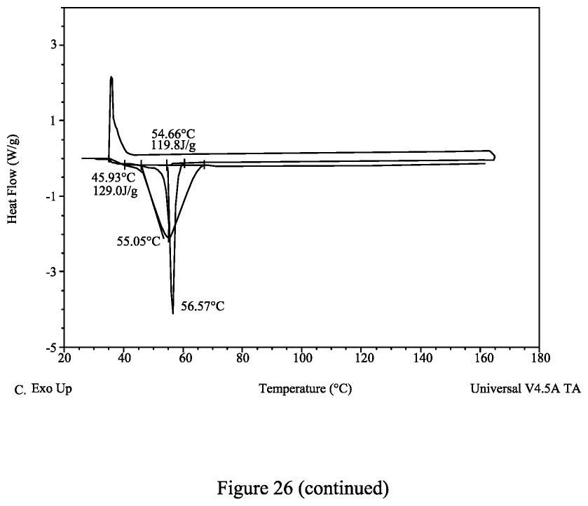

FIG. 26 shows the DSC thermogram for surfactant alone blank controls: SLS, TPGS, and Poloxamer 407 alone. The DSC thermogram for SLS shows an endothermic peak at 91.37° C. and a second sharp endothermic peak at 190.31° C. During the cool run, an exothermic peak is seen at 138.93ºC. The DSC thermogram for TPGS shows a sharp endothermic melting peak at 38.05° C. during the first heat run. The DSC thermogram for Poloxamer 407 shows a sharp endothermic melting peak at 56.57° C. during the first heat run.

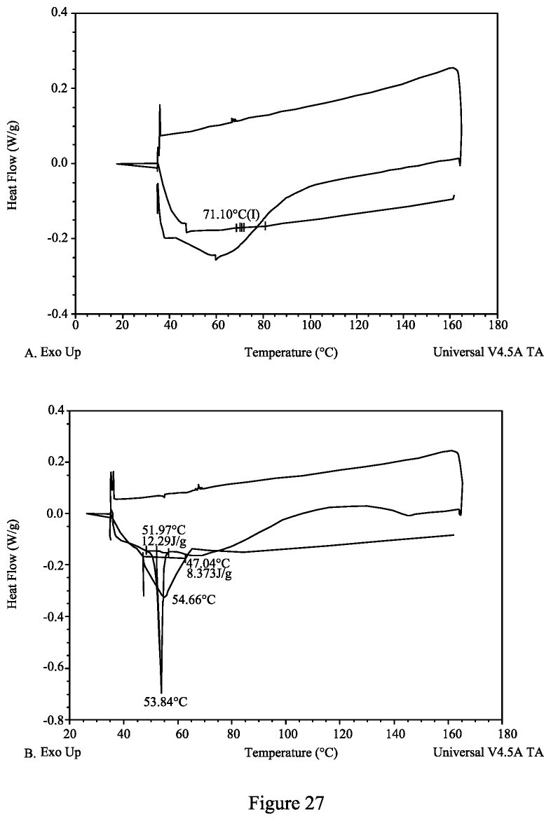

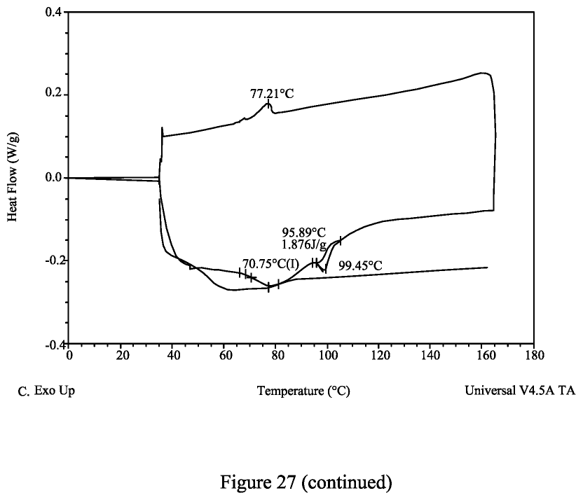

FIG. 27 shows the DSC thermogram for Soluplus+surfactant blank controls: Soluplus+TPGS (60:10), Soluplus+Poloxamer 407 (60:10) and Soluplus+SLS (60:10). The DSC thermogram for Soluplus+TPGS shows a glass transition (Tg) for Soluplus at 71.10° C. during the second heat run. The DSC thermogram for Soluplus+Poloxamer 407 shows a sharp endothermic melting peak for Poloxamer 407 at 53.84° C. during the first heat run. The DSC thermogram for Solupus+SLS shows a glass transition (Tg) at 70.75° C. and an endothermic peak at 99.45° C. during the first heat run. During the cool run, an exothermic peak is seen at 77.21° C.

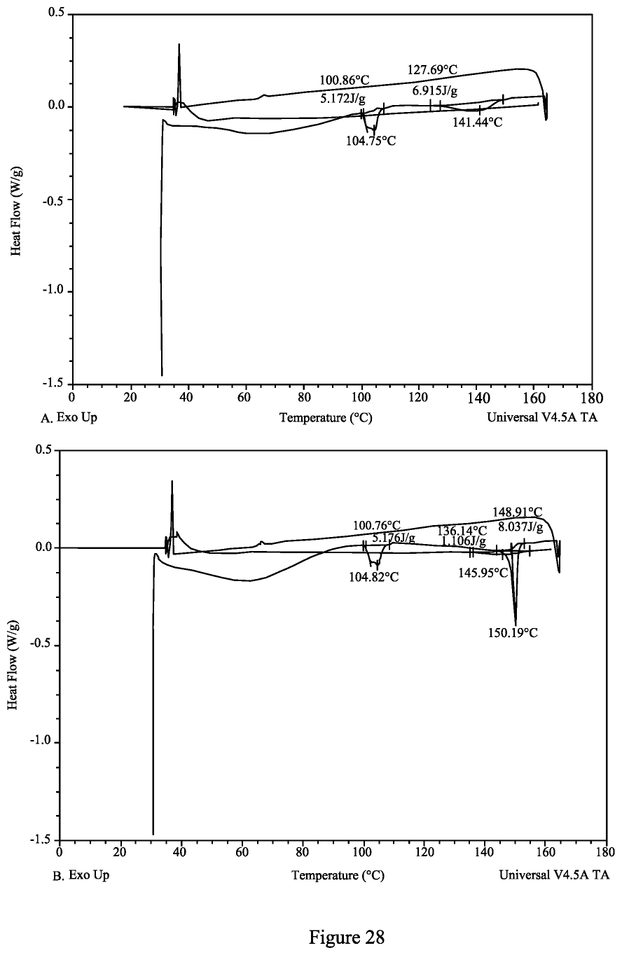

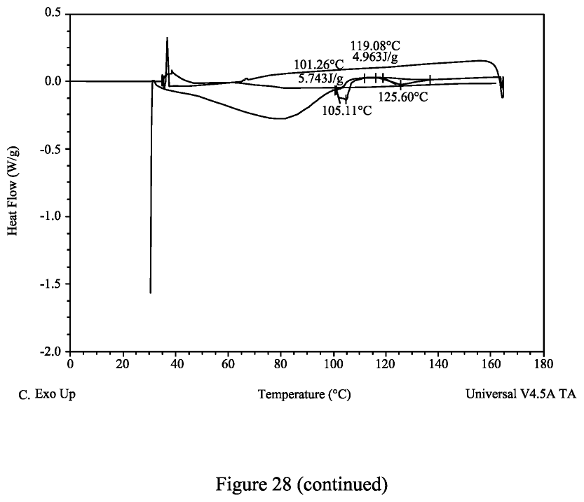

FIG. 28 shows the DSC thermogram for matrices 1, 2 and 3. The DSC thermogram for Matrix 1, API+Soluplus (30:70) shows an endotherm at 104.75° C. and another wide endotherm at 141.44° C. during the first heat run. The absence of the API melting peaks shows that the API is fully dissolved in the matrix and exist as amorphous form. The DSC thermogram for Matrix 2, API+HPMCAS-M (30:70) matrix shows an endotherm at 104.82° C. and 150.19° C. during the first heat run. During the second heat run, there is a small endotherm at 145.95° C. Also, as seen during the second heat run, HPMCAS-L is found to significantly shift the melting peak and reduce the crystallinity of the API in the physical mixture. The DSC thermogram for Matrix 3, API+Kollidon VA64 (30:70) shows an endotherm at 105.11° C. and another wide endotherm at 125.60° C. during the first heat run. The absence of the API melting peaks in the second run shows that the API is fully dissolved in the matrix and exist as amorphous form.

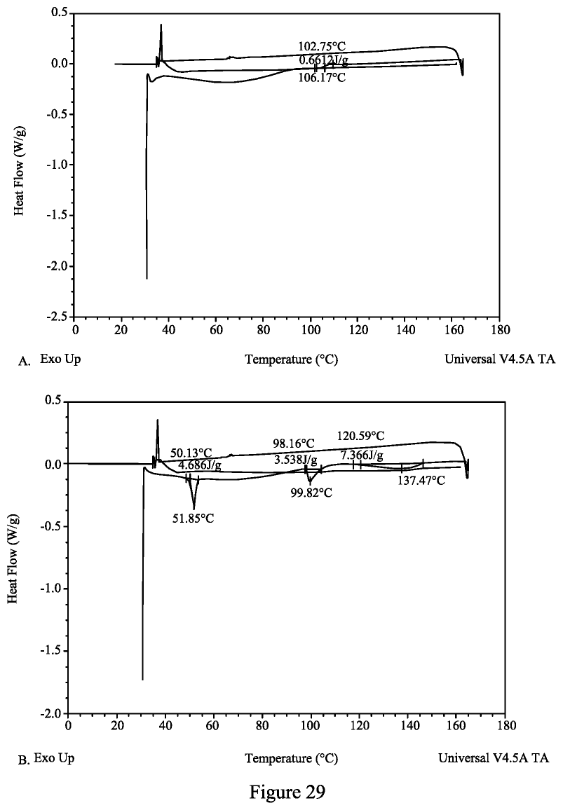

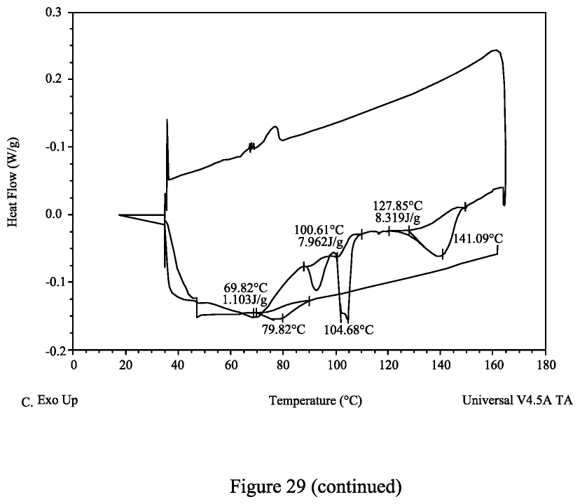

FIG. 29 shows the DSC thermogram for matrices 4, 5, and 6. The DSC thermogram for Matrix 4, API+Soluplus+TPGS (30:60:10) shows a small endotherm at 106.17° C. during the first heat run. The absence of the API melting peaks in the second run shows that the API is fully dissolved in the matrix and exist as amorphous form. The DSC thermogram for Matrix 5, API+Soluplus+Poloxamer 407 (30:60:10) shows an endotherm at 51.85° C., 99.82° C. and 137.47° C. during the first heat run. The absence of the API melting peaks in the second run shows that the API is fully dissolved in the matrix and exist as amorphous form. The DSC thermogram for Matrix 6, API+Soluplus+SLS (30:60:10) shows an endotherm at 104.68° C. and another wide endotherm at 141.09° C.during the first heat run. During the cool run and the second heat run respectively, a crystallization exothermic peak is seen around 75° C. and a wide endotherm is seen at 79.82° C., which could be attributed to recrystallization of the API.

Table 25 below shows a summary of DSC thermogram data extracted from the second heat run of the physical mixtures with API and from the heat ramp for API alone sample. For PA824 API alone, the enthalpy of fusion from the 1st and 2nd peak were combined as the total enthalpy of fusion. Matrices 2 and 4 are substantially amorphous with less than 5% residual crystallinity.

TABLE 25
Summary of Data from Second Heat Run for Physical Mixtures with
API (Samples 1-7) with Polymers, and Soluplus with surfactants

| | | | Second Heat Run | | |
|---|---|---|---|---|---|
| | | | | Enthalpy | |
| Matrix | | Figure | Tm | of Fusion | Crystallinity |
| # | Composition | No. | (° C.) | (J/g) | (%) |
| — | PA824 API | 19 | 1st Peak: 105.81 | 1st Peak: 24.25 | 100% |
| | Alone | | 2nd Peak: 151.40 | 2nd Peak: 59.32 | |
| | (taken from heat | | | Total: 83.57 | |
| | ramp) | | | (1st Peak & 2nd | |
| | | | | Peak combined) | |
| 1 | API + Soluplus | 28 A | — | — | Amorphous |
| | (30:70) | | | | |
| 2 | API + | 28 B | 145.95 | 1.106 | 4.4% |
| | HPMCAS-L | | | | |
| | (30:70) | | | | |
| 3 | API + Kollidon | 28 C | — | — | Amorphous |
| | VA64 (30:70) | | | | |
| 4 | API + Soluplus + | 29 A | — | — | Amorphous |
| | TPGS (30:60:10) | | | | |
| 5 | API + Soluplus + | 29 B | — | — | Amorphous |
| | Poloxamer 407 | | | | |
| | (30:60:10) | | | | |
| 6 | API + Soluplus + | 29 C | 79.82 | 1.103 | 4.4% |
| | SLS (30:60:10) | | | | |

The DSC thermograms for API+Soluplus and API+Kolidon VA64 physical mixtures showed an absence of the API melting peaks during the second heat runs, which indicates the API fully dissolved in the matrix and exists as an amorphous form. The DSC thermogram for API+HPMCAS-L physical mixture is found to significantly shift the API melting peak (for Form II; 151.40° C.) and reduce the crystallinity of the API in the mixture; 4.4% crystallinity is calculated from enthalpy of fusion.

The DSC thermogram for API+Soluplus+Poloxamer 407 and API+Soluplus+TPGS physical mixtures showed an absence of the API melting peaks in the second heat runs, which indicates the API is fully dissolved in the matrix and exists as an amorphous form. The DSC thermogram for API+Soluplus+SLS has an endothermic peak around 79° C., which is a significant shift in the API melting peak and reduction in crystallinity of API in the physical mixture. 4.4% crystallinity is calculated form enthalpy of fusion.

Based on the resulting DSC data from DSC of the API alone and the physical mixtures with the API, the PA824 API demonstrates good miscibility with all three of the polymer matrices; with Soluplus and Kollidon VA64 matrices showing slightly better miscibility than the HPMCAS-L matrix. The results demonstrate that Soluplus provides superior results in amorphous solid dispersion (ASD) formulations. Additionally, based on the data for the physical mixtures with the API and the melting behavior of the API alone, it is contemplated that preparing ASD formulations using Hot Melt Extrusion (HME) would result in formulations have advantageous properties.

The API+Soluplus+SLS polymer matrix and API+Soluplus+Poloxamer 407 demonstrated the best redissolution from micro-evaporation studies, and thus these two formulations are prepared as SDD prototypes and evaluated further for amorphous nature and redissolution behavior in the next Example.

### Example 3

### Spray Dried Dispersion Formulations

### Preparation of PA-824 SDD Prototype Trials

Preparation of PA-824 Spray Dried Dispersion (SDD) prototypes was performed on the Buchi B-290 spray dryer and with lead polymer matrices identified from miscibility modeling and ASD matrix screening studies. The composition of SDD prototype formulations are listed below in Table 26. Spray solution of each formulation were prepared by first dissolving required amount of API in 100 mL of solvent, followed by surfactant and lastly polymer. Selection of the solvent for spray drying was based on solubility of the API, polymer and surfactant. Inlet and outlet temperature are some of the most critical process parameters for spray drying; inlet temperature increases outlet temperature proportionally. Further, outlet temperature is established based on evaporation temperature of the solvent (MeOH is 64.6° C.); outlet temperature is sufficiently high so that solvent evaporates off at a controlled rate but is not too high to cause any degradation of the formulation. Resulting SDD from each trial was characterized by XRD for determination of residual crystallinity or if API was retained in amorphous state.

TABLE 26
PA-824 SDD Prototype Trials

| SDD | | | Solid |
|---|---|---|---|
| Prototype | Formulation | Spray | Content |
| Trial | Composition | Solution | (% w/v) |
| SDD Trial | PA-824-Soluplus-SLS | DCM:MeOH | 5% |
| 1 | (30-60-10) | (1:1) | |
| SDD Trial | PA-824-Soluplus-Poloxamer | MeOH | 10% |
| 2 | 407 | | |
| | (30-60-10) | | |
| SDD Trial | PA-824-Soluplus-SLS | MeOH | 5% |
| 3 | (30-60-10) | | |
| SDD Trial | PA-824-Kollidon VA | MeOH | 5% |
| 4 | 64-Soluplus | | |
| | (30-35-35) | | |
| SDD Trial | PA-824-Soluplus-SLS | MeOH | 5% |
| 5 | (15-75-10) | | |
| SDD Trial | PA-824-Kollidon VA | MeOH | 5% |
| 6 | 64-Soluplus | | |
| | (10-63-27) | | |
| SDD Trial | PA-824-HPMCAS-M | MeOH | 5% |
| 7 | (10-90) | | |

### Preparation of SDD Prototypes for PK Study

Three PA-824 SDD formulations were selected for a PK study. The SDD prototypes were prepared on the Buchi B-290 spray dryer. The composition of SDD prototype formulations are listed below in Table 27. Spray solution of each formulation were prepared by first dissolving required amount of API in 400 mL of solvent, followed by surfactant and lastly polymer. The bulk SDD material was placed in a vacuum oven for drying at 35° C. for 24 hours. Each SDD prototype was characterized by XRD and DSC for determination of residual crystallinity or if API was retained in amorphous state. Residual solvent testing was also performed on each SDD prototype after 24 hours of vacuum oven drying.

TABLE 27
PA-824 SDD for PK Study

| SDD | | | Solid |
|---|---|---|---|
| Prototype | | Spray | Content |
| Code | Formulation Composition | Solution | (% w/v) |
| SDD 1 | PA-824-Soluplus-SLS (15-75-10) | MeOH | 5% |
| SDD 2 | PA-824-Kollidon VA 64-Soluplus | MeOH | 5% |
| | (10-63-27) | | |
| SDD 3 | PA-824-HPMCAS-M (10-90) | MeOH | 5% |

Capsule Fill with PA-824 Crushed Tablets for PK Study

The PA-824 crushed tablet fill into capsule with 30 mg strength was assigned the formulation ID/product name: PA-824 Crushed Tablets in Capsule. PA-824 200 mg tablets (total tablet weight 800 mg) were grinded into powder using mortar and pestle and passed through 18 mesh sieves. For a strength of 30 mg PA-824 per capsule, 120 mg+/−1.5% of powder filled into each capsule. Capsule type was V Caps Plus, Size 0, White Opaque, Coni Snap. Six (6) capsules were selected at random for disintegration testing. From remaining capsules, 10 capsules were filled into 60cc HDPE bottle.

Capsule Fill with PA-824 SDD Prototypes for PK Study

Each SDD formulation was blended with Microcrystalline Cellulose (MCC); the formulation composition for each of the SDD filled into capsules is listed below in Tables 28-32. For each capsule fill batch, the required amount of PA-824 SDD was weighed into a 60cc HDPE bottle. The required amount of MCC was weighed and added to the bottle and then blended using turbula for 3 minutes.

### SDDI Fill Into Capsules, 30 mg Strength (Formulation ID PA-824 SDDI-C2)

The target batch size was 35 capsules total (9.1 grams total blend with of SDD1+MCC). For SDD1-C2 capsules with a strength of 30 mg PA-824 per capsule, 259.7 mg+/−10 mg powder blend was filled into each capsule. Capsule type was V Caps Plus, Size 0, White Opaque, Coni Snap. Six (6) capsules were selected at random for disintegration testing. From remaining capsules, 10 capsules were filled into 60cc HDPE bottle.

TABLE 28
SDD1 Fill in Capsules, 30 mg Strength

| | |
|---|---|
| Product Name: | PA-824 SDD1 - C2 |
| Strength | 30 mg of PA-824 per capsule |
| Capsule Type | V Caps Plus, Size 0, White Opaque, Coni Snap |
| Capsule Fill | PA-824 SDD1 (PA-824-Soluplus-SLS 15-75-10 Lot |
| | No.: 2020-159-054) + MCC. Fill with 259.7 mg +/− |
| | 10 mg powder blend into each capsule. |

PA-824 SDD1 - C2 Formulation Composition

| Material | Composition (%) | Weight (mg) |
|---|---|---|
| PA-824 API | 11.6% | 30.0 |
| Soluplus | 57.8% | 150.0 |
| Sodium Lauryl Sulfate | 7.7% | 20.0 |
| (SLS) | | |
| Microcrystalline Cellulose | 23.0% | 59.7 |
| (MCC) | | |
| Total | 100.0% | 259.7 |

### SDD2 Fill Into Capsules, 30 mg Strength (Formulation ID PA-824 SDD2-C3)

The target batch size was 30 capsules (11.7 grams total blend with SDD2+MCC). For SDD2 -C3 capsules with a strength of 30 mg PA-824 per capsule, 389.6 mg+/−15 mg powder blend was filled into each capsule. Capsule type was V Caps Plus, Size 00, Swedish Orange, Coni Snap. 6 capsules were selected at random for disintegration testing. From remaining capsules, 10 capsules were filled into 60cc HDPE bottle. Due to limitations in dosing monkeys with size 00, these capsules were not used for the monkey PK study.

TABLE 29
SDD2 Fill in Capsules, 30 mg Strength

| | |
|---|---|
| Product Name: | PA-824 SDD2 - C3 |
| Strength | 30 mg of PA-824 per capsule |
| Capsule Type | V Caps Plus, Size 00, Swedish Orange, Coni Snap |
| Capsule Fill | PA-824 SDD2 (PA-824-Kollidon VA64-Soluplus |
| | 10-63-27) + MCC. Fill with 389.6 mg +/− 15 mg powder |
| | blend into each capsule. |

PA-824 SDD2 - C3 Formulation Composition

| Material | Composition (%) | Weight (mg) |
|---|---|---|
| PA-824 API | 7.7% | 30.0 |
| Kollidon VA64 | 48.5% | 189.0 |
| Soluplus | 20.8% | 81.0 |
| Microcrystalline | 23.0% | 89.6 |
| Cellulose (MCC) | | |
| Total | 100.0% | 389.6 |

### SDD3 Fill Into Capsules, 30 mg Strength (Formulation ID: PA-824 SDD3-C4)

The target batch size was 30 capsules (11.7 grams total blend with SDD3+MCC). For SDD3 -C4 capsules with a strength of 30 mg PA-824 per capsule, 389.6 mg+/−15 mg powder blend was filled into each capsule. Capsule type was V Caps Plus, Size 00, Swedish Orange, Coni Snap. Six capsules were selected at random for disintegration testing. From remaining capsules, ten capsules were filled into 60cc HDPE bottle. Due to limitations with dosing monkeys with size 00 capsules, these capsules were not used for the monkey PK study.

TABLE 30
SDD3 Fill in Capsules, 30 mg Strength

| | |
|---|---|
| Product Name: | PA-824 SDD3 - C4 |
| Strength | 30 mg of PA-824 per capsule |
| Capsule Type | V Caps Plus, Size 00, Swedish Orange, Coni Snap |
| Capsule Fill | PA-824 SDD3 (PA-824-HPMCAS-M (10-90) Lot No.: |
| | 2020-159-058) + MCC. Fill with 389.6 mg +/− |
| | 15 mg powder blend into each capsule. |

PA-824 SDD3 - C4 Formulation Composition

| Material | Composition (%) | Weight (mg) |
|---|---|---|
| PA-824 API | 7.7% | 30.0 |
| HPMCAS-M | 69.3% | 270.0 |
| Microcrystalline Cellulose | 23.0% | 89.6 |
| (MCC) | | |
| Total | 100.0% | 389.6 |

### SDD2 Fill Into Capsules, 15 mg Strength (Formulation ID PA-824 SDD2 -C3)

The target batch size was 51 capsules (10.0 grams total blend with SDD2+MCC). For SDD2-C3 capsules with a strength of 15 mg PA-824 per capsule, 194.8 mg+/−10 mg powder blend was filled into each capsule. Capsule type was V Caps Plus, Size 0, White Opaque, Coni Snap 6 capsules were selected at random for disintegration testing. In order to achieve a total dose of 30 mg PA-824, two capsules were administered at dosing. From remaining capsules, 20 capsules were filled into 60cc HDPE bottle.

TABLE 31
SDD2 Fill in Capsules, 15 mg Strength

| | |
|---|---|
| Product Name: | PA-824 SDD2 - C3 |
| Strength | 15 mg of PA-824 per capsule |
| Capsule Type | V Caps Plus, Size 0, White Opaque, Coni Snap |
| Capsule Fill | PA-824 SDD2 (PA-824-Kollidon VA64-Soluplus |
| | 10-63-27 Lot No.: 2020-159-068) + MCC. Fill with |
| | 194.8 mg +/− 10 mg powder blend into each capsule. |

PA-824 SDD2 - C3 Formulation Composition

| Material | Composition (%) | Weight (mg) |
|---|---|---|
| PA-824 API | 7.7% | 15.0 |
| Kollidon VA64 | 48.5% | 94.5 |
| Soluplus | 20.8% | 40.5 |
| Microcrystalline Cellulose | 23.0% | 44.8 |
| (MCC) | | |
| Total | 100.0% | 194.8 |

### SDD3 Fill Into Capsules, 15 mg Strength (Formulation ID: PA-824 SDD3-C4)

The target batch size was 51 capsules (10.0 grams total blend with SDD3+MCC). For SDD3-C4 capsules with a strength of 15 mg PA-824 per capsule, 194.8 mg+/−10 mg powder blend was filled into each capsule. Capsule type was V Caps Plus, Size 0, White Opaque, Coni Snap 6 capsules were selected at random for disintegration testing. In order to achieve a total dose of 30 mg PA-824, two capsules were administered at dosing. From remaining capsules, 20 capsules were filled into 60 cc HDPE bottle.

TABLE 32
SDD3 Fill in Capsules, 15 mg Strength

| | |
|---|---|
| Product Name: | PA-824 SDD3 - C4 |
| Strength | 15 mg of PA-824 per capsule |
| Capsule Type | V Caps Plus, Size 0, White Opaque, Coni Snap |
| Capsule Fill | PA-824 SDD3 (PA-824-HPMCAS-M (10-90) Lot No.: |
| | 2020-159-070) + MCC. Fill with 194.8 mg +/− 10 mg |
| | powder blend into each capsule. |

PA-824 SDD3 - C4 Formulation Composition

| Material | Composition (%) | Weight (mg) |
|---|---|---|
| PA-824 API | 7.7% | 15.0 |
| HPMCAS-M | 69.3% | 135.0 |
| Microcrystalline Cellulose | 23.0% | 44.8 |
| (MCC) | | |
| Total | 100.0% | 194.8 |

### Preparation of Spray Dried Dispersion Prototypes

### Preparation of PA-824 SDD Prototypes Trials

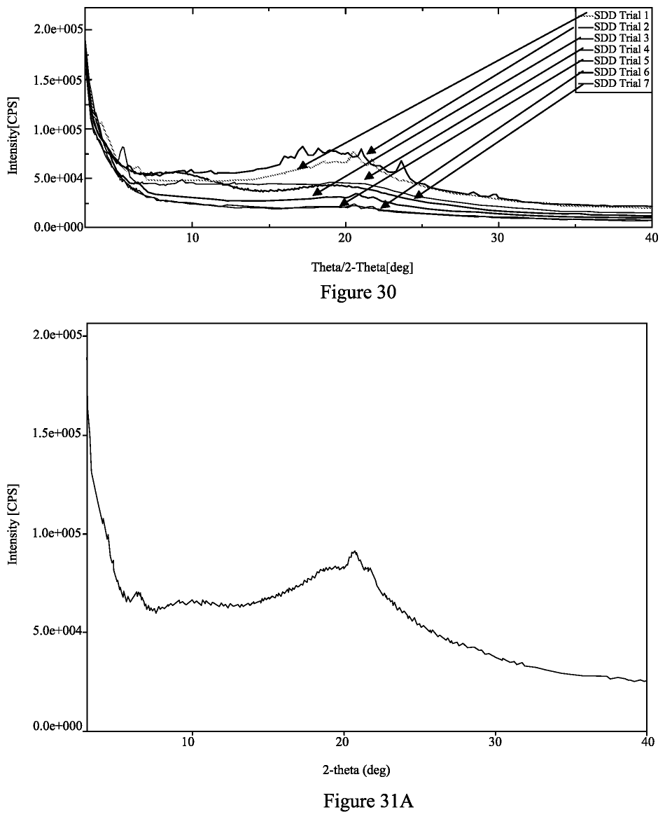

Table 33 below shows the SDD prototype trial data. FIG. 30 shows the XRD for each SDD trial and an overlay with the API. SDD Trial 1 formulation performed the best in micro-evaporation screening studies. This SDD is substantially amorphous, it did show some residual crystallinity. SDD Trial 2 formulation was the second best performing polymer matrix in micro-evaporation screening study but showed more residual crystallinity than SDD Trial 1. SDD Trial 1 formulation was evaluated further as SDD Trial 3 with a different solvent (MeOH) and reduced solid content (5%), however, it did show residual crystallinity. SDD Trial 4 formulation is a two-polymer matrix with Kollidon VA 64 as primary/stabilizer polymer and Soluplus as solubilizer, however, it did show residual crystallinity. SDD Trial 3 and Trial 4 were evaluated further with reduced drug load as Trial 5 and Trial 6, respectively. Both SDD Trial 5 and 6 show some residual crystallinity but not as significant as the respective trials with higher drug load. FIG. 30 shows the XRD overlay for SDD trials 1 through 7, in which the residual crystalline peaks seen correlate to the API crystalline peaks. SDD Trial 7 formulation was another lead polymer from micro-evaporation screening studies but with reduced drug load and shows no residual crystallinity, rather a halo indicating the API is retained in an amorphous state. SDD matrix formulations from Trial 5, 6 and 7 were selected for a PK study. Each of the formulations of SDD Trials 1-7 are considered to be at least substantially amorphous and show, or are contemplated to show, advantages compared to crystalline forms.

TABLE 33
PA-824 SDD Prototype Trials 1 through 7 Results

| SDD | | |
|---|---|---|
| Prototype | | |
| Trial | Formulation Composition | XRD Result |
| SDD Trial | PA-824-Soluplus-SLS (30-60-10) | Residual crystallinity |
| 1 | | |
| SDD Trial | PA-824-Soluplus-Poloxamer 407 | Residual crystallinity |
| 2 | (30-60-10) | |
| SDD Trial | PA-824-Soluplus-SLS (30-60-10) | Residual crystallinity |
| 3 | | |
| SDD Trial | PA-824-Kollidon VA 64-Soluplus | Residual crystallinity |
| 4 | (30-35-35) | |
| SDD Trial | PA-824-Soluplus-SLS (15-75-10) | Residual crystallinity |
| 5 | | |
| SDD Trial | PA-824-Kollidon VA 64-Soluplus | Residual crystallinity |
| 6 | (10-63-27) | |
| SDD Trial | PA-824-HPMCAS-M (10-90) | Amorphous |
| 7 | | |

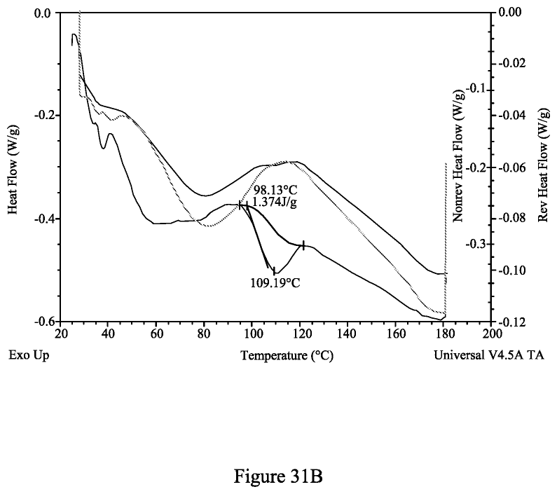

Table 34 below shows the SDD prototype code, composition, and residual solvent (after 24-hours vacuum oven drying) for SDD matrix formulations selected for filling into capsules and running in a monkey PK study. SDD 1 is a matrix from micro-evaporation screening studies and reduced drug load to prevent recrystallization. SDD 2 is a matrix with Kollidon VA 64 as the primary/stabilizer polymer and Soluplus acting as a solubilizer and reduced drug load to prevent recrystallization. SDD 3 is a matrix based on HSP from miscibility modeling and with reduced drug load to prevent recrystallization. A second batch of SDD2 and SDD3 was prepared to fill into size 0 capsules. FIGS. 31-35 show the XRD and DSC analysis results for each of the SDD prototypes. For SDD1, a wide endotherm is observed at 109° C. in the DSC thermogram and some crystalline peaks are seen in XRD analysis, which indicates some residual crystallinity. For SDD2 and SDD3, no API melting peak is observed in DSC analysis and an amorphous halo is seen in XRD analysis, which is indicative of amorphous nature for these prototypes.

TABLE 34
PA-824 SDD Prototypes for PK Study Results

| SDD | | Residual Solvent | | |
|---|---|---|---|---|
| Prototype | | MeOH | Tg from | |
| Code | Formulation Composition | (ppm) | DSC | |
| SDD 1 | PA-824-Soluplus-SLS | 273 | — | |
| | (15-75-10) | | | |
| SDD 2 | PA-824-Kollidon VA | 321 | 91.5° | C. |
| | 64-Soluplus (10-63-27) | | | |
| SDD 3 | PA-824-HPMCAS-M | 253 | 90.11° | C. |
| | (10-90) | | | |
| SDD 2 | PA-824-Kollidon VA | 0 | 89.25° | C. |
| | 64-Soluplus (10-63-27) | | | |
| SDD 3 | PA-824-HPMCAS-M | 0 | 92.35° | C. |
| | (10-90) | | | |

Capsule Fill with PA-824 Crushed Tablets for PK Study

Table 35 below shows resulting average fill weight and disintegration time for the PA-824 crushed tablet fill into capsule. This batch of capsules was included in a monkey PK study as the control arm.

TABLE 35
PA-824 Crushed Tablets Results

| Capsule Product | Capsule Fill | Disintegration |
|---|---|---|
| Name | Weight | Time |
| PA-824 Crushed | Min: 119.3 mg | 3:32 (min:sec) |
| Tablets in Capsule | Max: 121.8 mg | |
| | Mean: 120.7 mg | |
| | RSD: 0.5% | |

Capsule Fill with PA-824 SDD Prototypes for PK Study

Table 36 below shows the resulting average fill weight and disintegration time for each batch of SDD fill into capsules. These three batches of SDD fill in capsules were included in a monkey PK study.

TABLE 36
Capsule Fill Details and Disintegration Times

| Capsule | | PA-824 | | |
|---|---|---|---|---|
| Product | Capsule | Strength | Capsule Fill | Disintegration |
| Name | Size | per Capsule | Weight | Time |
| PA-824 | Size 0 | 30 mg | Min: 258.8 mg | 7:50 (min:sec) |
| SDD1 - C2 | | | Max: 263.3 mg | |
| | | | Mean: 260.4 mg | |
| | | | RSD: 0.5% | |
| PA-824 | Size 00 | 30 mg | Min: 387.4 mg | 7:14 (min:sec) |
| SDD2 - C3 | | | Max: 401.8 mg | |
| | | | Mean: 389.5 mg | |
| | | | RSD: 0.7% | |
| PA-824 | Size 00 | 30 mg | Min: 385.4 mg | 6:55 (min:sec) |
| SDD3 - C4 | | | Max: 397.0 mg | |
| | | | Mean: 388.1 mg | |
| | | | RSD: 0.54% | |
| PA-824 | Size 0 | 15 mg | Min: 192.2 mg | 6:43 (min:sec) |
| SDD2 - C3 | | | Max: 198.2 mg | |
| | | | Mean: 194.6 mg | |
| | | | RSD: 0.74% | |
| PA-824 | Size 0 | 15 mg | Min: 190.9 mg | 5:24 (min:sec) |
| SDD3 - C4 | | | Max: 196.9 mg | |
| | | | Mean: 194.2 mg | |
| | | | RSD: 0.55% | |

In summary, the addition of surfactant to the polymer matrix with Soluplus showed higher API concentration than with only Soluplus. However, when these two lead matrices were prepared as SDD trial 1 and SDD trial 2, residual crystallinity was observed. Reducing the drug load in SDD trials reduced the amount of residual crystallinity for the same formulations. The SDD trial 7 with HPMCAS-M showed to be amorphous. Residual crystallinity seen in the SDD formulations is likely to reduce bioavailability as the API is recrystallizing.

### Example 4

### Pharmacokinetic study of Pretomanid (PA-824) Following Single Oral Doses of Four Different Capsule Formulations in Non-Naïve Cynomolgus Monkeys

### Objective

The objective of this study was to evaluate and compare the pharmacokinetic properties of Pretomanid (PA-824) the four different capsule formulations formed in Example 3 in non-naïve cynomolgus monkeys following oral administration to four male non-naïve cynomolgus monkeys using a crossover design.

The pharmacokinetics of PA-824 was evaluated following oral administration of four different capsule formulations to four male cynomolgus monkeys. There was a seven-day washout period between the dosing of formulations.

PA-824 Crushed Tablet in Capsule (C1) formulation and PA-824 SDD1 in Capsule (C2) formulation consisting of 30 mg PA-824 per capsule were administered orally to male monkeys as a single dose of 30 mg PA-824 per monkey. PA-824 SDD2 in Capsule (C3) formulation and PA-824 SDD3 in Capsule (C4) formulation consisting of 15 mg PA-824 per capsule were administered orally to male monkeys as a single dose of 30 mg PA-824 per monkey. Further details of these capsules may be found in the previous examples. Capsules were administered to fasted monkeys and flushed with 5 mL/kg water. There were a 7-day washout period between doses.

Blood samples were collected from the cephalic vein into K₃EDTA tubes for processing to plasma and drug concentration assessment at the following timepoints: predose and 0.25-, 0.5-, 1-, 2-, 4-, 8-, and 24-hours post PA-824 administration. Plasma concentrations of PA-824 were determined using LC-MS/MS with a limitation of quantitation (LLOQ) of 1.00 ng/mL. The pharmacokinetic parameters were determined by non-compartmental analysis using WinNonlin 8.0.

### Results and Discussion

For PA-824 Crushed Tablet in Capsule(C1) and PA-824 SDD1 in Capsule (C2), the monkeys were dosed 1 capsule/monkey (30 mg/monkey). For PA-824 SDD2 in Capsule (C3) and PA-824 SDD3 in Capsule (C4), the monkeys were dosed 2 capsules/monkey (30 mg/monkey). The PK parameters were calculated based on the nominal dose level of 30 mg/monkey. Following oral administrations of PA-824 in capsule formulations PA-824 Crushed Tablet in Capsule(C1), PA-824 SDD1 in Capsule (C2), PA-824 SDD2 in Capsule (C3), and PA-824 SDD3 in Capsule (C4) in male monkeys, the AUC₀₋ₜ of PA-824 were 9739, 11461, 11295, and 13425 hr*ng/ml respectively, the corresponding AUC₀₋ₜ were 9892, 11675, 11448, and 13661 hr*ng/ml. The Cₘₐₓ in capsule formulations PA-824 Crushed Tablet in Capsule(C1), PA-824 SDD1 in Capsule (C2), PA-824 SDD2 in Capsule (C3), and PA-824 SDD3 in Capsule (C4) in male monkeys were 1063, 1118, 1225, and 1618 ng/mL, respectively, occurring at 3.25, 4.50, 3.00, and 3.00 hr, with the half-lives of 3.53, 3.70, 3.72 and 3.55 hr, respectively.

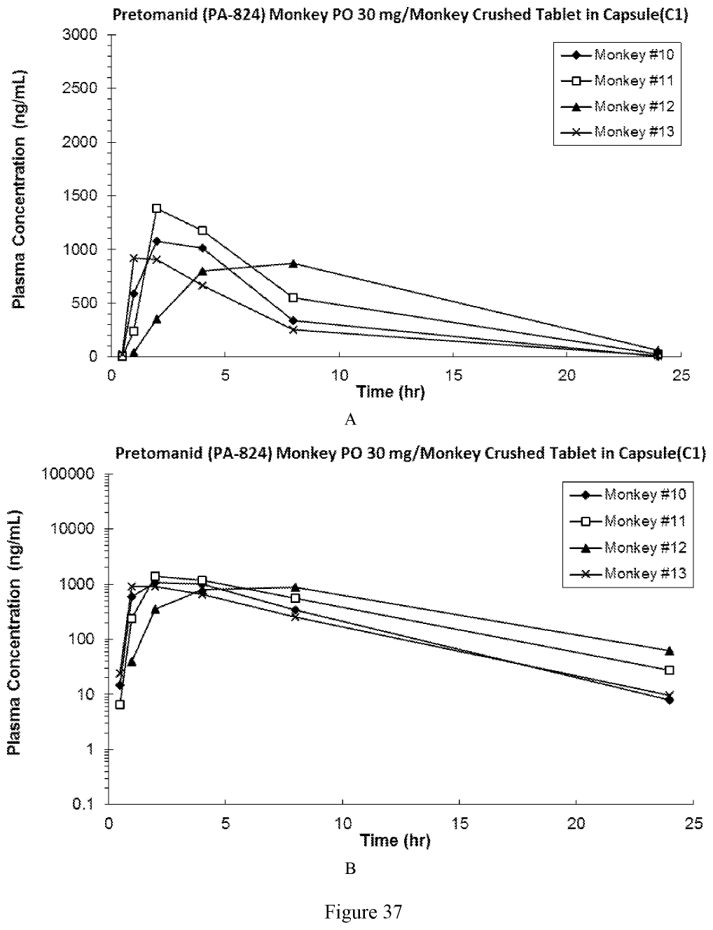

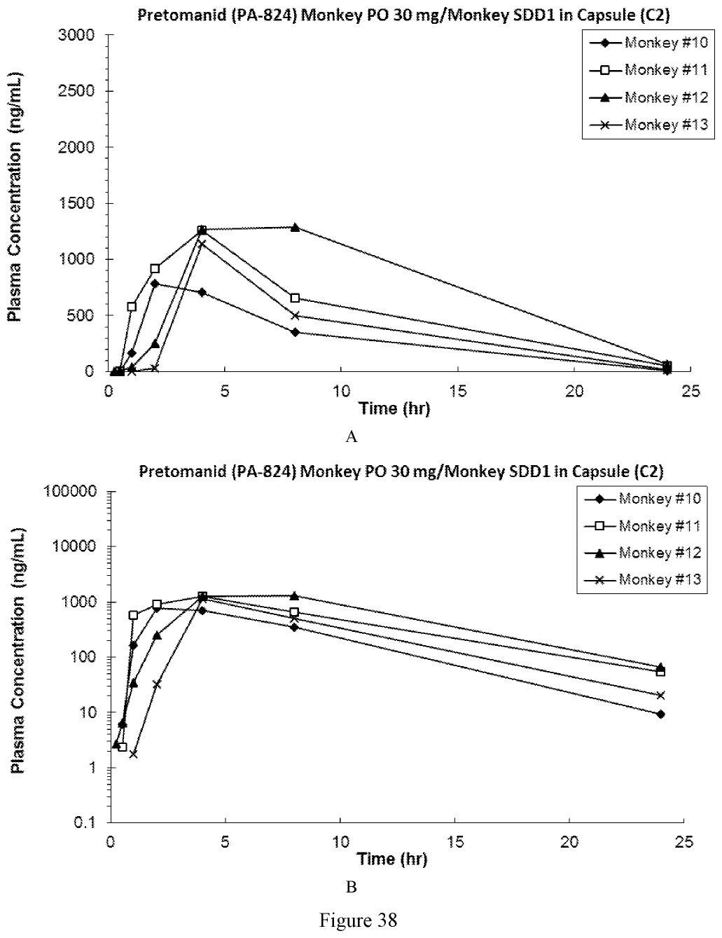

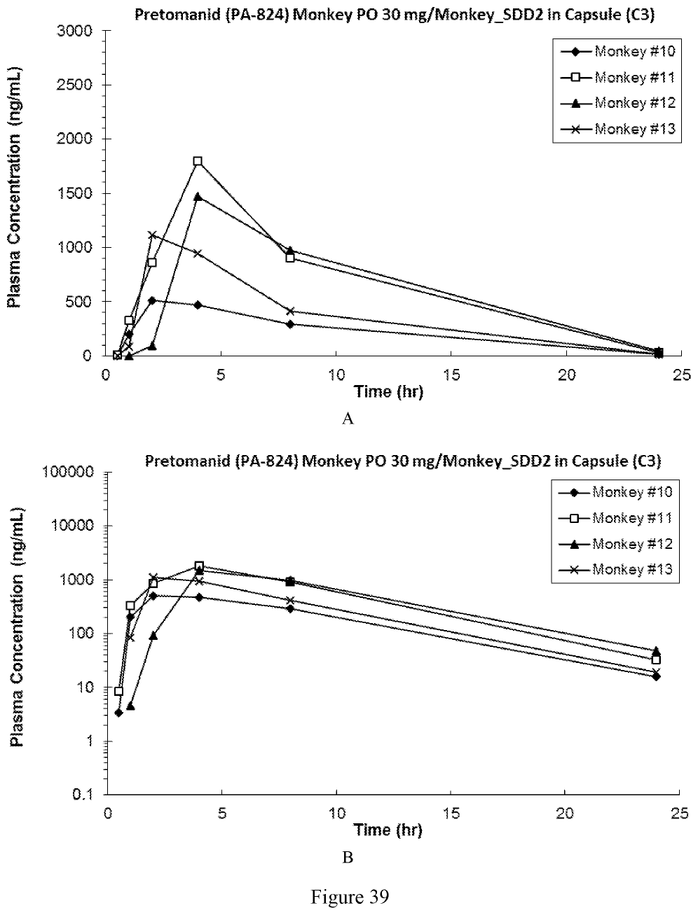

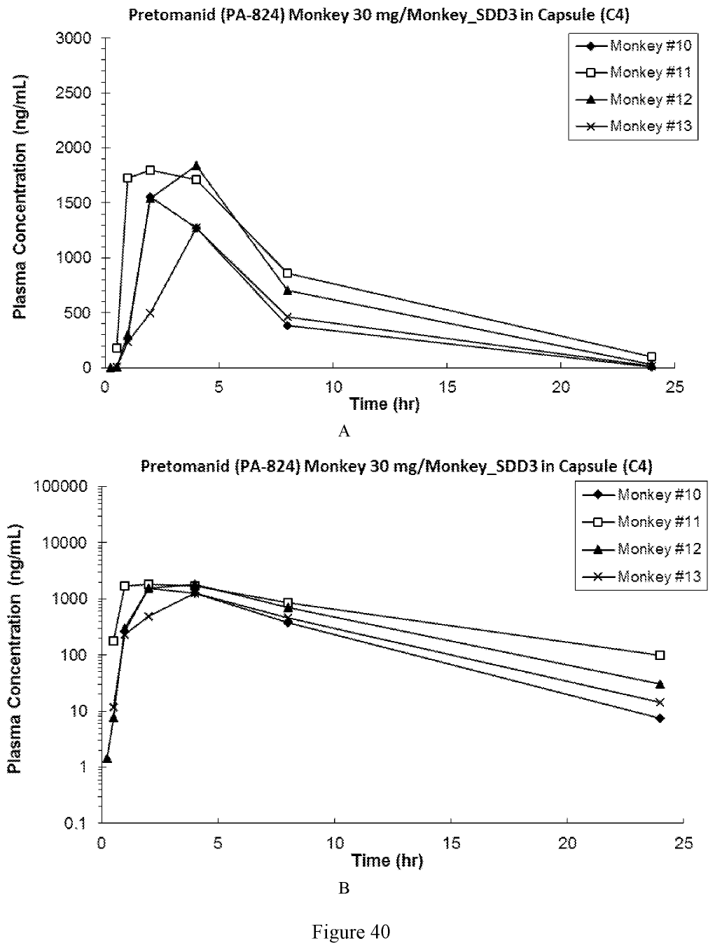

The individual plasma concentrations for PA-824 following oral administration are listed in Table 37 (PA-824 Crushed Tablet in Capsule(C1)), Table 38 (PA-824 SDD1 in Capsule (C2)), Table 39 (PA-824 SDD2 in Capsule (C3)), and Table 40 (PA-824 SDD3 in Capsule (C4)). The corresponding plasma concentration versus time curves are shown in FIG. 37 (PA-824 Crushed Tablet in Capsule(C1)), FIG. 38 (PA-824 SDD1 in Capsule (C2)), FIG. 39 (PA-824 SDD2 in Capsule (C3)), and FIG. 40 (PA-824 SDD3 in Capsule (C4)) for PA-824 following oral administration. The pharmacokinetic parameters for individual animals following oral administration of PA-824 are summarized in Table 41(PA-824 Crushed Tablet in Capsule(C1)), Table 42 (PA-824 SDD1 in Capsule (C2)), Table 43 (PA-824 SDD2 in Capsule (C3)), Table 44 (PA-824 SDD3 in Capsule (C4)). The mean±SD PK parameters and the corresponding plasma concentrations versus time curves are shown in Table 45.

TABLE 37
Individual and Mean Plasma Concentration of PA-824 Following Oral Administration of
the Crushed Tablet in Capsule (C1) Formulation at 30 mg/monkey to Male Monkeys
Pretomanid (PA-824) PO 30 mg/Monkey_Crushed Tablet in Capsule (C1)

| Time | Calculated Concentration (ng/mL) | | | | | | |
|---|---|---|---|---|---|---|---|
| (h) | Monkey #10 | Monkey #11 | Monkey #12 | Monkey #13 | Mean | SD | CV % |
| Predose | BLOQ | BLOQ | BLOQ | BLOQ | NA | NA | NA |
| 0.25 | BLOQ | BLOQ | BLOQ | BLOQ | NA | NA | NA |
| 0.5 | 14.9 | 6.61 | BLOQ | 24.2 | 11.4 | 10.5 | 91.6 |
| 1 | 586 | 236 | 38.9 | 923 | 446 | 3905 | 87.5 |
| 2 | 1080 | 1380 | 356 | 909 | 931 | 430 | 46.2 |
| 4 | 1010 | 1180 | 799 | 662 | 913 | 229 | 25.0 |
| 8 | 339 | 553 | 869 | 257 | 505 | 273 | 54.1 |
| 24 | 7.98 | 27.8 | 63.0 | 9.56 | 27.1 | 25.6 | 94.4 |

BLOQ—below limit of quantitation (1.00 ng/mL) and zero was used for mean calculation;
NA: Not applicable

TABLE 38
Individual and Mean Plasma Concentration of PA-824 Following Oral Administration
of the SDD1 in Capsule (C2) at 30 mg/monkey to Male Monkeys
Pretomanid (PA-824) PO 30 mg/Monkey_SDD1 in Capsule (C2)

| Time | Calculated Concentration (ng/mL) | | | | | | |
|---|---|---|---|---|---|---|---|
| (h) | Monkey #10 | Monkey #11 | Monkey #12 | Monkey #13 | Mean | SD | CV % |
| Predose | BLOQ | BLOQ | BLOQ | BLOQ | NA | NA | NA |
| 0.25 | BLOQ | BLOQ | 2.68 | BLOQ | 0.67 | NA | NA |
| 0.5 | 6.06 | 2.36 | 6.60 | BLOQ | 3.76 | 3.13 | 83.4 |
| 1 | 166 | 579 | 35.0 | 1.77 | 195 | 265 | 136 |
| 2 | 782 | 919 | 252 | 32.1 | 496 | 422 | 85.1 |
| 4 | 705 | 1260 | 1270 | 1140 | 1094 | 266 | 24.3 |
| 8 | 351 | 654 | 1290 | 499 | 699 | 413 | 59.2 |
| 24 | 9.29 | 53.9 | 67.3 | 20.6 | 37.8 | 27.3 | 72.3 |

BLOQ—below limit of quantitation (1.00 ng/mL) and zero was used for mean calculation.
NA: Not applicable

TABLE 39
Individual and Mean Plasma Concentration of PA-824 Following Oral Administration
of the SDD2 in Capsule (C3) at 30 mg/monkey to Male Monkeys
Pretomanid (PA-824) PO 30 mg/Monkey_SDD2 in Capsule (C3)

| | Calculated Concentration (ng/mL) | | | | | | |
|---|---|---|---|---|---|---|---|
| Time (h) | Monkey #10 | Monkey #11 | Monkey #12 | Monkey #13 | Mean | SD | CV % |
| Predose | BLOQ | BLOQ | BLOQ | BLOQ | NA | NA | NA |
| 0.25 | BLOQ | BLOQ | BLOQ | BLOQ | NA | NA | NA |
| 0.5 | 3.36 | 8.56 | BLOQ | BLOQ | 2.98 | NA | NA |
| 1 | 202 | 328 | 4.57 | 84.0 | 155 | 141 | 91.3 |
| 2 | 511 | 864 | 91.7 | 1120 | 647 | 446 | 69.0 |
| 4 | 469 | 1800 | 1470 | 948 | 1172 | 585 | 49.9 |
| 8 | 290 | 903 | 975 | 415 | 646 | 344 | 53.2 |
| 24 | 15.6 | 32.2 | 47.5 | 19.1 | 28.6 | 14.5 | 50.6 |

BLOQ—below limit of quantitation (1.00 ng/mL) and zero was used for mean calculation;
NA: Not applicable

TABLE 40
Individual and Mean Plasma Concentration of PA-824 Following Oral Administration
of the SDD3 in Capsule (C4) at 30 mg/monkey to Male Monkeys
Pretomanid (PA-824) PO 30 mg/Monkey_SDD3 in Capsule (C4)

| | Calculated Concentration (ng/mL) | | | | | | |
|---|---|---|---|---|---|---|---|
| Time (h) | Monkey #10 | Monkey #11 | Monkey #12 | Monkey #13 | Mean | SD | CV % |
| Predose | BLOQ | BLOQ | BLOQ | BLOQ | NA | NA | NA |
| 0.25 | BLOQ | BLOQ | 1.44 | BLOQ | 0.36 | NA | NA |
| 0.5 | BLOQ | 176 | 7.65 | 12.0 | 48.9 | 84.9 | 174 |
| 1 | 262 | 1730 | 302 | 233 | 632 | 733 | 116 |
| 2 | 1560 | 1800 | 1540 | 500 | 1350 | 579 | 42.9 |
| 4 | 1270 | 1710 | 1840 | 1270 | 1523 | 296 | 19.5 |
| 8 | 384 | 858 | 701 | 464 | 602 | 217 | 36.1 |
| 24 | 7.44 | 98.5 | 30.2 | 14.3 | 37.6 | 41.7 | 111 |

BLOQ—below limit of quantitation (1.00 ng/mL) and zero was used for mean calculation
NA: Not applicable

TABLE 41
Non-Compartmental Pharmacokinetic Parameters of PA-824 Following Oral Administration
Crushed Tablet in Capsule (C1) at 30 mg/monkey to Male Monkeys

| Parameters | Monkey #10 | Monkey #11 | Monkey #12 | Monkey #13 | Mean | SD |
|---|---|---|---|---|---|---|
| Actual Dose | 8.11 | 7.14 | 6.98 | 6.98 | 7.30 | 0.54 |
| (mg/kg) | | | | | | |
| T_{1/2} (hr) | 2.89 | 3.70 | 4.23 | 3.30 | 3.53 | 0.57 |
| Tₘₐₓ (hr) | 2.00 | 2.00 | 8.00 | 1.00 | 3.25 | 3.20 |
| Cₘₐₓ (ng/mL) | 1080 | 1380 | 869 | 923 | 1063 | 230 |
| Cₗₐₛₜ (ng/mL) | 7.98 | 27.8 | 63.0 | 9.56 | 27.1 | 25.6 |
| AUCₗₐₛₜ | 8551 | 11543 | 12164 | 6700 | 9739 | 2568 |
| (hr*ng/mL) | | | | | | |
| AUC_{0-∞} | 8584 | 11691 | 12548 | 6746 | 9892 | 2702 |
| (hr*ng/mL) | | | | | | |
| AUC_{Extr} (%) | 0.388 | 1.27 | 3.06 | 0.674 | 1.35 | 1.20 |
| CL/F (mL/min/kg) | 15.7 | 10.2 | 9.27 | 17.2 | 13.1 | 4.0 |
| MRT_{0-∞} (hr) | 5.29 | 6.19 | 8.28 | 5.20 | 6.24 | 1.43 |
| AUC_{0-∞}/D | 1058 | 1637 | 1798 | 966 | 1365 | 414 |
| (hr*kg*ng/mL/mg) | | | | | | |
| Tₗₐₛₜ (hr) | 24.0 | 24.0 | 24.0 | 24.0 | 24.0 | 0.0 |

TABLE 42
Non-Compartmental Pharmacokinetic Parameters of PA-824 Following Oral
Administration SDD1 in Capsule (C2) at 30 mg/monkey to Male Monkeys

| Parameters | Monkey #10 | Monkey #11 | Monkey #12 | Monkey #13 | Mean | SD |
|---|---|---|---|---|---|---|
| Actual Dose | 7.89 | 7.14 | 6.82 | 6.98 | 7.21 | 0.48 |
| (mg/kg) | | | | | | |
| T_{1/2} (hr) | 3.16 | 4.41 | 3.76 | 3.46 | 3.70 | 0.54 |
| Tₘₐₓ (hr) | 2.00 | 4.00 | 8.00 | 4.00 | 4.50 | 2.52 |
| Cₘₐₓ (ng/mL) | 782 | 1260 | 1290 | 1140 | 1118 | 233 |
| Cₗₐₛₜ (ng/mL) | 9.29 | 53.9 | 67.3 | 20.6 | 37.8 | 27.3 |
| AUCₗₐₛₜ | 7000 | 12565 | 17656 | 8625 | 11461 | 4745 |
| (hr*ng/mL) | | | | | | |
| AUC_{0-∞} | 7042 | 12908 | 18020 | 8728 | 11675 | 4897 |
| (hr*ng/mL) | | | | | | |
| AUC_{Extr} (%) | 0.601 | 2.66 | 2.02 | 1.18 | 1.62 | 0.91 |
| CL/F (mL/min/kg) | 18.7 | 9.22 | 6.31 | 13.3 | 11.9 | 5.4 |
| MRT_{0-∞} (hr) | 5.96 | 7.08 | 7.93 | 6.95 | 6.98 | 0.80 |
| AUC_{0-∞}/D | 893 | 1808 | 2642 | 1250 | 1648 | 762 |
| (hr*kg*ng/mL/mg) | | | | | | |
| Tₗₐₛₜ (hr) | 24.0 | 24.0 | 24.0 | 24.0 | 24.0 | 0.0 |

TABLE 43
Non-Compartmental Pharmacokinetic Parameters of PA-824 Following Oral
Administration SDD2 in Capsule (C3) at 30 mg/monkey to Male Monkeys

| Parameters | Monkey #10 | Monkey #11 | Monkey #12 | Monkey #13 | Mean | SD |
|---|---|---|---|---|---|---|
| Actual Dose | 7.89 | 7.50 | 6.82 | 6.98 | 7.30 | 0.49 |
| (mg/kg) | | | | | | |
| T_{1/2} (hr) | 3.99 | 3.41 | 3.93 | 3.57 | 3.72 | 0.28 |
| Tₘₐₓ (hr) | 2.00 | 4.00 | 4.00 | 2.00 | 3.00 | 1.15 |
| Cₘₐₓ (ng/mL) | 511 | 1800 | 1470 | 1120 | 1225 | 551 |
| Cₗₐₛₜ (ng/mL) | 15.6 | 32.2 | 47.5 | 19.1 | 28.6 | 14.5 |
| AUCₗₐₛₜ | 5351 | 16234 | 14682 | 8911 | 11295 | 5062 |
| (hr*ng/mL) | | | | | | |
| AUC_{0-∞} | 5441 | 16392 | 14951 | 9009 | 11448 | 5123 |
| (hr*ng/mL) | | | | | | |
| AUC_{Extr} (%) | 1.65 | 0.967 | 1.80 | 1.09 | 1.38 | 0.41 |
| CL/F (mL/min/kg) | 24.2 | 7.63 | 7.60 | 12.9 | 13.1 | 7.8 |
| MRT_{0-∞} (hr) | 6.65 | 6.55 | 7.56 | 6.06 | 6.70 | 0.63 |
| AUC_{0-∞}/D | 690 | 2186 | 2192 | 1291 | 1590 | 734 |
| (hr*kg*ng/mL/mg) | | | | | | |
| Tₗₐₛₜ (hr) | 24.0 | 24.0 | 24.0 | 24.0 | 24.0 | 0.0 |

TABLE 44
Non-Compartmental Pharmacokinetic Parameters of PA-824 Following Oral
Administration SDD3 in Capsule (C4) at 30 mg/monkey to Male Monkeys

| Parameters | Monkey #10 | Monkey #11 | Monkey #12 | Monkey #13 | Mean | SD |
|---|---|---|---|---|---|---|
| Actual Dose | 7.89 | 7.50 | 6.82 | 6.98 | 7.30 | 0.49 |
| (mg/kg) | | | | | | |
| T_{1/2} (hr) | 2.73 | 4.93 | 3.42 | 3.12 | 3.55 | 0.96 |
| Tₘₐₓ (hr) | 2.00 | 2.00 | 4.00 | 4.00 | 3.00 | 1.15 |
| Cₘₐₓ (ng/mL) | 1560 | 1800 | 1840 | 1270 | 1618 | 263 |
| Cₗₐₛₜ (ng/mL) | 7.44 | 98.5 | 30.2 | 14.3 | 37.6 | 41.7 |
| AUCₗₐₛₜ | 10312 | 18584 | 15311 | 9495 | 13425 | 4294 |
| (hr*ng/mL) | | | | | | |
| AUC_{0-∞} | 10341 | 19284 | 15460 | 9559 | 13661 | 4572 |
| (hr*ng/mL) | | | | | | |
| AUC_{Extr} (%) | 0.283 | 3.63 | 0.963 | 0.673 | 1.39 | 1.52 |
| CL/F (mL/min/kg) | 12.7 | 6.48 | 7.35 | 12.2 | 9.68 | 3.22 |
| MRT_{0-∞} (hr) | 5.14 | 7.08 | 6.02 | 6.13 | 6.09 | 0.80 |
| AUC_{0-∞}/D | 1311 | 2571 | 2267 | 1370 | 1880 | 636 |
| (hr*kg*ng/mL/mg) | | | | | | |
| Tₗₐₛₜ (hr) | 24.0 | 24.0 | 24.0 | 24.0 | 24.0 | 0.0 |

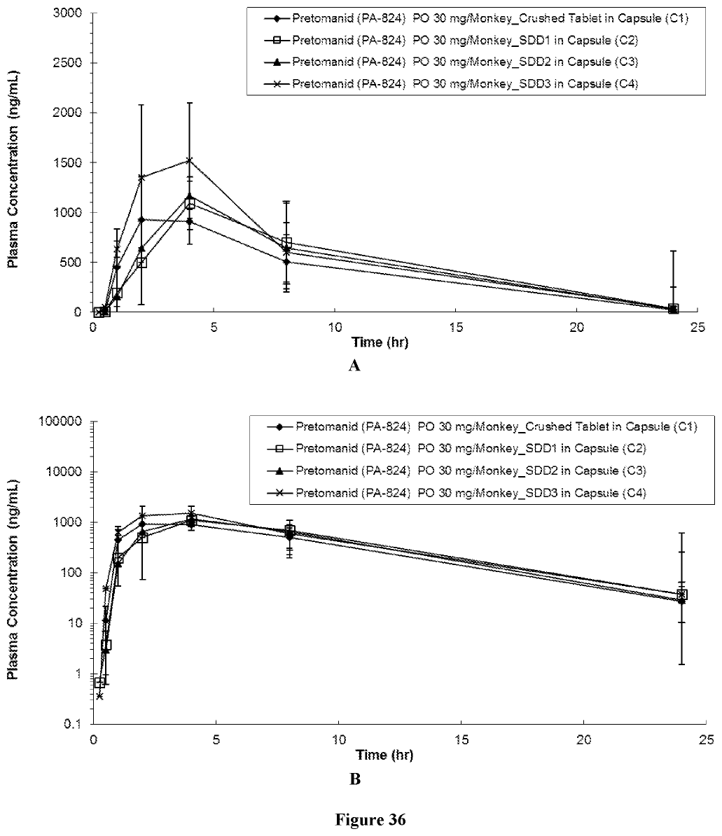

FIG. 36 also provides results for this experiment.

In summary, following oral administration of PA-824 in four different capsule formulations, specifically, PA-824 Crushed Tablet in Capsule(C1), PA-824 SDD1 in Capsule (C2), PA-824 SDD2 in Capsule (C3), and PA-824 SDD3 in Capsule (C4), to four male non-naive cynomolgus monkeys using a crossover design, the AUC₀₋ₜ of PA-824 was 9739, 11461, 11295, and 13425 hr*ng/mL, respectively. Tₘₐₓ was 3.25, 4.50, 3.00, and 3.00 hr for PA-824 capsules of PA-824 Crushed Tablet in Capsule(C1), PA-824 SDD1 in Capsule (C2), PA-824 SDD2 in Capsule (C3), and PA-824 SDD3 in Capsule (C4), respectively. The four different capsule formulations had similar half-lives for PA-824 were observed following oral administration of the 4 kinds of capsule formulations in male monkeys ranging from 3.53 to 3.72 hr.

TABLE 45
Mean (±SD) Non-Compartmental Pharmacokinetic Parameters of PA-824
in Male Monkeys (n = 4) Following Oral Administration

| | PA-824 | | | |
|---|---|---|---|---|
| | Crushed | PA-824 | PA-824 | PA-824 |
| | Tablet in | SDD1 in | SDD2 in | SDD3 in |
| Formulation | Capsule | Capsule | Capsule | Capsule |
| ID | (C1) | (C2) | (C3) | (C4) |
| Dose | 30 | 30 | 30 | 30 |
| (mg/monkey) | | | | |
| Tₘₐₓ (hr) | 3.25 ± 3.20 | 4.50 ± 2.52 | 3.00 ± 1.15 | 3.00 ± 1.15 |
| Cₘₐₓ | 1063 ± 230 | 1118 ± 233 | 1225 ± 551 | 1618 ± 263 |
| (ng/mL) | | | | |
| Tₗₐₛₜ (hr) | 24 | 24 | 24 | 24 |
| AUC₀₋ₜ | 9739 ± 2568 | 11461 ± 4745 | 11295 ± 5062 | 13425 ± 4294 |
| (hr*ng/mL) | | | | |
| AUC_{0-∞} | 9892 ± 2702 | 11675 ± 4897 | 11448 ± 5123 | 13661 ± 4572 |
| (hr*ng/mL) | | | | |
| AUC_{% ext} (%) | 1.35 ± 1.20 | 1.62 ± 0.91 | 1.38 ± 0.41 | 1.39 ± 1.52 |
| T_{1/2} (hr) | 3.53 ± 0.57 | 3.70 ± 0.54 | 3.72 ± 0.28 | 3.55 ± 0.96 |
| MRT_{0-∞} (hr) | 6.24 ± 1.43 | 6.98 ± 0.80 | 6.70 ± 0.63 | 6.09 ± 0.80 |
| CL/F | 13.1 ± 4.0 | 11.9 ± 5.4 | 13.1 ± 7.8 | 9.68 ± 3.22 |
| (mL/min/kg) | | | | |

### Methods

### Materials for Capsules

PA-824 Crushed Tablet in Capsule(C1), PA-824 SDD1 in Capsule (C2), PA-824 SDD2 in Capsule (C3), and PA-824 SDD3 in Capsule (C4).

### Materials for Standard

PA-824 drug substance powder was used.

### Animals

The in-life part of this study was conducted at Suzhou Xishan Zhongke Laboratories. In this study, total four male cynomolgus monkeys (body weight: 3.7-4.4 kg) were used. Individual animal body weights and dosing dates are listed in Table 46. The diet was provided throughout the in-life portion of the study with the exception of overnight fasting prior to dosing through 4 hrs post dose. Drinking water was available daily ad libitum to all animals.

### Study Design

This study was a crossover design. All four animals received PA-824 in four different capsule formulations at 30 mg/monkey.

TABLE 46
PK Study-Design Details

| | | Body weight | |
|---|---|---|---|
| Formulation | Animal ID | (kg) | Dosage |
| PA-824 Crushed | Monkey 10 | 3.7 | 1 capsule |
| Tablet in Capsule(C1) | Monkey 11 | 4.2 | 1 capsule |
| (30 mg/capsule) | Monkey 12 | 4.3 | 1 capsule |
| | Monkey 13 | 4.3 | 1 capsule |
| PA-824 SDD1 in | Monkey 10 | 3.8 | 1 capsule |
| Capsule (C2) | Monkey 11 | 4.2 | 1 capsule |
| (30 mg/capsule) | Monkey 12 | 4.4 | 1 capsule |
| | Monkey 13 | 4.3 | 1 capsule |
| PA-824 SDD2 in | Monkey 10 | 3.8 | 2 capsules |
| Capsule (C3) | Monkey 11 | 4.0 | 2 capsules |
| (15 mg/capsule) | Monkey 12 | 4.4 | 2 capsules |
| | Monkey 13 | 4.3 | 2 capsules |
| PA-824 SDD3 in | Monkey 10 | 3.8 | 2 capsules |
| Capsule (C4) | Monkey 11 | 4.0 | 2 capsules |
| (15 mg/capsule) | Monkey 12 | 4.4 | 2 capsules |
| | Monkey 13 | 4.3 | 2 capsules |

Each monkey was orally administered one capsule or two capsules of PA-824 according to Table 46. Capsules were administered to fasted monkeys and flushed with 5 mL/kg water. There was a 7-day washout period between each dose.

### Sample Collection

Blood samples (approximately 1 mL) were collected via cephalic vein into K3EDTA tubes at pre-dose and 0.25-, 0.5-, 1-, 2-, 4-, 8-, and 24-hours post PA-824 administration. The blood samples were placed on ice and centrifuged at 3000 rpm for 10 minutes on 4° C. within 30 min of collection, then plasma samples were transferred to a tube and stored at −80° C. prior to analysis by LC-MS/MS.

### Assay

### Sample Preparation

An aliquot of 50 μL plasma sample was mixed with 200 μL methanol/acetonitrile (1:1, v/v) containing internal standard (Terfenadine: 5 ng/mL). The sample was vortexed and centrifuged for 15 mins, then a 50 μL of supernatant was transferred and diluted 5× with methanol/water (1:1, v/v, with 0.1% FA) for LC-MS/MS analysis.

### LC-MS MS Analysis

All separations were performed on a Kinetex 2.6μ C18 100A column (50 mm*3.00 mm) at 40° C. with a flow rate of 0.5 mL/min. Mobile phase A was 0.05% formic acid with 5 mM ammonium acetate in water and mobile phase B was 0.1% formic acid in acetonitrile. Chromatography used a linear gradient by maintaining 5% mobile phase B for 0.4 minute, 5 to 95% mobile phase B over 1.6 minute, followed by a 95% mobile phase B wash for 0.4 minute, fall to 5% mobile phase B within 0.01 min and a re-equilibration for 0.59 minute. Total run time was 3 minutes. The injection volume was 2 μL.

The mass spectrometer (API-6500, Applied Biosystems/MDS SCIEX Instruments, Foster City, CA) was operated in positive ion multiple reaction monitoring mode (MRM). Mass transition was 360.23/175.10 for PA-824 and 472.40/436.40 for Terfenadine (IS). The retention time for PA-824 and Terfenadine (IS) were 1.98 min and 1.92 min, respectively.

### Data Analysis

Standards and quality control (QC) samples were prepared in blank monkey plasma. The standard curve ranges were 1.00-2000 ng/mL with a lower limit of quantitation (LLOQ) of 1.00 ng/ml (three QC samples were used at 2, 500, 1600 ng/mL, with dilution quality control of 8000 ng/mL). For a batch with more than 10 samples, two sets of standard curve and QCs were included. If a batch contained 10 or fewer samples, only one standard curve and two sets of QCs were included. For each analytical batch, more than 75% of calculated standard curve values did not deviate by more than 20% of the nominal concentrations using a 1/x² weighted linear regression based on the ratio of analyte to internal standard peak areas, and more than two-thirds of the QC values were within 20% of the nominal concentrations and 50% QC values in one concentration should within 20% of the nominal concentration. For plasma concentrations below the lower limit of quantitation of 1.00 ng/mL, zero was used for mean calculations.

### Pharmacokinetic Analysis

The pharmacokinetic parameters of PA-824 were determined by non-compartmental analysis using WinNonlin Version 8.0 (Pharsight, Mountain View, CA). The area under the curve from the time of dosing to the last measurable concentration, AUC₀₋ₜ was calculated by the linear trapezoidal rule. The area under the concentration-time curve extrapolated to infinity, AUC_{0-∞}, was calculated as follows:

AUC-_{0-∞}=AUC₀₋ₜ+Cₗₐₛₜ/k

Where Cₗₐₛₜ is the last measurable concentration and k is the first order rate constant associated with the terminal elimination phase, estimated by linear regression of log concentration versus time.

AUC_{% ext}=Cₗₐₛₜ/k/AUC_{0-∞}*100%

The half-life (T_{1/2}) of the terminal elimination phase was estimated based on the following equation:

T_{1/2}=0.693/k

Additional parameters were calculated as follows:

CL/F=Dose/AUC_{0-∞}

Where CL is the clearance of PA-824 in L/hr/kg, Dose is the administered dose in mg/kg. Mean residence time (MRT) was calculated as follows:

MRT_{0-∞}=AUMC_{0-∞}/AUC_{0-∞}

Where area under the first moment curve extrapolated to infinity (AUMC_{0-∞}) was calculated as follows:

AUMC_{0-∞}=AUMCₗₐₛₜ+tₗₐₛₜ*Cₗₐₛₜ/k+Cₗₐₛₜ/k²

Nominal time was used for all pharmacokinetic calculations since there was no significant delay for sample collection reported.

### Example 5

### Enhancing Dissolution and Preventing Crystallization

### Crystallization Inhibition of PA-824 in Presence of HPMCAS

Induction time of crystallization was determined in the presence of three grades of HPMCAS: LF, MF and HF, in phosphate buffered saline (PBS), pH 6.5. Drug concentration studied was 80 μg/mL and polymer concentrations were 100 μg/mL and 10 μg/mL. Based on the results, the selected HPMCAS grade was further tested for crystallization inhibition in PBS pH 6.5 and in fasted simulated intestinal fluid (FaSSIF V1) and with polymer concentration of 1 mg/mL. Crystallization behavior in the two stages of dissolution was observed with PLM images and by recording the SEM and XRPD patterns.

### Preparation of PA-824 ASDs for Dissolution Study

PA-824 ASDs were prepared using solvent evaporation using a rotary evaporator. Solvent used was 1:1 dichloromethane:methanol with drug loading of 10-25% with different polymer compositions as described below, and at a total solid content of 10% w/v. Polymers used included HPMCAS-HF (referred to as PH), HPMCAS-HF with HPMCAS-MF (referred to as PMH), for comparison to example 3. In addition, HPMCAS-HF salts were also tested by adding base to form polymer salts. Factors studied were polymer type, drug loading, additives (surfactants) and polymer salts. Some example formulations are presented in Tables 47 and 48. Additional formulations studied are presented in the figures are based on these formulations based on varying the stated factors such as but not limited to drug loading and combining additives with polymer salts.

TABLE 47
Example formulations of PA-824 ASDs with HPMCAS
polymers at 10% Drug Loading and with additives

| | PA-824 | HPMCAS- | HPMCAS- | HPMCAS- | SLS | TPGS | Triethylamine |
|---|---|---|---|---|---|---|---|
| Sample ID | (mg) | LF (mg) | MF (mg) | HF (mg) | (mg) | (mg) | (mg) |
| PH-10 | 200 | | | 1800 | | | |
| PM-10 | 200 | | 1800 | | | | |
| PMH-10 | 200 | | 900 | 900 | | | |
| PLH-10 | 200 | 900 | 900 | | | | |
| PHS-10 | 200 | | | 1780 | 20 | | |
| PHTP-10 | 200 | | | 1780 | | 20 | |
| (HF-TPGS) | | | | | | | |
| PHTE-10 | 200 | | | 1664 | | | 133 |
| | | | | | | | (1:1 polymer |
| | | | | | | | molar ratio) |

TABLE 48
Example formulations of PA-824 ASDs with
HPMCAS-HF salts at 20% Drug Loading

| | | MW | | | |
|---|---|---|---|---|---|
| | pKa of | (g/mol) | PA-824 | HPMCAS-HF | Base |
| Sample ID | base | of base | (mg) | (mg) | (mg) |
| HF only (HF-20) | — | — | 400 | 1600.0 | — |
| HF-Triethylamine | 10.67 | 101.19 | 400 | 1481.3 | 118.7 |
| HF-DMEA | 9.32 | 89.14 | 400 | 1494.5 | 105.5 |
| (dimethylamino- | | | | | |
| ethanol) | | | | | |
| HF-MDEA (N- | 8.54 | 119.16 | 400 | 1462.0 | 138.0 |
| methyl- | | | | | |
| diethanolamine) | | | | | |
| HF-triethanolamine | 7.73 | 149.18 | 400 | 1430.9 | 169.1 |
| HF-Tris | 8.1 | 121.14 | 400 | 1459.9 | 140.1 |
| HF-Meglumine | 9.58 | 195.21 | 400 | 1385.7 | 214.3 |
| HF-Ammediol | 8.8 | 105.14 | 400 | 1477.0 | 123.0 |

The dissolution profiles of these ASDs were determined in two different conditions at a PA-824 concentration of 200 μg/mL (or 10 mg/50 mL): single stage test in 50 mL of PBS pH 6.5 for 1 hour or two stage pH-shift experiment with dissolution in 45 mL of hydrochloric acid (HCl) pH 1.6 for 1 hour followed by adjusting the pH to 6.5 by adding 5 mL of concentrated buffer. USP dissolution apparatus II at 150 RPM and 37° C. was used. Drug dissolution was measured in situ using fiber optic UV spectroscopy.

Dissolution was also determined in biorelevant media: single stage in FaSSIF V1 and two stage pH-shift experiment with FaSSGF for 1 hour followed by adjusting pH to FaSSIF V1.

### Results and Discussion

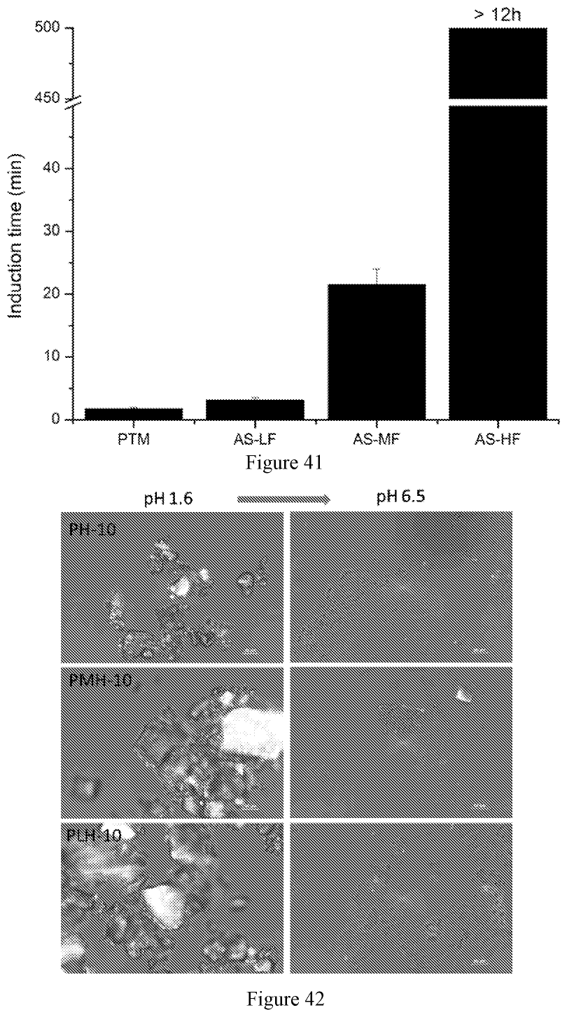

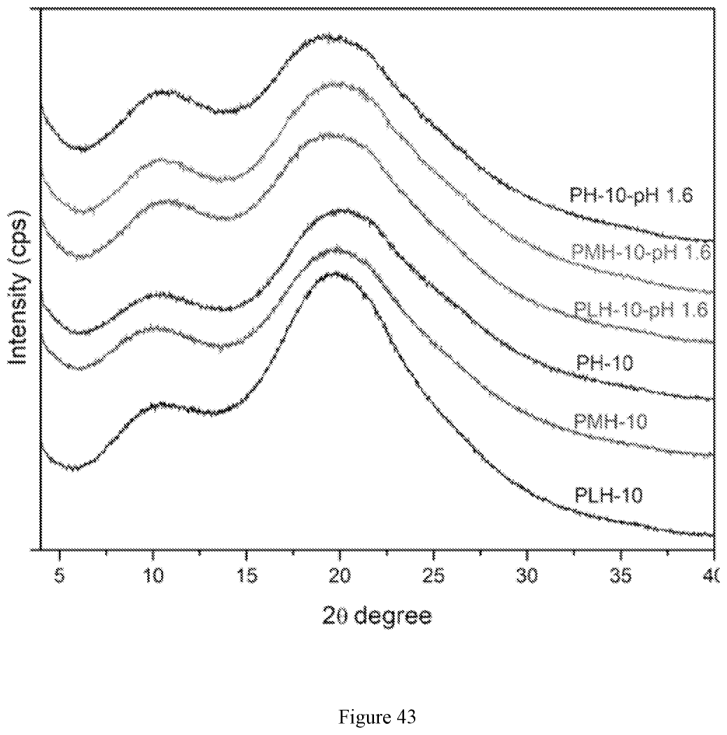

As presented in FIG. 41, the crystallization effect of HPMCAS grades was LF<MF<<HF, with HF grade showing significantly more inhibition of crystallization of PA-824 at polymer concentration of 100 μg/mL. At polymer concentration of 10 μg/mL of HPMCAS-HF, the induction time of PA-824 was about 77.5 minutes in PBS pH 6.5. At drug concentration of 160 μg/mL, the induction times were significantly higher in the presence of HPMCAS-HF concentration of 1 mg/mL (Table 49). FIG. 42 with the PLM images confirm that amorphous nature of PA-824 in the presence of HPMCAS grades at 10% drug loading in HCI pH 1.6 at 1 hour, and crystallization was observed after 1 hour at pH 6.5. FIG. 43 show the XRPD pattern of PA-824 confirming the amorphous nature at 1 hour in pH 1.6 in presence of all HPMCAS polymer grades tested.

TABLE 49
Induction time of crystallization of 160 ug/mL
PA-824 in presence of 1 mg/mL of HPMCAS-HF

| Medium | Induction Time (min) |
|---|---|
| PBS pH 6.5 | 1.39 ± 0.18 |
| FaSSIF V1 | 0.89 ± 0.26 |
| PBS pH 6.5 + HF (1 mg/mL) | 43.75 ± 1.34 |
| FaSSIF V1 + HF (1 mg/mL) | 77.6 ± 13.6 |

Based on the results (Table 49 and FIGS. 42 and 43), it was concluded that PA-824 has a relatively high amorphous solubility of about 160 μg/mL in PBS and about 300 g/mL in FaSSIF V1. PA-824 does have a high tendency to crystallize in aqueous media, and HPMCAS-HF prevents the crystallization.

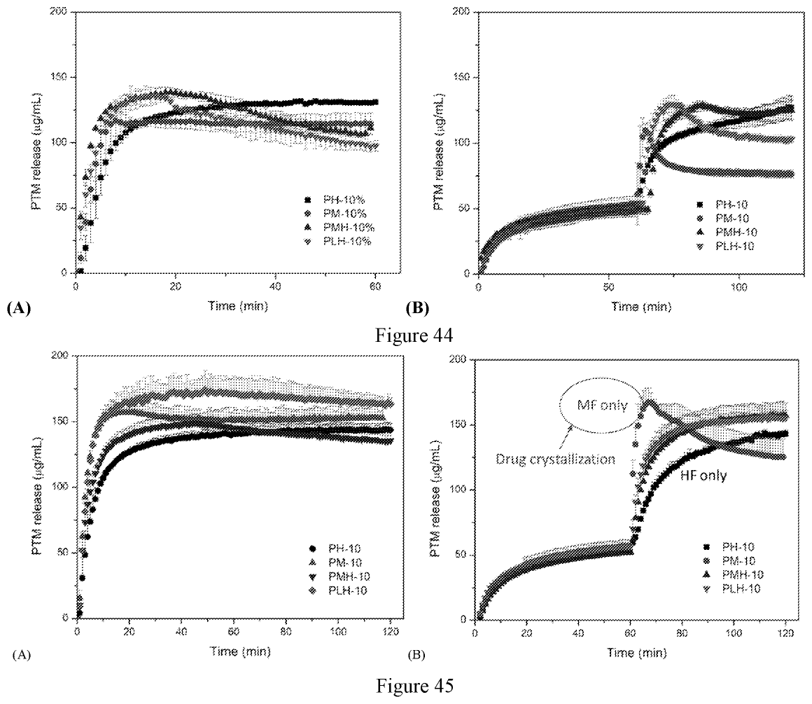

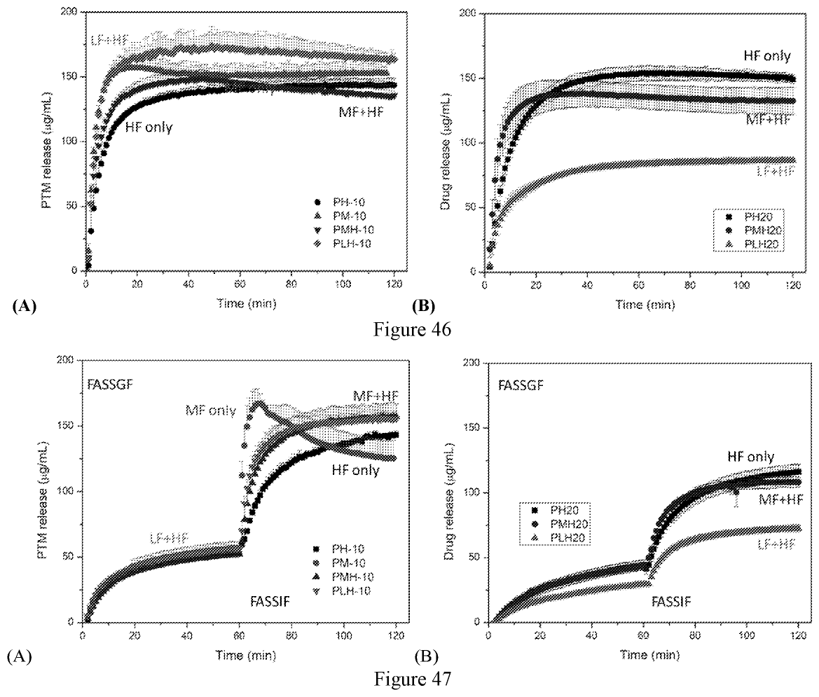

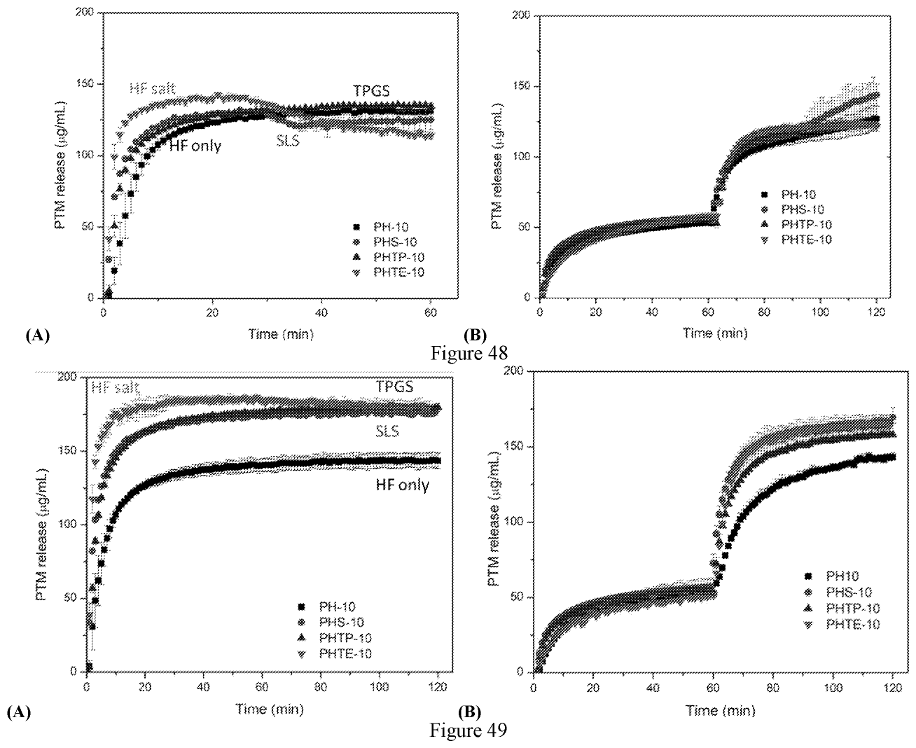

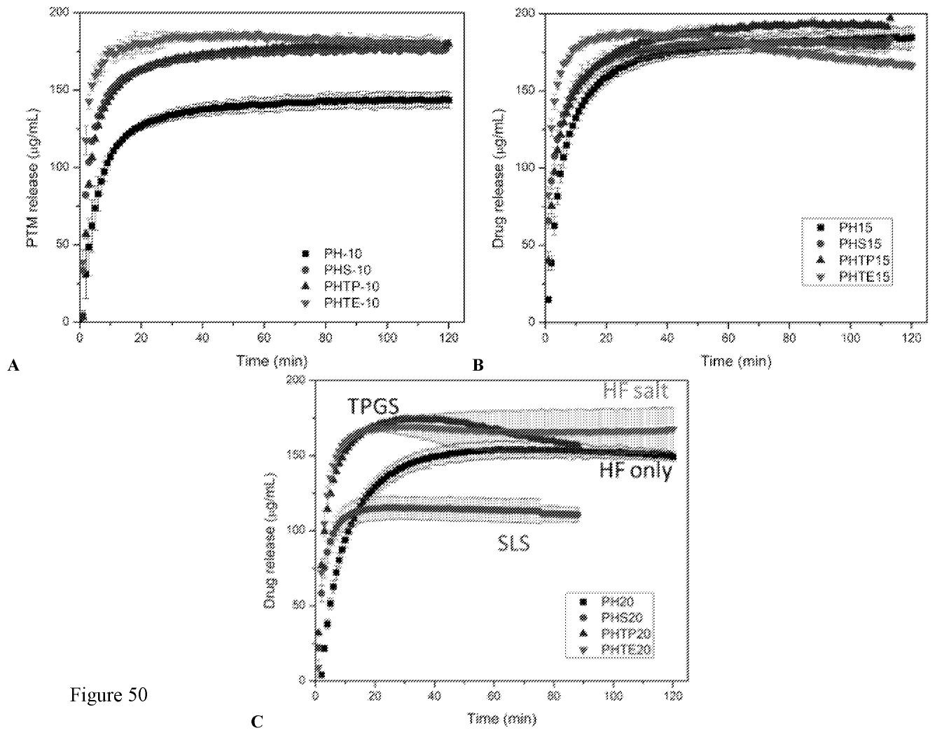

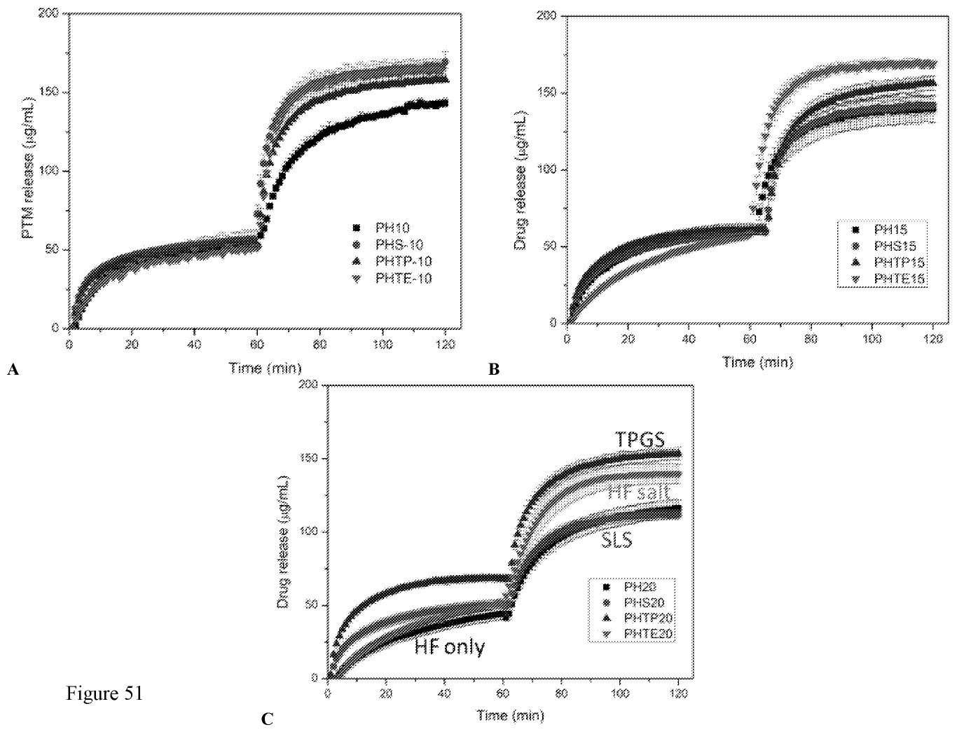

Dissolution profiles of the formulations studied are presented in FIGS. 44-47 for HPMCAS polymer grades and drug loadings. Dissolution profiles of the formulations studied are presented in FIGS. 48-51 for HPMCAS-HF polymer with additives (surfactants) and drug loadings.

Based on the results of the drug loading and polymer grades, it was shown that HPMCAS-HF plays an important role in inhibiting crystallization of PA-824 during dissolution. Combined polymers can maintain good drug release at low drug loading (10%) but not at high drug loading (20%). Based on the results of the effect of additives (surfactants), it was concluded that TPGS can be used to improve drug release.

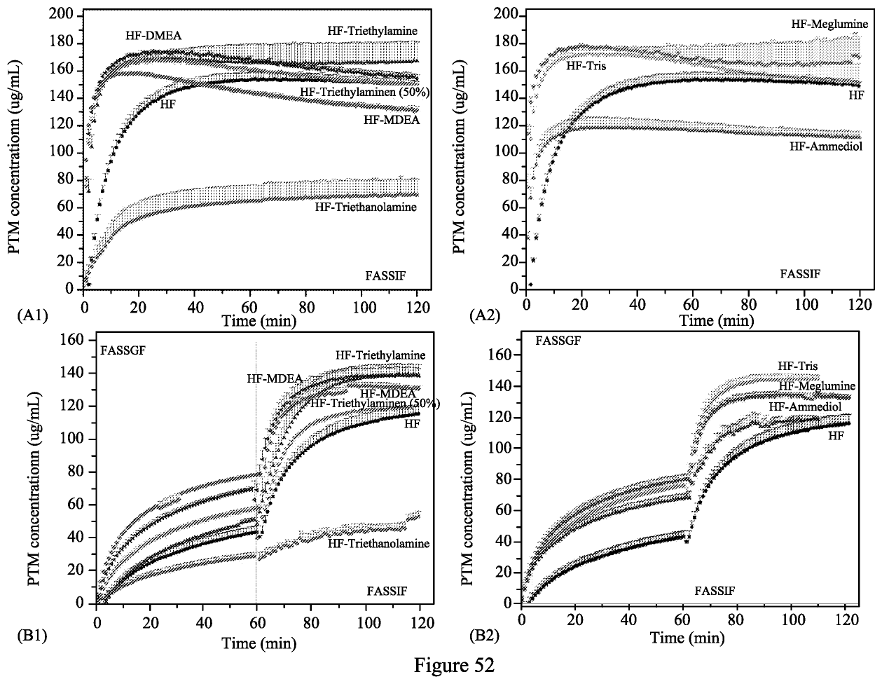

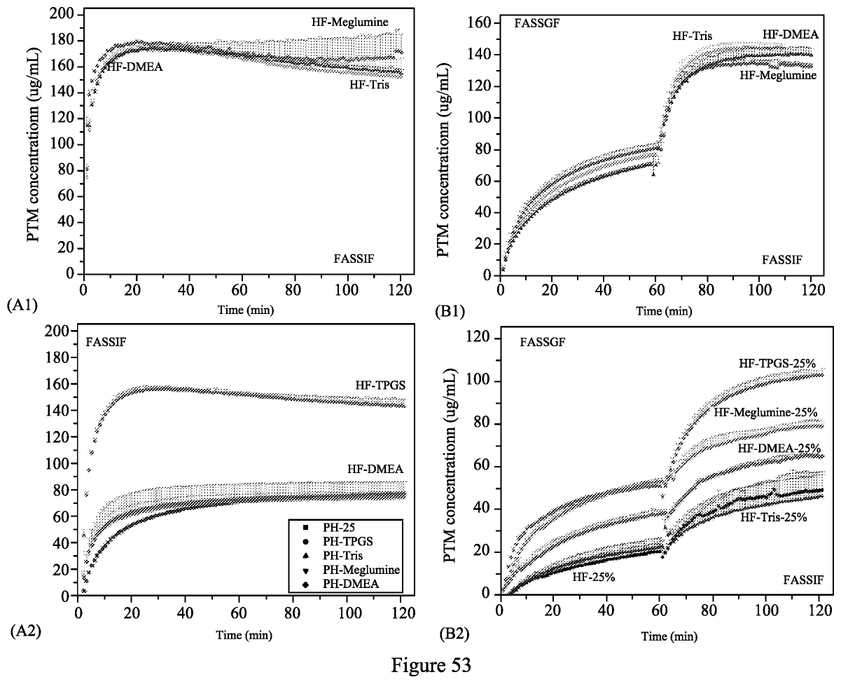

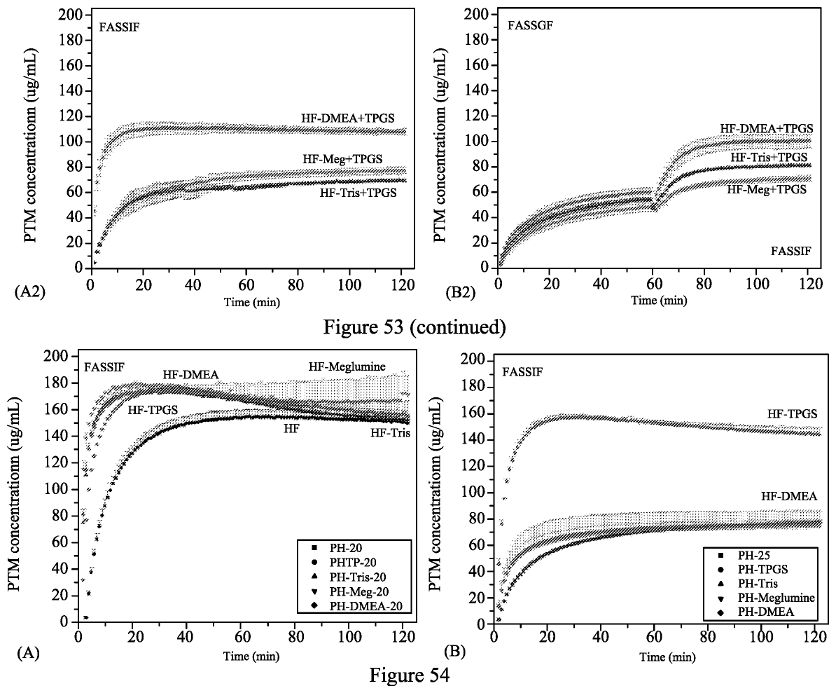

FIGS. 52-54 display the dissolution profiles of HPMCAS-HF as salts and with additives. The most promising salts were found to be HF-Meglumine, HF-DMEA and HF-Tris to improve drug release; with additives, HP-TPGS was also found to be promising. In general, the most promising salts at 20% drug loading resulted in higher drug release compared to 25% drug loading; PA-824 release from HF-TPGS at 20 and 25% drug loadings was not significantly different. Combining the promising HF-salts with TPGS (HF-salt-TPGS) at 25% drug loading did not have any advantage as it slowed the drug release compared to HF salt or HF-TPGS separately.

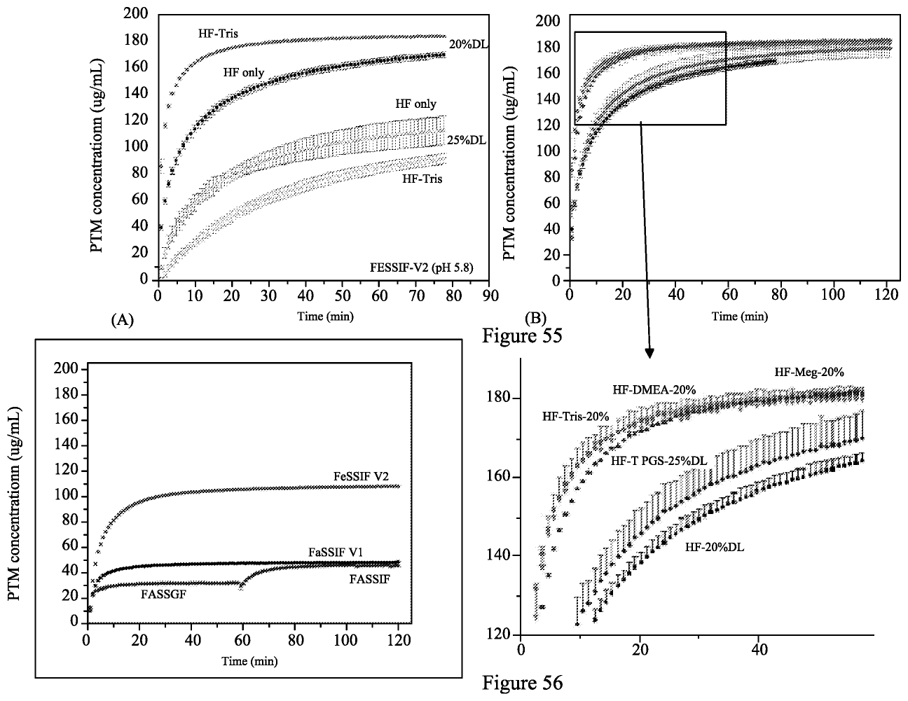

In the fed stage, the dissolution of PA-824 from these ASDs may change due to the impact of food. Therefore, dissolution was also tested in simulated fed medium (FeSSIF). The pH of fed stimulated fluids is about 5.0-5.8, which is below the dissolution pH threshold of polymer of 6.8 as reported by the HPMCAS-HF manufacturer. HPMCAS-HF has a pKa of about 5.15 and the polymer starts swelling and forming a colloidal solution at a pH around 5.7. With the polymer salt, a more basic microenvironment may be induced and enable higher release of polymer and drug. In addition, the dissolution of drug may be improved due to the increase of drug solubility in fed stimulated media. FIG. 55 shows the release profiles of PA-824 from the promising HF-salts and HF-TPGS in FeSSIF medium pH 5.8. At 20% drug loading, HF-Tris released about 90% of PA-824 in 1 hour compared to about 80% release with HF only. At 25% drug loading, drug release reduced significantly to about 40% from HF-Tris at 1 hour compared to about 50% from HF only. With HF-TPGS at 25% drug loading, about 90% PA-824 release was observed. Dissolution profiles of reference 200 mg PA-824 tablets (FIG. 56) show about 20% in FaSSIF at 1 hour and about 50% in FeSSIF, confirming the significant improvement in release with all formulations studied, and especially with the promising HF-salts and HF-TPGS.

Based on these results, it was concluded that HPMCAS-HF salt enhances drug release. ASD of PA-824 with HF-salts at 20% drug loading enhances drug release. Drug release is significantly reduced at higher drug loading of 25%. PA-824 has quite high amorphous solubility, especially in presence of surfactant or SIF.

Experiments are conducted on PA-824 ASDs at 20% drug loading with HF only, HF-Tris and HF-TPGS. In these experiments, HF is control sample, HF-Tris salt is used because it is a smaller counterion compared to the other promising HF-salts, and HF-TPGS is used because the additive formulation as it increased the drug release to a greater extent than HF-SLS.

### Example 6

### Formulation of PA-824 ASDs Into Capsules and Tablets

### Preparation and Characterization of Spray Dried ASD Tablet Formulations

Selected ASDs from example 5 were formulated into capsules and tablets. ASD processing was tested using spray drying and rotary evaporation (rotovap). In general, spray dried samples have a smaller size which may generate a faster release than rotovap sample. However, spray dried powders are more sensitive to moisture and may have poor flow. The ASDs by spray drying and rotovap were formulated into tablets and capsules and dissolution was compared against the formulation in powder form. The formulation composition was 150 mg of ASD powder at 20% drug loading equivalent to 30 mg PA-824, 30 mg of sodium starch glycolate (SSG), 30 mg of croscarmellose sodium, and 90 mg of microcrystalline cellulose (MCC) for a total target fill weight of 300 mg. HPMC capsules of size 0 were filled with the formulation equivalent to 30 mg of PA-824. Tablets of 300 mg target weight were compressed using a round 0.4375″ (11 mm) diameter tooling, with thickness of 4.3-4.5 mm. In order to improve the dissolution from capsules, addition of lubricants, magnesium stearate and colloidal silica were tried.

Dissolution of the tablet formulations was studied in FaSSIF, in FeSSIF, and FaSSGF to FaSSIF, and compared to the crushed reference PA-824 tablets filled as 30 mg strength in size 0 capsules. In order to obtain complete release of the drug, dissolution was also performed in PBS pH 6.5 with 0.5% surfactant, cetyltrimethylammonium bromide (CTAB).

Final selected tablet formulations were prepared with spray dried ASDs of HPMCAS-HF only, HPMCAS-HF-Tris salt and HPMCAS-HF-TPGS. ASDs were prepared at a 20% drug loading by solvent evaporation using a Buchi B-290 spray dryer. The composition for ASDs is noted in Table 50. PA-824, polymer (HPMCAS-HF) and counter ion (Tris base) or vitamin E tocopheryl polyethylene glycol succinate (TPGS) (if applicable) were dissolved in mixture of dichloromethane-methanol (1:1) at 10% w/v solid content. Base was added at a 1:1 molar ratio to polymer. Then ASDs were spray dried at 80° C., 95% aspirator and feed rate of 6 mL/min, followed by storage in a vacuum oven overnight to remove residual solvent.

To prepare tablets, the ASD powder was mixed with other excipients using a mortar and pestle using the tablet formulation described above. Tablets were prepared by direct compression using a single die and punch, size 0.4375 size die. Compression force was 500 pound-force (pf) or 0.22 ton-force (tf). Tablets have a diameter of 11.1 mm, and thickness of 4.3 mm. Average tablet hardness (n=6) was 2.2±0.5 kp, 1.7±0.2 kp, 1.3±0.3 kp for ASD of HF, HF-Tris, and HF-TPGS respectively. The average tablet weight (n=10) was 302.8±3.1 mg; 302.9±1.6 mg; and 300.5±1.6 mg for ASDs of HF, HF-Tris, and HF-TPGS respectively. Tablets were packaged and stored in 2 oz HDPE bottle with desiccant at ambient room temperature after preparation.

TABLE 50
ASD composition

| | | PA-824 | HPMCAS- | Tris base | TPGS | Solvent |
|---|---|---|---|---|---|---|
| ASDs | Batch No. | (mg) | HF (mg) | (mg) | (mg) | (mL) |
| HF (20%) | PA-824-HF-SD-002 | 2000.0 | 8000.0 | | | 100 |
| HF-Tris | PA-824-HF-Tris-SD-002 | 2000.0 | 7299.5 | 700.5 | | 100 |
| (20%) | | | | | | |
| HF-TPGS | PA-824-HF-TPGS-SD-002 | 2000.0 | 8000 | | 400 | 100 |
| (20%) | | | | | | |

All dissolution studies of PA-824 samples were conducted in triplicate in single stage or two stage pH-shift condition using a Hanson Vision G2 Classic 6 dissolution system (Teledyne Hanson Research, Chatsworth, CA). The tablet containing the ASD was added to 150 mL FASSIF V1, pH 6.5 and monitored for 1 h at 37° C., with 150 rpm of stirring. For pH-shift experiments, the tablet was first tested in 135 mL FASSGF, pH 1.6, followed by adding 15 mL of concentrated FASSIF buffer (pH 7.3) to achieve 150 mL FASSIF, pH 6.5 for 1 h. An in situ Rainbow fiber optic ultraviolet spectrometer with a 10 mm fiber optics (Pion, Billerica, MA, USA) was used to monitor drug concentration over time. Second derivative analysis was applied to correct the spectral baseline and a calibration curve of area under curve (AUC) of the range 390-410 nm was used to calculate the drug concentration. Maximum concentration of theoretically complete release was 200 μg/mL.

Short term stability of spray dried ASD powders was studied after storage for 1-4 months at room temperature, without desiccant. The stored samples were tested for dissolution. Degradation in the samples was tested by NMR.

### Results and Discussion

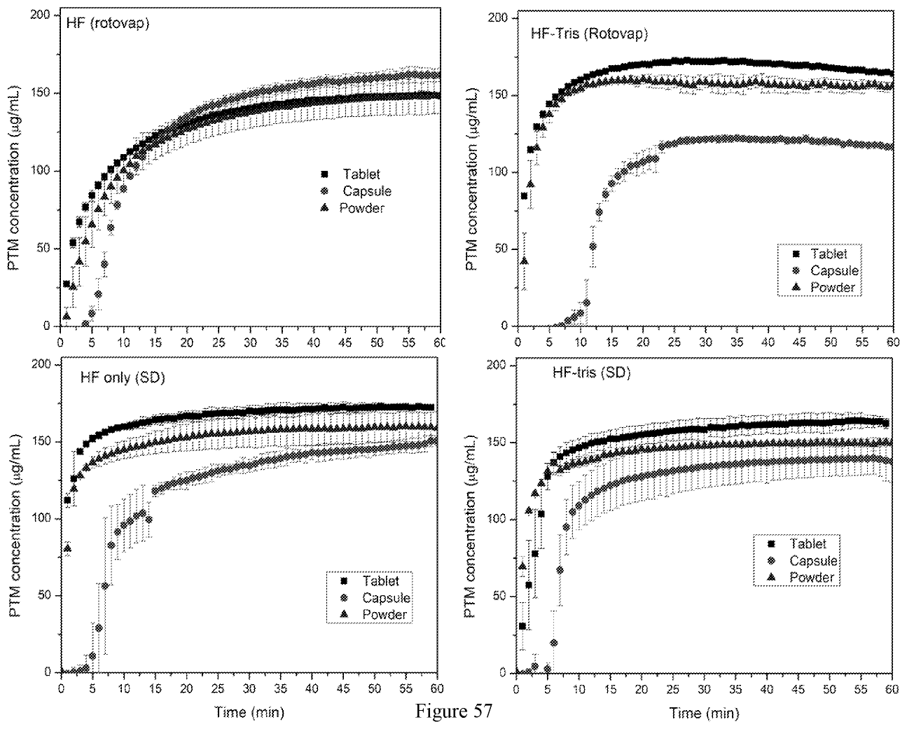

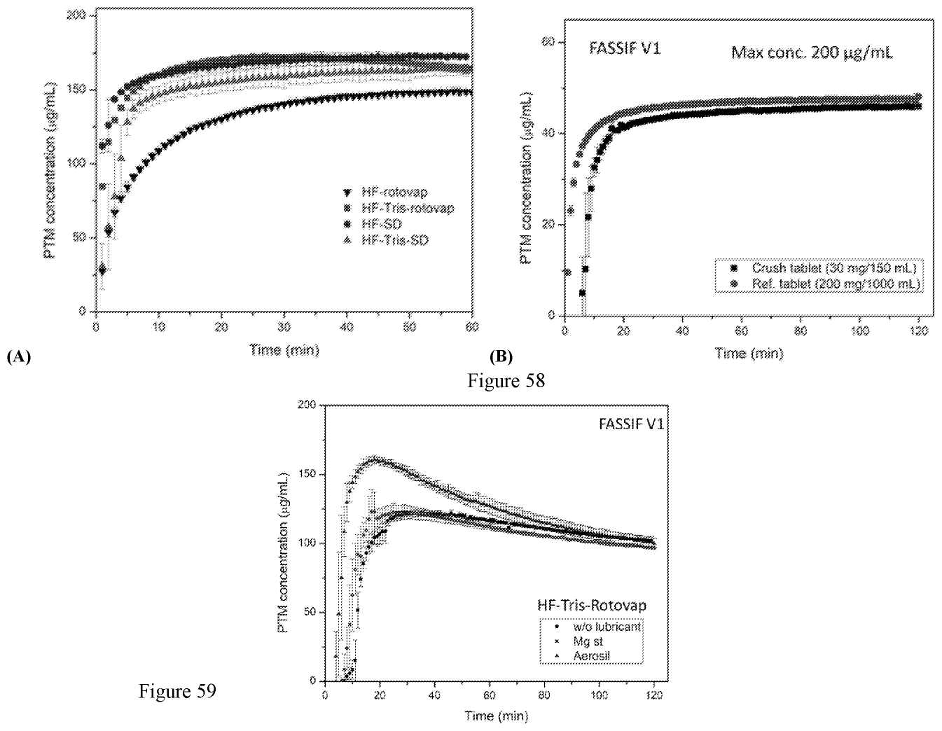

To study the effect of dosage form, dissolution profiles of ASDs prepared using rotovap vs spray drying in powder, tablet and capsule forms were compared. As seen in FIG. 57, there is a lag in release at the start with capsules. Samples of HF-rotovap could maintain the release profile comparable to tablet and powder forms, other ASDs showed a decreased release when they were added into capsules. In tablet form (FIG. 59), samples of HF-rotovap ASD released less drug (about 75%) at 1 hour compared to the other ASDs (about 80%), however, the release from all ASDs was significantly higher (about 75-80%) compared to the reference PA-824 200 mg tablets (about 25%). Crushed tablet (equivalent of 30 mg PA-824 filled in capsule size 0/150 mL medium) had a similar release profile as the whole tablet (200mg PA-824/1000mL medium).

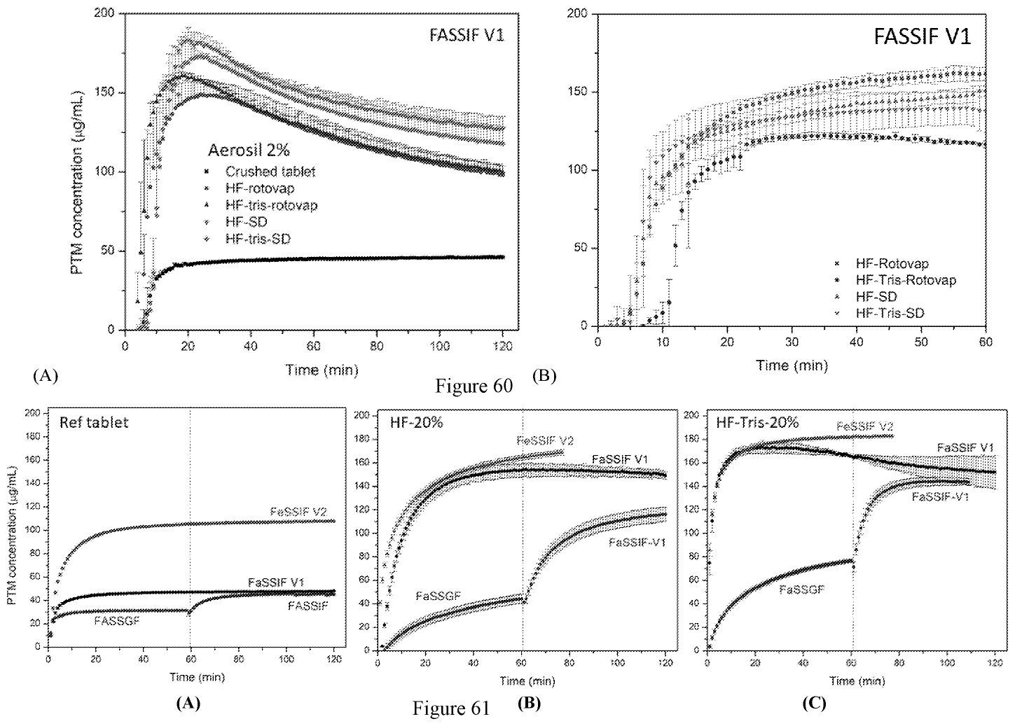

In order to improve the drug release of PA-824 from ASDs in capsules, lubricants were studied. Without being bound by theory, we hypothesized that colloidal silica (Aerosil) could attach on the ASD surface to protect the gelation and release the drug faster. Release profiles for these formulations are shown in FIGS. 59 and 60. Magnesium stearate did not have any impact on the drug release. Aerosil aided the disintegration of capsules by reducing gel formation which resulted in higher drug release, however, crystallization of PA-824 was observed after 30 minutes due to nucleation with Aerosil. In the absence of Aerosil, crystallization of PA-824 was not observed (FIG. 60).

In summary, ASDs exhibit much better release of drug in FaSSIF when compared to the reference tablet. However, the drug release showed a reduction for some systems if ASDs were added to capsules due to the gelation of the capsule contents. Tableting enables this gelation to be avoided and to maintain good release profiles from the ASD. The ASD of HF-Tris and ASD of HF-TPGS exhibited better release than the ASD of HF only, especially with samples prepared by the rotary evaporation method.

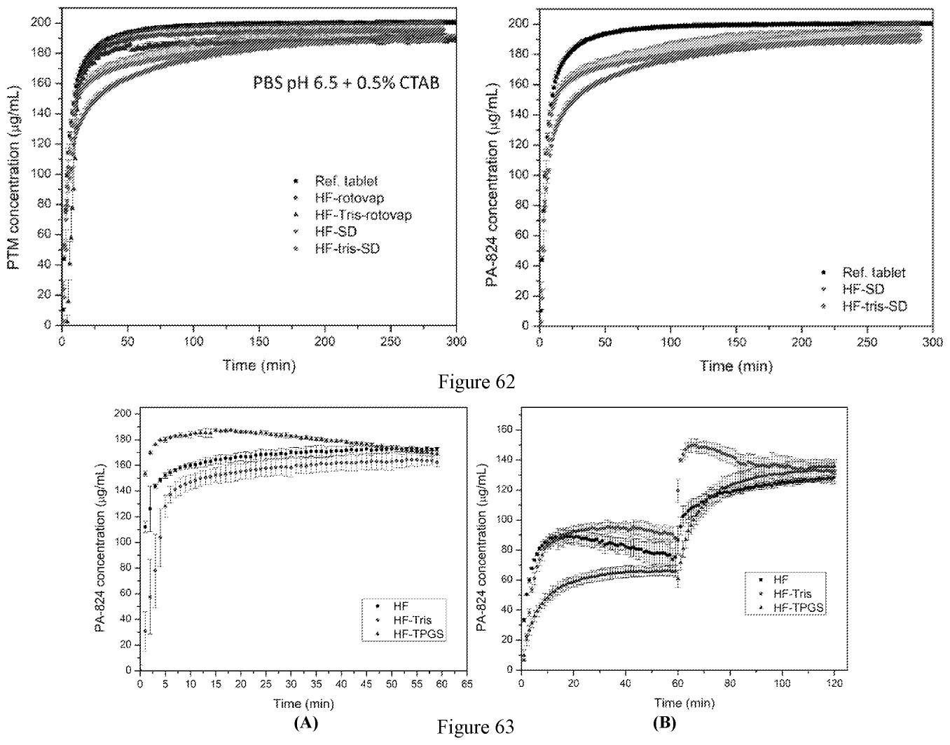

Spray dried ASDs share similar release profiles in FASSIF but a higher drug concentration was observed in pH-shift experiments with PA-824-HF-Tris ASD (FIGS. 61 and 63). In the presence of surfactant in the dissolution medium (0.5% w/v CTAB in PBS pH 6.5), there was near complete release (95%) in all cases, including the reference tablet, tablet of ASD with HF or HF-Tris (FIGS. 62).

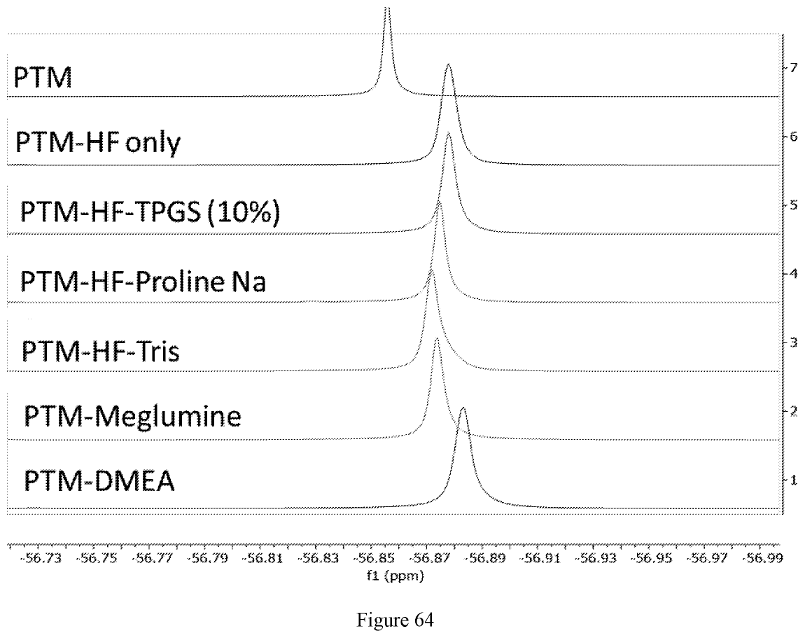

Stored spray dried ASD powders were tested for dissolution and profiles were consistent with previous results. No degradation was noted by NMR (FIG. 64).

The invention will be further described, without limitation, by the following numbered paragraphs:
1. An amorphous form of pretomanid.
2. The amorphous form of pretomanid according to paragraph 1, wherein the amorphous form is in a solid dispersion.
3. The amorphous form of pretomanid according to paragraph 1 or 2, wherein the amorphous form is in a nanospray dried solid dispersion or a spray dried solid dispersion.
4. An amorphous solid dispersion comprising pretomanid or a pharmaceutically acceptable salt thereof.
5. The amorphous solid dispersion according to paragraph 4, wherein the amorphous solid dispersion contains pretomanid, or a pharmaceutically acceptable salt thereof, in at least: 50% amorphous form, 60% amorphous form, 70% amorphous form, 80% amorphous form, 90% amorphous form, 95% amorphous form or 99% amorphous form.
6. The amorphous solid dispersion according to paragraph 4 or 5, wherein the amorphous solid dispersion is characterized by a DSC thermogram not having an endothermic event at 106° C.
7. The amorphous solid dispersion according to any one of paragraphs 4-6, wherein the amorphous solid dispersion is characterized by a DSC thermogram not having an endothermic event at 151° C.
8. The amorphous solid dispersion according to any one of paragraphs 4-7, having an X-ray powder diffraction pattern with peaks absent from, or of lower intensity and broad at, each of the 2-theta values of about 6, about 21, about 24, and about 30.
9. The amorphous solid dispersion according to any one of paragraphs 4-8, having an X-ray powder diffraction pattern with a characteristic halo.
10. The amorphous solid according to any one of paragraphs 4-9, further comprising one or more pharmaceutically acceptable excipients.
11. The amorphous solid dispersion according to paragraph 10, wherein the pharmaceutically acceptable excipient is a polymer.
12. The amorphous solid dispersion according to paragraph 10 or 11, wherein the pharmaceutically acceptable excipient is selected from one or more of: a polyvinyl caprolactam-polyvinyl acetate-polyethylene glycol graft copolymer (PCL-PVAc-PEG), a dv (HPMCAS), a vinylpyrrolidone-vinyl acetate copolymer, tocopherol polyethylene glycol 1000 succinate, Poloxamer 407, Sodium lauryl sulfate (SLS), polyvinylpyrrolidone (PVP), a high molecular polyethylene glycol, hydroxypropyl methylcellulose (HPMC), D-α-tocopheryl polyethylene glycol succinate (TPGS), sodium lauryl sulfate (SLS), a polymerized copolymer of dimethylaminoethyl methacrylate, butyl methacrylate, and methyl methacrylate or combinations thereof.
13. The amorphous solid dispersion according to any one of paragraphs 10-12, wherein the pharmaceutically acceptable excipient is a surfactant.
14. The amorphous solid dispersion according to paragraph 13, wherein the surfactant is present in an amount of 0.25%, 0.5%, 0.75%, 1%, 1.5%, 2%, 0.25-2%, 0.25-1%, 0.5%-1%, or 0.25-0.75% by weight.
15. The amorphous solid dispersion according to paragraph 13 or 14, wherein surfactant is cetyltrimethylammonium bromide (CTAB).
16. The amorphous solid dispersion according to any one of paragraphs 10-15, wherein the pharmaceutically acceptable excipient is a lubricant.
17. The amorphous solid dispersion according to paragraph 16, wherein the pharmaceutically acceptable excipient is a colloidal silica.
18. The amorphous solid dispersion according to any one of paragraphs 10-17, wherein the pharmaceutically acceptable excipient(s):
   a) are polyvinyl caprolactam-polyvinyl acetate-polyethylene glycol graft copolymer and poloxamer 407 and/or the amorphous solid dispersion is characterized by having an endotherm at 51° C., 100° C. and 137° C., as obtained with DSC,
   b) are polyvinyl caprolactam-polyvinyl acetate-polyethylene glycol graft copolymer and TPGS and/or the amorphous solid dispersion is characterized by having an endothermic melting peak at 106° C., as obtained with DSC,
   c) are polyvinyl caprolactam-polyvinyl acetate-polyethylene glycol graft copolymer and SLS and/or the amorphous solid dispersion is characterized by having an endotherm at 104° C. and 141° C. or an endothermic peak around 79° C. or an endothermic peak around 109° C., as obtained with DSC,
   d) is polyvinyl caprolactam-polyvinyl acetate-polyethylene glycol graft copolymer and/or the amorphous solid dispersion is characterized by having an endotherm around 105° C. and a wide endotherm at 141° C., as obtained with DSC,
   e) is hydroxypropyl methylcellulose acetate succinate (HPMCAS) and/or the amorphous solid dispersion is characterized by having an endotherm at 105° C. and 150° C., as obtained with DSC, or
   f) is vinylpyrrolidone-vinyl acetate copolymers and/or the amorphous solid dispersion is characterized by having an endotherm at 105° C. and a wide endotherm around 125° C., as obtained with DSC.
19. The amorphous solid dispersion according to any one of paragraphs 6-18, wherein the peak, endotherm, glass transition, endothermic event or endothermic peak is obtained in the first run.
20. The amorphous solid dispersion according to any one of paragraphs 10-19, wherein the pharmaceutically acceptable excipient is polyvinyl caprolactam-polyvinyl acetate-polyethylene glycol graft copolymer (PCL-PVAc-PEG) in combination with a second pharmaceutically acceptable excipient selected from a hypromellose acetate succinate (HPMCAS), a vinylpyrrolidone-vinyl acetate copolymer, Poloxamer 407, D-a-tocopheryl polyethylene glycol succinate (TPGS), and sodium lauryl sulfate (SLS).
21. The amorphous solid dispersion according to paragraph 20, wherein the weight ratio of pretomanid to the polyvinyl caprolactam-polyvinyl acetate-polyethylene glycol graft copolymer (PCL-PVAc-PEG) to the second pharmaceutically acceptable excipient is 30:60:10, 15:75:10, 30:35:35, 10:63:27, or from 20 to 30:from 50 to 80:from 5 to 30 (20-30:50-80:5-30), or from 10 to 30:from 35 to 75:from 10 to 35 (10-30:35-75:10-35).
22. The amorphous solid dispersion according to any one of paragraphs 10-21, wherein the weight ratio of pretomanid to the pharmaceutically acceptable excipient is 1:4, 3:7, 4:6, 1:9, 1:1 or from 1:9 to 1:1.
23. The amorphous solid dispersion according to any one of paragraphs 4-22, further comprising:
   a) a hypromellose acetate succinate (HPMCAS), optionally wherein the weight ratio of pretomanid to HPMCAS to MCC is 1:9:3,
   b) a vinylpyrrolidone-vinyl acetate copolymers, a polyvinyl caprolactam-polyvinyl acetate-polyethylene glycol graft copolymer (PCL-PVAc-PEG), microcrystalline cellulose (MCC), optionally wherein the weight ratio of pretomanid to vinylpyrrolidone-vinyl acetate copolymers to polyvinyl caprolactam-polyvinyl acetate-polyethylene glycol graft copolymer (PCL-PVAc-PEG) to MCC is 1:6:3:3, or
   c) a polyvinyl caprolactam-polyvinyl acetate-polyethylene glycol graft copolymer (PCL-PVAc-PEG), sodium lauryl sulfate (SLS), microcrystalline cellulose (MCC), optionally wherein the weight ratio of pretomanid to polyvinyl caprolactam-polyvinyl acetate-polyethylene glycol graft copolymer (PCL-PVAc-PEG) to SLS to MCC is 1:5:0.7:2.
24. The amorphous solid dispersion according to any one of paragraphs 10-23, wherein the pharmaceutically acceptable excipient is a hypromellose acetate succinate (HPMCAS).
25. The amorphous solid dispersion according to paragraph 24, wherein the pharmaceutically acceptable excipient is a HPMCAS-HF, HPMCAS MF or HPMCAS-LF.26.
26. The amorphous solid dispersion according to any one of paragraphs 4-25, wherein the concentration of pretomanid is 10%, 15%, 20%, 25% or 10-25% by weight.
27. The amorphous solid dispersion according to any one of paragraphs 4-26, further comprising a base, optionally selected from triethylamine, DMEA (dimethylaminoethanol), MDEA (N-methyldiethanolamine), triethanolamine, tris, meglumine or ammediol.
28. The amorphous solid dispersion according to any one of paragraphs 4-27 further comprising
   (a) HPMCAS-HF at 20% pretomanid by weight, (b) HPMCAS-HF and TPGS at 20% pretomanid by weight, (c) HPMCAS-HF and Tris at 20% pretomanid by weight, (d) HPMCAS-HF and TPGS at 25% pretomanid by weight.
29. The amorphous solid dispersion according to any one of paragraphs 4-28, in form of a tablet, a powder or in a capsule.
30. An amorphous form of pretomanid or an amorphous solid dispersion according to any one of paragraphs 4-29, wherein the amorphous form of pretomanid or the amorphous solid dispersion is formed by vacuum drying, nanospray drying, hot melt extrusion, nano suspension, nano suspension followed by lyophilization, or spray drying.
31. A pharmaceutical composition, comprising an amorphous form of pretomanid an amorphous solid dispersion according to any one of paragraphs 4-30, and a pharmaceutically acceptable excipient.
32. A method of treating a mycobacterial infection, comprising the step of administering a therapeutically effective amount of an amorphous form of pretomanid or an amorphous solid dispersion according to any one of paragraphs 4-30, to a patient in need thereof.

33. The method according to paragraph 32, wherein the mycobacterial infection is caused by Mycobacterium tuberculosis, Mycobacterium avium, Mycobacterium kansasii, Mycobacterium abscessus or Mycobacterium chelonae.
34. The method according to any one of paragraphs 32-33, wherein the patient is afflicted with tuberculosis (TB), multi-drug-resistant tuberculosis (MDR-TB), pre-extensively drug resistant (Pre-XDR-TB) or extensively drug-resistant tuberculosis (XDR-TB).

It is to be understood that the invention is not limited to the particular embodiments of the invention described above, as variations of the particular embodiments may be made and still fall within the scope of the appended claims.

What is claimed is:

1. An amorphous form of pretomanid.

2. The amorphous form of pretomanid according to claim 1, wherein the amorphous form is in a solid dispersion.

3. The amorphous form of pretomanid according to claim 1, wherein the amorphous form is in a nanospray dried solid dispersion or a spray dried solid dispersion.

4. An amorphous solid dispersion comprising pretomanid or a pharmaceutically acceptable salt thereof.

5. The amorphous solid dispersion according to claim 4, wherein the amorphous solid dispersion contains pretomanid, or a pharmaceutically acceptable salt thereof, in at least: 50% amorphous form, 60% amorphous form, 70% amorphous form, 80% amorphous form, 90% amorphous form, 95% amorphous form or 99% amorphous form.

6. The amorphous solid dispersion according to claim 4, wherein the amorphous solid dispersion is characterized by a DSC thermogram not having an endothermic event at 106° C.

7. The amorphous solid dispersion according to claim 4, wherein the amorphous solid dispersion is characterized by a DSC thermogram not having an endothermic event at 151° C.

8. The amorphous solid dispersion according to claim 4, having an X-ray powder diffraction pattern with peaks absent from, or of lower intensity and broad at, each of the 2-theta values of about 6, about 21, about 24, and about 30.

9. The amorphous solid dispersion according to claim 4, having an X-ray powder diffraction pattern with an amorphous halo.

10. The amorphous solid according to claim 4, further comprising one or more pharmaceutically acceptable excipients.

11. The amorphous solid dispersion according to claim 10, wherein the pharmaceutically acceptable excipient is a polymer.

12. The amorphous solid dispersion according to claim 10, wherein the pharmaceutically acceptable excipient is selected from one or more of: a polyvinyl caprolactam-polyvinyl acetate-polyethylene glycol graft copolymer (PCL-PVAc-PEG), a hypromellose acetate succinate (HPMCAS), a vinylpyrrolidone-vinyl acetate copolymer, tocopherol polyethylene glycol 1000 succinate, Poloxamer 407, Sodium lauryl sulfate (SLS), polyvinylpyrrolidone (PVP), a high molecular polyethylene glycol, hydroxypropyl methylcellulose (HPMC), D-α-tocopheryl polyethylene glycol succinate (TPGS), sodium lauryl sulfate (SLS), a polymerized copolymer of dimethylaminoethyl methacrylate, butyl methacrylate, and methyl methacrylate or combinations thereof.

13. The amorphous solid dispersion according to claim 10, wherein the pharmaceutically acceptable excipient is a surfactant.

14. The amorphous solid dispersion according to claim 13, wherein the surfactant is present in an amount of 0.25%, 0.5%, 0.75%, 1%, 1.5%, 2%, 0.25-2%, 0.25-1%, 0.5%-1%, or 0.25-0.75% by weight.

15. The amorphous solid dispersion according to claim 13, wherein surfactant is cetyltrimethylammonium bromide (CTAB).

16. The amorphous solid dispersion according to claim 10, wherein the pharmaceutically acceptable excipient is a lubricant.

17. The amorphous solid dispersion according to claim 16, wherein the pharmaceutically acceptable excipient is a colloidal silica.

18. The amorphous solid dispersion according to claim 10, wherein the pharmaceutically acceptable excipient(s):
a) are polyvinyl caprolactam-polyvinyl acetate-polyethylene glycol graft copolymer and poloxamer 407 and the amorphous solid dispersion is characterized by having an endotherm at 52° C., 100° C. and 137° C., as obtained with DSC,
b) are polyvinyl caprolactam-polyvinyl acetate-polyethylene glycol graft copolymer and TPGS and the amorphous solid dispersion is characterized by having an endothermic melting peak at 106° C., as obtained with DSC,
c) are polyvinyl caprolactam-polyvinyl acetate-polyethylene glycol graft copolymer and SLS and the amorphous solid dispersion is characterized by having an endotherm at 104° C. and 141° C., as obtained with DSC,
d) is polyvinyl caprolactam-polyvinyl acetate-polyethylene glycol graft copolymer and the amorphous solid dispersion is characterized by having an endotherm at 105° C.and a wide endotherm at 141ºC, as obtained with DSC,
e) is hydroxypropyl methylcellulose acetate succinate (HPMCAS) and the amorphous solid dispersion is characterized by having an endotherm at 105° C. and 150° C., as obtained with DSC, or
f) is vinylpyrrolidone-vinyl acetate copolymers and the amorphous solid dispersion is characterized by having an endotherm at 105° C. and a wide endotherm at 126° C., as obtained with DSC.

19. The amorphous solid dispersion according to claim 18, wherein the endotherm, glass transition or endothermic peak is obtained in the first run.

20. The amorphous solid dispersion according to claim 10, wherein the pharmaceutically acceptable excipient is polyvinyl caprolactam-polyvinyl acetate-polyethylene glycol graft copolymer (PCL-PVAc-PEG) in combination with a second pharmaceutically acceptable excipient selected from a hypromellose acetate succinate (HPMCAS), a vinylpyrrolidone-vinyl acetate copolymer, Poloxamer 407, D-α-tocopheryl polyethylene glycol succinate (TPGS), and sodium lauryl sulfate (SLS).

21. The amorphous solid dispersion according to claim 20, wherein the weight ratio of pretomanid to the polyvinyl caprolactam-polyvinyl acetate-polyethylene glycol graft copolymer (PCL-PVAc-PEG) to the second pharmaceutically acceptable excipient is 30:60:10, 15:75:10, 30:35:35, 10:63:27, or from 20 to 30:from 50 to 80:from 5 to 30 (20-30:50-80:5-30), or from 10 to 30:from 35 to 75:from 10 to 35 (10-30:35-75:10-35).

22. The amorphous solid dispersion according to claim 10, wherein the weight ratio of pretomanid to the pharmaceutically acceptable excipient is 1:4, 3:7, 4:6, 1:9, 1:1, or from 1:9 to 1:1

23. The amorphous solid dispersion according to claim 4, further comprising:
a) a hypromellose acetate succinate (HPMCAS), optionally wherein the weight ratio of pretomanid to HPMCAS to MCC is 1:9:3,
b) a vinylpyrrolidone-vinyl acetate copolymers, a polyvinyl caprolactam-polyvinyl acetate-polyethylene glycol graft copolymer (PCL-PVAc-PEG), microcrystalline cellulose (MCC), optionally wherein the weight ratio of pretomanid to vinylpyrrolidone-vinyl acetate copolymers to polyvinyl caprolactam-polyvinyl acetate-polyethylene glycol graft copolymer (PCL-PVAc-PEG) to MCC is 1:6:3:3, or
c) a polyvinyl caprolactam-polyvinyl acetate-polyethylene glycol graft copolymer (PCL-PVAc-PEG), sodium lauryl sulfate (SLS), microcrystalline cellulose (MCC), optionally wherein the weight ratio of pretomanid to polyvinyl caprolactam-polyvinyl acetate-polyethylene glycol graft copolymer (PCL-PVAc-PEG) to SLS to MCC is 1:5:0.7:2.

24. The amorphous solid dispersion according to claim 10, wherein the pharmaceutically acceptable excipient is a hypromellose acetate succinate (HPMCAS).

25. The amorphous solid dispersion according to claim 24, wherein the pharmaceutically acceptable excipient is a HPMCAS-HF, HPMCAS MF or HPMCAS-LF.26. The amorphous solid dispersion according to claim 4, wherein the concentration of pretomanid is 10%, 15%, 20%, 25% or 10-25% by weight.

27. The amorphous solid dispersion according to claim 4, further comprising a base, optionally selected from triethylamine, DMEA (dimethylaminoethanol), MDEA (N-methyldiethanolamine), triethanolamine, tris, meglumine or ammediol.

28. The amorphous solid dispersion according to claim 4 further comprising (a) HPMCAS-HF at 20% pretomanid by weight, (b) HPMCAS-HF and TPGS at 20% pretomanid by weight, (c) HPMCAS-HF and Tris at 20% pretomanid by weight, (d) HPMCAS-HF and TPGS at 25% pretomanid by weight.

29. The amorphous solid dispersion according to claim 10, in form of a tablet, a powder or in a capsule.

30. An amorphous form of pretomanid or an amorphous solid dispersion according to claim 4, wherein the amorphous form of pretomanid or the amorphous solid dispersion is formed by vacuum drying, nanospray drying, hot melt extrusion, nano suspension, nano suspension followed by lyophilization, or spray drying.

31. A pharmaceutical composition, comprising an amorphous form of pretomanid or an amorphous solid dispersion according to claim 4 and a pharmaceutically acceptable excipient.

32. A method of treating a mycobacterial infection, comprising the step of administering a therapeutically effective amount of an amorphous form of pretomanid or an amorphous solid dispersion according to claim 4, to a patient in need thereof.

33. The method according to claim 32, wherein the mycobacterial infection is caused by Mycobacterium tuberculosis, Mycobacterium avium, Mycobacterium kansasii, Mycobacterium abscessus or Mycobacterium chelonae.

34. The method according to claim 30, wherein the patient is afflicted with tuberculosis (TB), multi-drug-resistant tuberculosis (MDR-TB), pre-extensively drug resistant (Pre-XDR-TB) or extensively drug-resistant tuberculosis (XDR-TB).
